SEQUENCE LISTING (42 sequences)

SEQ ID NO: 1 - DNA, 1254 bp, Homo sapiens
atgagctctaagtacccgcggtctgtccggcgctgcctgcccctctgggccctaacactggaagcagctctcattctcctcttctatttttttacccactatgacgcttccttagaggatcaaaaggggctcgtggcatcctatcaagttggccaagatctgaccgtgatggcggccattggcttgggcttcctcacctcgagtttccggagacacagctggagcagtgtggccttcaacctcttcatgctggcgcttggtgtgcagtgggcaatcctgctggacggcttcctgagccagttcccttctgggaaggtggtcatcacactgttcagtattcggctggccaccatgagtgctttgtcggtgctgatctcagtggatgctgtcttggggaaggtcaacttggcgcagttggtggtgatggtgctggtggaggtgacagctttaggcaacctgaggatggtcatcagtaatatcttcaacacagactaccacatgaacatgatgcacatctacgtgttcgcagcctattttgggctgtctgtggcctggtgcctgccaaagcctctacccgagggaacggaggataaagatcagacagcaacgatacccagtttgtctgccatgctgggcgccctcttcttgtggatgttctggccaagtttcaactctgctctgctgagaagtccaatcgaaaggaagaatgccgtgttcaacacctactatgctgtagcagtcagcgtggtgacagccatctcagggtcatccttggctcacccccaagggaagatcagcaagacttatgtgcacagtgcggtgttggcaggaggcgtggctgtgggtacctcgtgtcacctgatcccttctccgtggcttgccatggtgctgggtcttgtggctgggctgatctccgtcgggggagccaagtacctgccggggtgttgtaaccgagtgctggggattccccacagctccatcatgggctacaacttcagcttgctgggtctgcttggagagatcatctacattgtgctgctggtgcttgataccgtcggagccggcaatggcatgattggcttccaggtcctcctcagcattggggaactcagcttggccatcgtgatagctctcacgtctggtctcctgacaggtttgctcctaaatcttaaaatatggaaagcacctcatgaggctaaatattttgatgaccaagttttctggaagtttcctcatttggctgttggattttaa

SEQ ID NO: 2 - DNA, 1254 bp, Homo sapiens
atgagctctaagtacccgcggtctgtccggcgctgcctgcccctctgggccctaacactggaagcagctctcattctcctcttctatttttttacccactatgacgcttccttagaggatcaaaaggggctcgtggcatcctatcaagtcggccaagatctgaccgtgatggcggcccttggcttgggcttcctcacctcaaatttccggagacacagctggagcagtgtggccttcaacctcttcatgctggcgcttggtgtgcagtgggcaatcctgctggacggcttcctgagccagttccctcctgggaaggtggtcatcacactgttcagtattcggctggccaccatgagtgctatgtcggtgctgatctcagcgggtgctgtcttggggaaggtcaacttggcgcagttggtggtgatggtgctggtggaggtgacagctttaggcaccctgaggatggtcatcagtaatatcttcaacacagactaccacatgaacctgaggcacttctacgtgttcgcagcctattttgggctgactgtggcctggtgcctgccaaagcctctacccaagggaacggaggataatgatcagagagcaacgatacccagtttgtctgccatgctgggcgccctcttcttgtggatgttctggccaagtgtcaactctgctctgctgagaagtccaatccaaaggaagaatgccatgttcaacacctactatgctctagcagtcagtgtggtgacagccatctcagggtcatccttggctcacccccaaaggaagatcagcatgacttatgtgcacagtgcggtgttggcaggaggcgtggctgtgggtacctcgtgtcacctgatcccttctccgtggcttgccatggtgctgggtcttgtggctgggctgatctccatcgggggagccaagtgcctgccggtgtgttgtaaccgagtgctggggattcaccacatctccgtcatgcactccatcttcagcttgctgggtctgcttggagagatcacctacattgtgctgctggtgcttcatactgtctggaacggcaatggcatgattggcttccaggtcctcctcagcattggggaactcagcttggccatcgtgatagctctcacgtctggtctcctgacaggtttgctcctaaatctcaaaatatggaaagcacctcatgtggctaaatattttgatgaccaagttttctggaagtttcctcatttggctgttggattttaa

SEQ ID NO: 3 - DNA, 17 bp, Artificial sequence
  Synthetic oligonucleotide primer
ggccaccaccatttgaa

SEQ ID NO: 4 - DNA, 20 bp, Artificial sequence
  Synthetic oligonucleotide primer
ccatgaacatgccacttcac

SEQ ID NO: 5 - DNA, 23 bp, Artificial sequence
  Synthetic oligonucleotide probe
ttttacagacgcctgctaccatg

SEQ ID NO: 6 - DNA, 23 bp, Artificial sequence
  Synthetic oligonucleotide probe
catggtagcaggcgtctgtaaaa

SEQ ID NO: 7 - DNA, 23 bp, Artificial sequence
  Synthetic oligonucleotide probe
ttttacagacgtctgctaccatg

SEQ ID NO: 8 - DNA, 23 bp, Artificial sequence
  Synthetic oligonucleotide probe
catggtagcagacgtctgtaaaa

SEQ ID NO: 9 - DNA, 18 bp, Artificial sequence
  Synthetic oligonucleotide primer
tcctggctctccctctct

SEQ ID NO: 10 - DNA, 18 bp, Artificial sequence
  Synthetic oligonucleotide primer
ttttcaaaaccccggaag

SEQ ID NO: 11 - DNA, 23 bp, Artificial sequence
  Synthetic oligonucleotide probe
ggtcaacttggcgcagttggtgg

SEQ ID NO: 12 - DNA, 21 bp, Artificial sequence
  Synthetic oligonucleotide probe
gtcaacttggcgcagttggtg

SEQ ID NO: 13 - DNA, 23 bp, Artificial sequence
  Synthetic oligonucleotide probe
ggtcaacttggtgcagttggtgg

SEQ ID NO: 14 - DNA, 21 bp, Artificial sequence
  Synthetic oligonucleotide probe
gtcaacttggtgcagttggtg

SEQ ID NO: 15 - DNA, 18 bp, Artificial sequence
  Synthetic oligonucleotide primer
tcctggctctccctctct

SEQ ID NO: 16 - DNA, 24 bp, Artificial sequence
  Synthetic oligonucleotide primer
gttgtctttatttttcaaaaccct

SEQ ID NO: 17 - DNA, 23 bp, Artificial sequence
  Synthetic oligonucleotide primer
gctctgaactttctccaaggact

SEQ ID NO: 18 - DNA, 20 bp, Artificial sequence
  Synthetic oligonucleotide primer
attctgctcagcccaagtag

SEQ ID NO: 19 - DNA, 24 bp, Artificial sequence
  Synthetic oligonucleotide primer
ttgaattaagcacttcacagagca

SEQ ID NO: 20 - DNA, 18 bp, Artificial sequence
  Synthetic oligonucleotide primer
caccttgctgatcttccc

SEQ ID NO: 21 - DNA, 20 bp, Artificial sequence
  Synthetic oligonucleotide primer
agtagtgagctggcccatca

SEQ ID NO: 22 - DNA, 20 bp, Artificial sequence
  Synthetic oligonucleotide primer
cttcagccaaagcagaggag

SEQ ID NO: 23 - DNA, 25 bp, Artificial sequence
  Synthetic oligonucleotide probe
ataaagatcagacagcaacgatacc

SEQ ID NO: 24 - DNA, 23 bp, Artificial sequence
  Synthetic oligonucleotide probe
taaagatcagacagcaacgatac

SEQ ID NO: 25 - DNA, 25 bp, Artificial sequence
  Synthetic oligonucleotide probe
ataaagatcagagagcaacgatacc

SEQ ID NO: 26 - DNA, 23 bp, Artificial sequence
  Synthetic oligonucleotide probe
taaagatcagagagcaacgatac

SEQ ID NO: 27 - DNA, 21 bp, Artificial sequence
  Synthetic oligonucleotide probe
ctggccaagtttcaactctgc

SEQ ID NO: 28 - DNA, 19 bp, Artificial sequence
  Synthetic oligonucleotide probe
tggccaagtttcaactctg

SEQ ID NO: 29 - DNA, 21 bp, Artificial sequence
  Synthetic oligonucleotide probe
ctggccaagtgtcaactctgc

SEQ ID NO: 30 - DNA, 19 bp, Artificial sequence
  Synthetic oligonucleotide probe
tggccaagtgtcaactctg

SEQ ID NO: 31 - DNA, 23 bp, Artificial sequence
  Synthetic oligonucleotide probe
gtgcacagtgcggtgttggcagg

SEQ ID NO: 32 - DNA, 21 bp, Artificial sequence
  Synthetic oligonucleotide probe
tgcacagtgcggtgttggcag

SEQ ID NO: 33 - DNA, 23 bp, Artificial sequence
  Synthetic oligonucleotide probe
gtgcacagtgcagtgttggcagg

SEQ ID NO: 34 - DNA, 21 bp, Artificial sequence
  Synthetic oligonucleotide probe
tgcacagtgcagtgttggcag

SEQ ID NO: 35 - DNA, 24 bp, Artificial sequence
  Synthetic oligonucleotide primer
acaaactccccgatgatgtgagtg

SEQ ID NO: 36 - DNA, 22 bp, Artificial sequence
  Synthetic oligonucleotide primer
gaggctgagaaaggttaagcca

SEQ ID NO: 37 - DNA, 23 bp, Artificial sequence
  Synthetic oligonucleotide probe
tgctggtgcttgataccgtcgga

SEQ ID NO: 38 - DNA, 21 bp, Artificial sequence
  Synthetic oligonucleotide probe
gctggtgcttgataccgtcgg

SEQ ID NO: 39 - DNA, 23 bp, Artificial sequence
  Synthetic oligonucleotide probe
tgctggtgcttcataccgtcgga

SEQ ID NO: 40 - DNA, 21 bp, Artificial sequence
  Synthetic oligonucleotide probe
gctggtgcttcataccgtcgg

SEQ ID NO: 41 - DNA, 64956 bp, Homo sapiens
gacaccccagccacgccaagccgggaagtccccgcctcctggagctgaacccgcccctctcccagaggtggagctgcggggggcgggaacaggcacggagaaaataaacaagactaaaaagtcctgagtagcgctgtgtggccgcaaacctgaacccaccttttgcaccacgcgggacccggcacgcttcctgccacccacccctgagagggctgcgcggccgaccccagtactagaaaacactcgtcacctcaatcaagacgggtacgaaggccaacggacgccttcctttagaacgctcagcacacagagcaacttctcacgcctactctcaaatggcgtactccaaactagcactcccgacgtccagctgtgaacccagagcggcggaaagcccctgaacccagcgcccgggcatgcgcagacgcgttgttgtggtgggcgtggctccctccggacccggcgccccgccctccgccccgtgtccgcatgcgcgactgagccgcgggggtggtactgctgcatccgggtgtctgaagatccgatgaaataacatatgcaaaatgattgggtccgtgattggcattccagaaatggtagctgttattcagccaacaaatatttattgagcacctactatggacttccctggtgctgaggatacaacagcaaccacagcagtcaaaagtccctgtcttcatgttgctcagattctcataggggaaagcaaataatgaacaaatacacggccgggcgcagtggctcacgcctgtaatcccagtactttgcgaggccaaggtgggcaagtcacctgaggtcaggagttcgagaccagactagccaacgtggtgaaaccctgtcactactaaaaatacaaaaattagcgcggtgtggtggctcatgcctgtagtcccagctacttgggaggctgaggaaggagaatcgcttgaacctaaaaggcagaagttgcaatgagccaagatcgtgccactgcattccagcctgggtgacagagtactccgtctaaaaaaaaaacctaaatacacaagtaaaaatatagacctcgtcagatgctagtaagtgctgtgaaggaaactaaaaggggaacacaaggaacccttgtcaaggggagaagaaaggggagttgatgctgtccttttaaatagggcagtcagaggccgggcacagtggttcacacctataatcccagcactttgggaggttgaggtgggtggatcacttgaggtcaggagttcaagaccagcctggccaacctggtgaaatcctgtctctactaaaaaaacaaaaactagccgggtgtggtatcacgcgcctataatcccagctactcgggaggctgaggcgggagaatcacttgaacctgggaggtggaggttgcagtgagccgagattgtgccattgcagtccagcctaggcaacaagagcaaaacttcatctcaaaaaaaaaaaaaaaaatagggcagtcagggaaaactttcctgagaaggggatggtggaggatccagggaggtgaggtggggagcaagccagtacagttgttccttgactttcgatggggttatgtcctgataaagccatggtaagtaggaaatattgtaagtcaaaaatgcatttaatacatctaacctacggaacatcatagcttagtctcacctaccttaaacatgcttagaacacttacattagcctacagttgggcaaaatcctctaacacaaagcctattttatgataaagtattgaatatctcatgtaatgtactgagtactgtacggaaagtgaaagacggagtggtgggatgggaactctaagcgcggcttccactgcatgtgtgttgctttcgcgccatcataaagttgaaaagcgttaagtcaaaccatcgtacgtcggaggccatctgtatctggtaggaggagtgtttcagacagagagaacagcaggtgcacagagtgcttttttcccagcattttattatgaaaaatttcaaacatctaccaaaaaaagttgaaagacttgtacagtgaaaagccatacatctcacagctagaatcaacaattaacattttactgtatttggtttttgacttatctatcctagatcccttgtgctttctgtagcaggtgacctgccttgaagatttaaagacagaatatcaggaaatgtagtcagaaaatggggccttttataagagtcagaggggaagagcaaaacctctgcttttgacaaatctgttgggagaggccaactgcagggatacctcccttttttaatgaaagcatttctgttctgcgaggagcgggatcctcttgtcaagcagtcagtccctgctgcttccttactggggcaggatcaggacgcacagggatttggagtgccttggaaccaaccaccacccacgccgtttgccagctggtaaacatgcccatcaggtccgggggttggcattgcctggacatctttagtgttcatcttgctgacatctggtgccctcgggcaggtaggtgcagttggctgcctggtttacagagcttgtactgggcccaggttagcagaggtcacatccatttatcccactgcgcagaggagttccttctcaggaaacccagtttataagaagtactgactgccagaaatagagcagaaatgagaaccaggaggcaattgtgagaggaatggagacttctgacctctggggattggggtaccctcccccttaattgctgttggggtagcagagggcttagaagcccatgttcctagacttttagaattggaagaagacttagaagtaatctaggctgggggtccccaacccccaggctgtggcccgttaggaacctgaccgcacagcatgagggataggccagcgagcactaccgcctgagctccgcctcctgtcagatcagcagcggcattagattctcataggggcacaaaccctattgggaaccgcgcatgagagggatctaggttgcgtgctccttaggagaatctaactaatgcctgatgatctgaggtggaacagtttcatccccaaaccatccctccaacctcaccccggtccatggaaaaattgtcttttacaaaacccgtccctggtgccaaaaagcttggggacccctgatctaggctacagttaagtggtcaaacacccaggtcctgaagttaggctgcctgggtttaaatcccagctctactgcttactagccctgtgaccttgagcaagtcacttagtttttctgtgcctcagttcactcatttgtaataaatcctaatagtacccatcccagtgtcatgaactaagttcatatatgtaaagtacttagaatggtgcctagcaagtacttaataacagttagctctgaaaatgtataaagcaaaattaaccaatgttttagtggtttgcagccaacttttttctatgcgtgtgctaacatattattttataagagtgggaatatattgtacatgctgttatataacttgctttttcactaaacagtctatcctctgtgtcagttttgataaaagcgttttcctcttgcttttcctgcatatgttcagaaccatcatattggtagcaagtttcatgtcctgtagttttcttaaccaaccccctgctagtggacatttaggttagtctcagttttttccttctgtaaataaagctgcactgagcaagaagtgactgatgccaagtgactagatgaccttaggtatgacctctctgggtcttggtttcttggtctaaaaacaaaatgacaggattcgactgggtgattaaaatctcctctgatctacataggaattgttttcaagacatttctgcattcctctagtgacagggtgctcactacctcatgagtatttcagtggacaactgtaatggtcaataaagtatccactttccacctccctgcagctcctggccctggctttattctctggggctccacacattcagtttacactcagtggccagtggctgggaccattgtagaaaataaggaaactccaattccttccttcttttcttcctctttcatctcttcctccctctctacatccctctctctcttccttccttcctcgacacttaccatgtaccagaccttctgccaggcacatggatgggagcacaggggaagttggctgcagggttagaactaagtcccaagccccctaaagctcatgccaggggactggactgtccagtactgagggatggggatgctgaggctggtggccttcctcaaatgcactgtagtgccccaggcagagtcctgggctgccctgtgaggaggtgaccagaggtagagcaacttcaccctaaggctggatcaggatcccctccaggtttttactagagccaaacccacatctcctttctcttctgccacccccccttaaaatgcttagaaacacatagatttaaatacaaattcaaatgtaagtaatttcaactgtgtaactatgaggagtcagttctacgtgggtcctatctgtatcctccccagggctcagctccattctttgctttcattcattctcattcaatacattgttgttaagagctcactgggtgccctctctgtcatgtagtaaggttttaaaaagaaagcctcttctgagcttcagtttccttattcataaaataggagtattgatccattccttgcttttcttacaaggatatgctgaagatgactgaagtacagagtaaagaaggattatgtttgggtgtcaaaggaatagaatgccctctttcaaactgagcacagcaggaacctgtaacaggaacacagcaacttgttgaatgaatgacaatattggaaaacatacatttcctcccctccccatcatagtccctctgcttccgtgttaactccatagagaggccagcacaaccagccttgcagcctgagataaggcctttggcgggtgtctcccctatcgctccctcaagccctcaagtaggtgttggagagaggggtgatgcctggtgctggtggaacccctgcacagagacggacacaggatgagctctaagtacccgcggtctgtccggcgctgcctgcccctctgggccctaacactggaagcagctctcattctcctcttctatttttttacccactatgacgcttccttagaggatcaaaaggggctcgtggcatcctatcaaggtgagagttcattggaaaagtggtcacaggagcaaatagcaggggcaggggcgggggaggcctgtggttctccaggggcacagatgttcctttctacaaaatcccaaggaaaaagattcccccatcttcttccgtagattgcaccgaaattcagccaacaatgtaagctttcctttagaagcagcctgggcatgccctcttctgtgaagcctgccttgatttttcagcacagtgagaggcatcctctttggtgttcctcaaattccctctaccaaatggtcttcataattctctgcttctctgcttccccttctctctcctcagtggcaaggaatttttttatttttatagatttaggggatacaagtgcagctatcttatgcaagcaatttcatgttgttgggtttttggtttttgtttcctttttgtggcctctcgctcatttcttatttctttttgaggcagggtctcactctgttgcccaggctgaagtgcagtggcatgatcatggttcactgcagccttgacctcctagtctcaagcaatcttcccacctcagcctcccaagaagctgggaccacaggagggcaccaccatgcctggctaattttttttttttttttttttggtagagatgtgggtctccctgtgtttcccagactggtctcaaactcctggacacaagcgatcctccagcctcagtctcccaaagtgctggaattacaggcgtgaagcactgtgcccagctctcttgctcatatctatactagttttcttttggaagcttcagcctgttgctaccccccacccccacccccaccgaccccagctttcttctcacttaggggctgggaagtctgcatgctgtctataaatccagaaccagaaggtatggctgaaggggagggtaggatgatggttattttatattcagctaaaaatattcccagactgtgatgagacaactgtaaataagacagatgtccacaatggtgtgactttgcttttttaaaaatattgaaatgagtttcaggcatctcagtgggctgataggttgttgataatagacagggcctccttgaagaatgtccctgagacaaagttgaagcttgagcctggttgagtccttgcttgttcctaggttgatatgaacggctagttaactggaagcaaagagaagtcatcctgggggccatggcagtgacaagtaggacttagggagggaagcccttataccatttaaggtgctggcccagagaggagccttcagtgacagacaaacaagagctggcacaattttaattcacttcaatttactctaattcatttcaatccaatacaattcaatgcattccattcattcaaccatgtatgacatccaatgtgggatccagactcatgatgattagagctgatatttatgagcacttactatgtaccaggcactattctacatgctttacattgaaccctcacaataacccaatgaggtgggtactattatgatcttcgtttttcatatgaggaaactaggcatatggatgttgagtaatttgcccacggtcgctcagctagcaatagcacagcgtatttaaatttagccaccctggatttagtttccttacacttaaccattatgcatcatggccccattttacagtgggcttgagtctttgtcatataacccagtaggttagcagccactattccaaccctgtagattgactctagggtccatgttctttacccctgcaccgtgctactaacgtaggtacaaaatgtcctcagaaactcactttatacggaagctcagaggagggtccacaacccaggcaggggagacgatggtgtcaggggagggaggtgactgcccagccaggtcttgaaggctcagtaggaattacctgtgggacaaaggagggtcatccaagtgagggcacagtgggtgccatggcgtgcacacacaatagagcagactgagcctgggcttaacattgcattgccctggagcctaaaaggggaaacaaagggccgggcgacgtggctcacgcctgtaatcccggcacattgggaggccaaggctggagaatcacctgaggttaggagttcgagaccagcctggccaacatggcaaaaccgcatctctactaaaattataaaaactggctgggtgtggtggcacacgtctataatccgagctacttgggaggccattacactccagcctgggcgccagagtgagacttcatctcaaaaaaccaaacaacaaaaacaacaacaagaacaacaaaaaaacaaagaggagagcagggactgggtgtggtgactcatgcctgtaatcccaaacactttgggagaccaaggcaggcagatcacctgaggtcaggagttcgagaccagcctggccaacatggtaaaaccctgtctctactaaaaatacaaaaattagccggatgtggtggcacgtgcctgtagtcccagctgcttgggaagctgagggaggagaattgcttgaacccaggaggcagaggttgctgagctgagaacatgccactgcactccaccctgggtgacagagtgggactctgtctgaaaaaaataatagtaataaataaaaataaagagggaagcagcgggtggcagactcactgggctgcatacgaagtttggcttcagtctgaggtccgaatagtaaacagcagcgagacaagtttgggtttgggtcatggaggaagccatgccagggctggtgttgggcacagggaaaggggcatggcttgagacaccagaccagcgtggaggctgtagtgtagtattgacctgaggacttcaacattctgatggtgtacacacgattttttgagcatgtaccatggttatatattacactttaagtattactttaagtattactacattaatatattttgtatgttacaataaatacatacaaattaggaaaattgaaagagatcaaaatgaaatatataatattttcaaattactaatcataatggtgtcaatctccaggcagggtccattgctacagttgacgatagtggatgaaaattcactcctcagagtcttcttgataatttgaaattgtcttgattgacttgtcagatctgattagatcaacatgttttaaatctcgaatgtgactgacagcttgtacgaggagaagtttcactctgccttttcccttttgttcacttgactgccattatttctatgcttccaatctgtgtttttctgcacgagttggttaagccattacttcattttgtgaaagtttgttgagttaaacttaggtaacttaatctgtcaatccacttaattgaattcagtcctggtaaactataatagattattcaaacctgccaattctaaaaagacattttgagacaatcaggaaatctgaatatagcatgaatatcttacgatatacaaggattattgttaattttgttaggtatgataaaagcatggtgggttgtttttgtttttgttttttaagtctccatctgttagagaggcacattgaaatggcatgatatctggggtttgcttttatgccagaaaaaagaaaaagtacagaaggattatagaaacaagattggtctcatgtgacaatcatcagagtttggagatgggcacgtagggtcatcgtgctgttctctctgttttcgtatatgctttaaaagttctgtaatagttaattaaaaaaaaaaaaaaacaccctggctgagcatttagggaggccaagtggggaggatcgcttaaaccaaggagttcaagacgagcctaggaaacatagggagacccccccccatctctaaaaaaaaaaaaaaaaaaaaaaactttaaaatttaacccagtgtggtggcacatgcctatagtcccagctactcagtaggctgaggtgagaggcttgcttgagcctgggagcttgaggctgcagtgggacgggattgtaccacttcactccagcatgggcgacagagcaagaccctgtctcaaaaaaaataaaaatatttgaggtgaagcgaggctgtaataacaaatttaaaaatataaataaaacataaaggctgggtgtagtggctcacgcctgtaatcccagcactttgggaggccaaagcaggcagatcacgaggtctggagatggagaccatcctggctaacacgatgaaaccccatctctaccaaaaatacaaaaaaattagccgggtgtggtggcgggtgcctgtagtcccagctacttgggaggctgaggcaggagaatggcgtgaacccaggaggcggagctttcagtgagctgagattacgccactgcactccagcctgggcaacagagcgagactccgtctaaaaaaaaatgaaaataaaaataaatgaaacataaaaccctgccattagttgcaatatgaagaatatagagaaatgcatatcaaatccttctcattggaccaatattcccttagggcaccttccaaagctaggagactcaaggctgtatgacatcctgagcaagtgaggggtggcttctgggtgaatctgaatattaaatatttgcagaattgaaaacttcacaaagtacctttagagatagaatagcctagatccatgtttctcaaagtgtggtccccagacctgctgcctcagcatctcctggaaatttagtagaaatgcagattctcaggccctaggccagacctactgatcagaagctctgggcctggggcccagcagtctgtgttttcacaagccctcttggtgattcttctgtgcatgaaagttcgagaattcctggagctagactgattcaaatcttgcctctgtatcttagagaccttgggcagattagtcaacctctttctgcctctgtttctacttctgtcagaggatgatagtacttgtttcattaagttgttgaaaggataaatgaattgacacacataaagagtattagcttttattatcaaaagctttttttttgagacagagttttgctcttattgcccaggggagtgcagtggtgcgatcttggctcaccgcaacctccacctcccaggttcaagtaattctcctgcctcagcctcccgagtagctgggattacaggcatgcgccaccacgcccggctaattttgtatttttagtagagatggggtttctccatgttggtgaggctggtctcgaactcccaacctcaggtgatgcacccgccttggcctcccaaagtgctgggattacaggcgtgagccaccgcgcctggcccaaaagctttaatttcttaattttttaaataaaataaataaaactagaattgcttgttttcttccagctaccctggtgattgtattgagcattttctggggtgtgtgttctttgctgtaatgactactggtctggatgacctgtgatgagaccagatgggcaggggcagtggaggagattctagagatatttaggagataagtcagctgtacttgatgaaaagagtggggagttaaggctggctgcagatgtatgatttggcatagagaggtgccagttcctgagatgagagacagaaggggagggacaggttgtgaggatgaatgaacaatgatatgttcattctgggcttggagttaaggggcctatgatatgcttaggggaagcagagagtatcaattacctattgctgcataacagccaccccaaacttagtggcttaaaatagtaaccttttaatttactcatgatcatgattctgtggtgcaacaactgggctgggttcagctgggcagttcttctgttagtttcacccagggtcattcatgcatctgcagtttggggtgggatggcctcagatgacctcattcacgtgtttggcagttggtgattcactgggggccattactgtaacaatcgcctaccaggcagagcttccctaaggcttccaaactaggagactatcctgggtcctgtgctgtggataccactcagtcccccatccccaccccatattcctcaaaggcagagagaggggctactagaagacagaggagttttcccagtgacatgtaaacactccaaaccctggcaccttccacactgcagctttggtctgcccctttgggaaatctctgtttttcttcccaggctgctggaggggtgagagtcgccggtagagtagaggctgtgggcgaggaggtggcggcctcctgaggctgcagtggtctttccaggcagcagtgggagcacagggtggaggtcaaccctagagcctgggagagtgaagctgggtgtgacttcagagctgttggtgctgaagtttctgcaggccagaaggaggggcaagagtgggagggggcgcagatccagaatcacggaggcagctgaccggaggaggcagctgcccaaggggatggactcagaaggccaaagtgctgttatccaaacgaactctttgcaagtggtctctttgcaacaggcctgggggagagcagtcttgcctaaagtcacaccgctaatcagcggccggcacggggtaacagttactaacactcactacgtacccaatgctgggcgaagtgacttgcatgagccagcgagctcaatgctcatggcaatcctctgagcagctggcattgtttcatctcaattttacagctcaggaagctgggacacagaggaagagccaggctctgaacactgacaacctgattgagagacccacactgttcatcaccgttacgctatatatgctgtatagaaaggcaggatggcataatggttaaacctaggtaggtagggtttgaatcctcctgctaccatttactagctctgtgacttggactagttatagcacctctctgtgcctccctttccccatctctaaaatggggataataaatcgtacctcctacctgaggctgttgtgggctaagtctgtaaggcacgtagaacagtgcctggaacgtggggtactgtctatctgtgtgcctgctgttacaacaatggtgagtattgccttatctctcgctgctgaactaccaggttagacttctttctgcaagtcatgaggctttcataaacttttcctgaaggctttccgtagaatgtacaattcccctctgggtccaggcatgggcgcccgggtagcacatccacttcttatcacccctgaacaccttagagcccatcagcttatcaaaccagcagctgatgtgagtgcagagcagactgtgagaggtggaggctgataccagtgaggatgctccaagctgggacccagccctgaagcgggagcccagataatggatgggtggaaatgggcctggagcccaggagaagtgggaggatgagggggcagggggaggagaagcctgaaatcaaatgttatttcctgaccagtttggggtgcatgagctctgtcaacagctcatggaaactgctgccctaatttcatcttgttggctgaggcacaattcctctctcagggacagtgtagagccttggggaggaaggccctgagcgcgtatacctggaatcagggaatcgggatcaggggcagcagctgtgcccaataaagcccccacccaggatcctctgacttcctcatctctttttttttttttttgagctgcagtctcactctgtcatccaggctggagtacagtggtgcgatctcggctcactgcaacctcagccttctgggttcaagcgattctcctgcctcagcctcctgagtagctgggattacaggcatgcgccaccatgccaggctaatttttgtatttttagtagagacggggtttcaccatgttggccaggctggtctcaaactcctgacttcaagtgatctgcccacctcagcctcccaaagtgctaggattacagacataagccactgtgcctggccttttttttttttttttttttgtaaacagggtctccctctgtcacccaggctgctggagtgtagtggtgtgaccgcagctcactgcagccttaaccttctaggcacaagccatcctcctacctcaccctcctgagtagctgggactacaggcactcgccaccacgcccaagtaattttgtattttttgtagagacaaggtcttgctatgttgcctaggctggtcttgaactcctcagctcaagcaatcctccctccttggcctcccaaagtgctgggattgtgctgggattacaggtgtgagccaccatacctggtctgacttcctaatctttagggccccaactctgcccttatccaggcaactctcctctccccatcttccactaacttctttggaatattccagagctgtaaaagccttagagagtatcaagtccaactcctatgtgttacagacagggaaactgaggcctaaagagggtaatggacttgcctaagatcacttagtgaggtgagagaagaaagagctagagacagcctagcctgtgcaaggacatagttccaggcattcagagctgggctctgctgccggcatgtttggggcctggtagttagttcactgctgaactaccaggttagattttctttctccaagttgtggagctttcataaacttttcctgaaggtcttccttacaatgtacaattctcctctgggcccggtcatgagcgcccctcacaggctctctctggtccccttctgtaaaatgagaggaaaatggaagaattgctctactcatggaatcttcaataagtctgggccctatgcatatagcattgctacaaaatggcagatgcactttaacaatcgtgtttaataaaaggttggatttgcatatctgaagtggggcatgcagtctccaactgaacacaagcctcactgctcccgcatgtgcactgcaccttcatatacatatttcctgcttggctcctgagggaatttgagtaatcccaagaggaacccctgtagaaaatgtcccctggccacacacccccattcctaaggatgcaagcaggagatagaaacattccctgcacctccctccttgttgtcagaagaagtgcaaagagttgaatccttcctaatgcccacttctcacccacgccccaaatccccaggtcccatggaggtccttgggggcctcctatatcctggtggtgtcaggttgatttggaaatgtcagtgtcctcccttgtcctctctggcagaccctgggtatgtgtatgtttcaatggaagtgaatttaaatgtactttataaatcaaagactttttctgagactttggagagttccagtaatgagagcttctcattgttatcaaggccagggctggagaccagtggcaggtgagttcctattgctgtgattgtcatgatgatgttgatgaacagtcactatttattgagcgttctccatgtgccagtcactgtactaaacattatttcctttggatttcccagaaacctctcaggtgggtctaattacccttattcagctgataaggaaagtaagcaacttacaagaccacagggctatgaagtggaaacacataaattgatatttcattttatttatttatttattttgagacagagtctcactgtgtcgcccaggctggagtgcagtggtgcggtctcagctcactgcaacctctgcctcccgggttcaagcgattctcctgcctgcctcccgagtagctgggattacaggtgcccaccaccacatccagctaatttttttgtaattttagtagagacggggtttcaccatgttggccaggctagtctcgaactgctgacttcatgatctgcccacctcatcctcctaaattggtatctttatatgtccaaaagagtcaactggtggcaatttagtgaggtttaatctaataggaaatgatagagctgggatcgaacagagccatgtgaactcaaaacctatgcttccccttccacctttttgaaaaacattgtctaggctgggcacgatggctcatgcctgtaatcccagcactttgggagacggaggtgggtggattacatgaggtcaggagttcgagaccagcttggccaaaaattagccaggcgtggtggcgcgcgcctgtggttcccactgaagcacaggaggctgaagcacaagaatcacttgaacccgggaggtggaggttgcagcgagccgagatcgcaccactgcactccaacctgggcaacagagagactctgtctcgaaaaaaaaaaattgtctacatgctggttgcagaaaatttaaacactaaaactaaaaaagtaaaacacctcccaaacttagagacaatattaatgacggaaaaaaaattcttcaagatctctctctctccagtcatttattcatgtgcgaaaacagttggtgattattgataaaatagcttttagagtttggagcaattatgtgcattacatataccatttgattctggcaacctaatgaaggagtatgatcatttcccctatttaacagacaagaacaagaagagggagggcagatggtgtggtagtctaaggcacaggctccagcagattatctaggtgtaaatcttggctgtaggccaggccctgtggctcatgtctgtaatcccatcactttgggaaaccgaggtgggcagatcacttgaggtcaggagttcgagaccagcttggccaacatagcgaaaccccttctctattaaaaatacaaaaattagccgggcacggtggcaggcacctgtaatcccagctacttgggaggctgaggcaggagaatcacttgaacccaggaggcagaggttgcagtgagccaagatcttgccactgtactccagcctgggtgacgagtgaaactctatctcgatattaaaaaaaaaaatcttagctctacccaccggggcaagttacgtaacgcctctgtgccttggttttcatatctgtaaaatggtgacagtaacagcacccacgtcaaagtgtggttgtgagaacgaaacaagatagtctatgtaaagtgattaaaacagcgtaggcacatggtaaacgcttaggaaatgtaggctgttataaagctcagagatgttaagtaactagatcaagatcacacagttagagggtgccagagtcctgatttgaacccaagtttgtctcgttctggagctcaagctgctaaccctttttcaaaactggaattaaaccaaagtgctcaccctccgctttgctgggcccctccctgccctcaggtgcgtctcttccactcacctgccacagcagcctctgctcagggtctgagaccgggaaaggtgagggctacccaggtggccctgatgttttctgccagccagctcaccaggtccctcgcagcaggcggcaaagggagggaggtttgctgtgaagattatgtggttcccaacaacaagagcgctgggcctatctctgccctctcttttctgtgtgtcctgggacaagtcacttggcttctgtggcttcattttctcatgtgcccagccagggggttggccctcatatgcaataacagcagcaatgacctttactgagtgtccatgtgcgtcaagcacgtgtgctttacacttgttcttattattaggtttaataatagaataattgccacatttactgagcactcattatgggccaggccctgccctaagtgcttaattagctttagctcctctaatccttatcttatccccacacggcatgttatgttatccccattattcagttgagaacattgaggctcaaagaggcaaagtaacttgaccaaatacttgtaaacgatcttgcatgccccttccagctgccatttagtaagactctaatttcataccaccctaaatctcgtctgcttccccctcgtccttctcgccatctccccaccgagcagttggccaagatctgaccgtgatggcggccattggcttgggcttcctcacctcgagtttccggagacacagctggagcagtgtggccttcaacctcttcatgctggcgcttggtgtgcagtgggcaatcctgctggacggcttcctgagccagttcccttctgggaaggtggtcatcacactgttcaggtattgggatggtggctggatcacttctgggtcatagagggaatggaccccgaaaggacaggttccagaagatctgggatattgccccctctctgtctagcaccagtgctgtgcaatatttaggacatccttatactaaaagattattcattgtttaaaattcaaattaactgggcatcctgtattttactggacagccctactccgtgtatcacaaggaatccaggcctacattcctcctgcatcctttctttcctgttattgtcgattatgattttgtaaagttacataatcaatataagtttatggaaaacgtaagaaggaaacacgttagacagagagaaatagacatgccacacctagagagacattctatttttttttttttttttgagacggagtttcacttttgttgcccaggctggagtgcaatggcgctatctcggcacaccacaacctcagccttctgggttcaagcgattctcctgcctcagccgcctgagtagctgggattacaggcatgtgccaccgcgcctggctgattttgtatttttagtagagatagggtttctccgtgttggtcaggctagtctcaaactcctgacctcaggtgatccgcccgcctcggcctcccaaagtgctgggattacagacatgagccaccgcgtccagcctgagagacattctcttgaaaagaaaggactttcagccccctaatgctgctagacaataaatagccatgcctttattttcattaaattacctgtgctttgtttacatgcatttgtgtgaaatgctaagaaccatcacaactaatgtatggtgccagaagtcagaatagttgttacctgggcaggaggtggatattgattaggaaggaacacaaaataaccgcatggggtgcagaaaatgttctctatgttcacctgggtgatgattacacatcaagctatacacgttttaaaagggcattggcacttaataggaggaagtaggctaaattttttcctgaaacattgttttgttttgttcaaacctctgaatccctgtgctgcccagatgatggtaaacgtcatcctaggcatcttagggacctctcaaggccattccagcctccccttctaagaccctgctaaacctctgggcactgctgttaaacatttctctatgagccaggaactgtgctgagcactccacaaatattattttgtttaactcttccgggtagggatctaacctggtatacaggtaaggaagtggaagctcagagagggcaaggcacttgcctagggccacacagctaagtggtggagatggctccaactttttattataaccttttccacatgctccagagtgctcagaacatgaaacacagtctagccagctcccgattggccctggagggaaaaaactttatatatttttcttttttaaaaggtttagaggctgggcatggtggttcacacctgtaatcccagtacttttgggaaccgaggtgggcagatcacttgagcccagaagtttaagaccagcctgactaacacagtgagatcctgtctctgcagaaaatagaaaaatcagctaggcgtggtggtgtgcacccacagtcccagctacttgggaggctgaggcaggaggatcacctgaacccagtgaggttgaggctgagtgagccatgatcgtgccacttcactccagcctggacaacagagtgagaccctgtctcaaaaaacagttttaggggccgggcgcagtggttcatgcctgtaatcccagcactttgggaggccaaggcggggggatcatgaggtcaggagatcgagaccatcctggctaactcggagaaaccctgtctctactaaaaatacaaaaaattagccgggcgtggtggtgggcgcctgtagtcccagccactcgggaggctgaggcaggagaatggcgtgaacccgggaggcggagtttgcagtgaaccgagatggtgccactgcactccagcctgggtgacagagcgagactccgtctcaaaaaaaaaaaacaaaaacagttttaggccaggcgcggtggttcatgcctgtaatcctagtactttaggaggcctagcaggtggattacctgaggtcaggagtccgagaccaacctgagcaacatggtgaaatcctgtctctactaaaaacacaaaaattagctgggtgtggcggcaggcacctgtaatcccagctacttgggaggctgaggcaggcgaatcacttgaacccgggaggcggaggctatagtgagccgagatcgcaccattgcactgtagcctgggcgacagagtgaggctctgtctcaaaaacaaaacaaaacaaaaacagtctatgagttaattcccaccagaattcaatacacacacgcacacatgcacgcatacacacactgtgtccacctgggaagtgacaaagggcaccctgggggatttcaaatggtggtggccctggtttggtgttgctgccttagcttaaggtcacaccagccttcagcctcctgccccacagtctagggctgctcccctcatctgatgtccacagggacctgtttgttcttgactcaatctagaaagacgagaagggagagaagtcactcgcagcctgagtgaactcccctgccccacccctgactgcttggatccccctaggggtgacccctgctgaaactggctccttcctgaccggttcccgtcagggctgtgctgatgggtggtgcccaggcctgcccctggggacggggtactctcccttggcaacactccagcttgtgccacttgacttgggactgatttggttctgttttgagtcccttcaggggaggggcctatcttattcaacgttgttgtttgttttcctcacatactgataacttagcaaatggctattggagcaaaaatgaaaataaacggaactctgaagtgggatgttttaaaattttatttatttttttagagacagggtcttgctctgttgcccagtctggagtgcagtggtacaatcatagctcattgcagcctgtgcctcctgggctcaagtgatcctcccacctcagcctcctgagttaaatttttttacaggcgcctgctaccatgccctgctaatttttgtatttttagtagacaaggggtttcaccaggtgggtcaggttggtctggaactcccgacctcaagtgatccacctgcctaggcctcccaaagtactgggattacaggcgtgagccactgtgtccagcctaaaactgtttttgagacagggtctcactctgttgtccaggctggagtgaagtggcatgttcatggctcactcagcctcaacctcactgggttcaggtgatcctcctgcctcagcctcccaagtagctgggactgtgggtgcacaccaccacgcctagctgatttttctattttctgcagagacaggacctcactgtgttgctcaggctggtctcaaactcctgggctcaagtgatctgcccacctcggctctgaaaagtactggaattacagcctcctgagtagctgagaccacaggcacacaccaccacacctagctttttttttttttgctttttgtagagatggagtctcactatgttgcccaggctggtctcaaactccaggccttaagcaatcctcccacctcagcctcccaaagtgcgaagattacaggtgtgagccaccattcctggccttaaaagtgtgatatttttaatgtattttgaaatctgcaggactctccctagaagataatagcaataaccaactcctttattgtgcttgacgtatatcaactcactttgcccttaccgtggctccagaggcattgggtccaccttataaatggaggcaccaaggcacagagtgattaaataaattgcccaggatcacacagccagaaagtgtctgagtcaagattccagcccaggcagcctagacctgagagcacgctcctaaccactgcacatcactgtcttagcacctcctcagcacaaactggcccttgaggaatgaaataccgccgccggcacacacgctcctgagttaagcctttgtcaatgaaatgaacacccacttaaaaggaataacctgtccaggcacgatggaacattgagtaaccccttattctaaattcctggtccctgtaagactccttccccatgcccttgcccttttctgaccttcccctaaagtccttgaggcttaagcgggcatagtctgcagcaaacactggggaagctgagtccagacttcagagcacaggctttggatctaggccagctggatttgaacctcacatttgtgatcagctggcatgactgtttccaaaaagtccattttaatcctctacgtgaccctctgtaaaatgggatactgaatggtgagctagcacgattttacagagagtgaattttttttgtgtgtgtgtgaggcagtcttactctgttgcccaggctggagtgcagtggtgcagtctcggcccactgaaacctctgcctcccgggttcaagcgactgccatgcctcagcctcgagagtggctgggattacaagcatgcaccaccatgcccgggtaatttttgtatttttagttgagacagagtttcaccatgttggccaggccactcttgaacccctggcctcaagtgatccacctgccttggcctcccaaagtgctgggagtacaggcatgagccactgcacccagccttatagggttaaaatttaaaagaggtgatgctgttacaagcctgttttacaaaatgctcttataataaatcattatcatcactgttgctgtggttgtagcatcatcatcattaactcccagagggaggagggagtctcagagcaagctgctcaggggagactggatgtccatggattgtccagctcagtaccacttcctccaggaagtcctccctgataagtccagtcagcatcaccctctccttccaatgaaccccactagccttgtgatatcacagatattcttagttgacaggctcatggtgtagcctgtctagatcataagtacattttttttttttttggatcataagtatcttcaagaccaaaataattttctactcctgagcatgctcattggtcaaaggaaggaaggaatcataatagcgttaataaggctagcgtcttttcagaagttggttctttgtgccagtcttggtgctagacacaccgataggaagaatactccttcacatccccaggacaccaacatgggatacgtttgatcatcattcttaatttgcagaaggagaaataggctcagtgagatgaaatagccactccagtggcaaggctgggactggaagccgggcttgtcctgattccaaatccagtttctttccactgccacggagacggagagaagggacagtggccccagatggggatggggtgactggatgtgggcaggcctgcgggggaagagtgccctctgttgagcatccgaatgatggcagcagaaaagaagactgggcagaatcccagttatcagatcccctgagggaacagtcaccccgatcaccctcagtcagatgagtgtgtgtagatcaatgcctcatagatgaaggcactgaggcacagagtggttaagtcatctgccagaccacatggctcagggtgcagaggccaccttaacgggagaagagatggtcactccactctgcagcatcagcgcccaggtgggtagaaatcttgtcttctattcccacagaaagtaggtgcccaacagtgtttgttgaaagaatgaatgaatgaatgaatgaatgaatgaatgagtgagaggcatccttccttctcagtcgtcctggctctccctctctcccccagtattcggctggccaccatgagtgctttgtcggtgctgatctcagtggatgctgtcttggggaaggtcaacttggcgcagttggtggtgatggtgctggtggaggtgacagctttaggcaacctgaggatggtcatcagtaatatcttcaacgtgagtcatggtgctgggaggagggacctgggagaaaagggccaaaagctccatttggtggggtttccagggttttgaaaaataaagacaacctgtaatcccagctacttgggaggttgaggagggaagatcacttgaggccaggagtttgagaccagcctgggcatcatagcaagatcctcatctctaaaaagtaattttttctaaattatccagttgtggtggcatgcacctgtagtctcagttactcaggaggctgaggtgtgagttggaaggattgtttgagcccaggagttagggaccgagctgggcaacatagcaagacctcatctctaaataaataggtaggtggatagacagatagatagatagacagacagacagacagacagacaggctgggtacagtggctcacacctgtaatcccagcactttgggaggccaaggagggcagatcacctgaggtcaggagttcaagaccagcctggtcaacatgggggaacctcatctctactaaaaatacaaaatttagctgggcatggtggcaggcgcctgtaatcccagctactcaggaggctgaggcaagagaatcgcttgaacccgagaggtggaggttgcagtgaaccgagatcgcgccattgcactgcagcctgggggacaagagcaagacttcatctcaaatttaaaataaagaaaaaagaaaagaaaagattgatagatagatagatatccaaatgagtttacaaaaatgtggtctgtgcaaatgtttaaacacaacaaaccaatgcctttaactactacagtataatcctgtaggattgtgctattcatgatataattatggttatataaaagtaattaattctcagagcctcaccagcagtgggtccagcaagtttgtacagccagcatcttctttcagtcagtgcgtgtcagtaactgcatatgtcctctcattgggagagcctgtcgaaagtctaaatttgaaggcagctgtgaaggtaaggccaatccaaatggctctcccagatcctctgctgtaaccctgaccctgagtgaggacatagccaaccttcccatctcataggtgagaaagctgatgcctggagaggggaagggactgcccaagatcacatagcaagatagtggcagaacccaagcgagaacccacagttccagcctggcttagaagaaagtgcactggacttggagtcaaaggctggggtttgcatcccagctctgccataaatccctgtgtgactctgggcaatttaacctcttagagctttagtttcttcatctgtaatatgagggtagcagtactaccacatagggttttgagggagtaattgaattaatcacatgagatgatgcatgtttacaaaaaaaagcatgaagcccctttactgtgcctcagtgtcccaaaggactttggattttactctgagaaatacagggagaactagggagtgttgggcagaggagagccatgatctgacttatgttttaagatactctggcttctgggttcagaaaagactgaaggggcaagagaggaagcaggtggagaccagagcggcagtgattgccatcatccagactcagactaggacaatagctgtgagagtgatgggaagtggttggatcctgactgtattttaatagcagaattgacaggatttgctgatagactgcacgtggggtgggagagggtcaagatgacttcaaggttctcatctggcacaactcagcggctgctggtgccatttactgagatggggaatgttggggtgggatagatctgggagggaaaacccagagttcagtgtcgaatgtggtagcgttagggttaaggttgggggagggggggtagagatgtgtatgaaacatcccagtggagacactgaatggagatgtacaagtctgaagcttagtggaaaggttagggctagggatataaatttgggagttgttacaatacagatggtgtttaaagccatgagacccaaggagatcactcaggagtgaggataaagagagatgggaagaagtctgaggactgagtcctagaacaccctgcattttagaggggggacatgtgtaagagccagcaaaggagacagaattgtgcttggagaggcaggaggaagcccaggagagcgtgaggtcctggaaggcaaggaaagagagggccccaggtgggctgaatgctgctgagaggtcaagtcggatgagggctgggaagtagccattggatttggccaggagaccttggcatgcatggttgtagaggaggatgaaggcaacagcctggcttgactgattcaagagcaggagatgagaaagtggagacagcatgcaggggcagctctgccaaggactttgctataaaggggaacagagaaatggaggagaagcaggagggcaataatccgatagagaggaaaaatctgatgatacagaagagagatgaactgcaagagtcaagcctttgagttggaaagcaggagtgggattttgagcactgatacctttaggccgatgcagggacagttcatcttttttttttttttatacaacattttatttaaaaaaattattttcatagaatacattttcacattagagattcccattgtgcggaaataacaatttattacttatagttttatatttgtggacagattgttttagaacaagtagaatacatttgagaattaaatctcagtttacaatggataatattttgatatgtctctggggaaacttgcccttaaatggaacttctgtatcttcagaagcactccaagcgtttcttcctaggatttagaaatttataatatgagatagcagcatttcctaattttaaaatttccctagtatatgtaaccatcagtaggtggtatctactgactagagagggaagtttttgaaaattaaacactgtctaattttctgcaaagtttttattcatgaattaagagtatttccctttgtccattattcccaaggcaaatatggaaatttgatcatgtactaatcataataaagctggattctctttaagagattgagaaattaaaaggcaaaagctgatatatcatgtttagttatattgtgagtcttataagaagctgggaggcaaccccattaactcaccagaatacagaactcagtctcacaacttagatataattcctctcaaaccttttcctcaaagattaaattctgaaaataatcttgtgattaagagaagaaggctgtccaccaatgggcttatctgttatttcttccttattgtgagcttaatggcatgacaaagcagaggcaaagaggcatacatcaattcttcaaagtaggaagtcaaaaaggtcagagcttccacagcatggcaacagctttgcagatgcccacatcgtgatagttgaaatagcaaagcccagcaaaggttaaagctgaaaatgccaaaagccctgccttggcagctttctgcgaggcatccccatgaacataatcagtaacaacttgtccaaggccccagtgaccatgaagagtgagggctgcagccagggaatagtccgtcgcagagcaaggattcaaataagcagccggaagcagacccgggagcaaaacactgacaaccctctcgctagtccagtggagagatgcagccttggagccagaatggtggctcggtgacaagtgtatgtgctgcactccacaccattctgggataggtcggtcctgaagaaatgctgagatatgagcaggtctgaccactggagttcgcagcaacagagctcggcctccttgggcaccgcaaacggcactcagcctccagggaaccgccatctcgttcctgaggcggagagttcatcttaacgagagaaatggcagggactgtgaataggccggcagatttggtggcgggtgccacaggttcagtctcctgcagggagaggagaaaatgccttactaattccttgtattttctcagagaaacaagaggcaccgtcatcagcctcatgtgagggtgggaaggagggatggggtttgcggagagggaaagtgtggtatggtcatctgtgggagtggaagagagtgagagggctgcaggggtgcagcgggactgcaggctggcaccagggtccctagggcttgtagttggtggaaagtgcatcagtgaccagggctgtgtgcagctgctccaggcaggtgtggaagaagcagagttgaacttgcccagcctggagtgctgcccagagtgagcccaaagcccaggggagaccagagatggggctgtttgcaaaggaggaagtataacagtagcccacaaaatctgagctggttaagaaaggagagagagtgaaaatggggagcccagcctggcagcctgggtacacatctcagctcaacccacactagctgaatccatttgggccccttcgttgacctctctgtgcctcagtttccctatctatagaatggggataagaataaggctacttcctagggctgttgtgaggattgaacaagtgaccgaacacttgttcaattttgaacactgttctaaagcatttaggacagtgcctggcatggggtaagtgttgcggcagtgctgttattttcatcatcaccattgttctcaggctgcgttgattggagctgctgaagggaggcaatttaaggaagtgagccggacagataggaggtggtggtggttatcaggtgcgatgcttgaaactgaggcttcggaggcaacagttactggtaatgacaaggtctaaggcttgacagtgggtggcagaagtgtaacgcagggaaagagacgagcggtcaaggagccgagagggaaggagttgggtggactaagatcatttgtggaagaatgatggagagaaaggctgaagggcaggggctgacatcatcagtgaccaagaggcggccgggaggctgagaccacagcaagaaagggagagtgtgatggcatcttcttcaagggagctggggatgtttggggtggaaaaaagaacaatggtctgggagggaatatgggaaatttttttttttttttttttttttttttttgagatggagtttcgctgttgtcatccaggctggattgcaatgttgcaatcttggctcactgcaacttctgccttccaggttcaagtgattctcctgtctcagcttcccgagtagctgagattacaggcacacaccaccacgcctggcttacttttgtatttttagtagagacggagttttgccatgttggccaggctggtctcaaactcctgacctcaggtgatccacccgccttggcctcccaaagtgctgggattagaggtgtgagccaccgcgcccagcctggaagtttgtatttattaatttttggttgtcttcatctgtgtatgtgactttaacccctaaatacttcagtgtacatttctttttttttttttctttgagacagagtcttgctccatcaatcacccaggctggagtgcggtggtgtgatctcggctcactgcaacctccgcctcctggattcaagcaattcttgtgcctcaccctcccgagtagctgggattaggggcatgccaccatgcccagttaatttttgtatttttagtagagatggagtttcaccatattggccaggctggtcttgagctcctggcctcagttgatccacctgtctcagcctcccaaattgctgagattacaggcgtgggccaccataaccggcctcagtgtatatttctgatgcagttgggttctgtatccccctccaatctcatctcgaattgtaatccccacgtgttgagggcatgacctcgtgggaggtgattggatcacaggggtggtttcccccatgctgttcttgtgacagtgagtgggttttcaggagagctgatggtttgaaagtgtggcacttcctctctctctttctctctctctctcacctgacaccacgtaagatgtgccttgcttccctttcaccttccaccatgattgtaagtttcctgaggcctccccggccatgccaaactgtgagtcaattcagcctcttttgtttataaattacgcagtctcaggaagtatctttatagcagtgtgaaaacagactaacacaatttcctaaaacaaggggacattctcttacataaccttttttcagttaacaaaaatgagaaattgacattgatatattatgattaccttattctcatttcaccaattttctcaataatatcttttctagaaaaaaatatatattttttgtggtcgaggattacatcttgcatttagttctcatgtcttattaaattccatcaatctggagcagtttcttcatctttctttatctttcatgaccttgacatgttttgaagtttcgagccagttcttttgtagaatgtgggtttgtctgctgttcctcatgattagattgtgggtatgcatttttggtaggaattctccaagagccgtgtgtgcccttcttagtatatcatatcagaagacatgctatcaatttgccccattactgggtgtgttaactgtgatcattgggttaagatggtacctgccaggatcttccactgcaaagttactattttcccctttgtaattaataaacatcttgtgaggagataatttcctatagaaatcctgttgatcatccaactttcacccactgattttagtgttcattgattcttccctgaataaattagtactataataattgccaatggtggttttctaattccatctttccttcagtagttggcattcttctgtaaggaaaagctttcgcttctctgttcatccactcatctatgtacttatttatatcaccatgggctcctggattccggtttacacacttccattttctgccttttctctctgcttaatataaggattaatgagaactccctgattcccaggaagaaaatgtcagcagagctttcttaggcggaatgaagagaattcagtgtaagaaccataaaggtgtatctgtgtagtatggacagttttaaaaaacaaacaaacacaaagaacctccaagggcaggaggtgctgccagactcaggagggcactagaactggctatgagaagccactgagatcccaggtagtctgtgctctccatcttttggctcttattctctccgtacatctaacatctctgtacaccagctttctctttagcgaaaaacgtgtcccctccacccacccatccacctccacttgttcctgcatttctatgtcccagatcctgcagaaaacaactcttttctctcagttagtctcaattctgtagtccagggagagagaatctgatcagtcccctgggtcatttttccactctggtccaagcagctacagctggcatgggaaatagttcacacagtaaaaacatggctgtcaagaagaggagtaaatttcagaggcagaacactccctgtgagcccgaacctcttcctgctttgttgcagtcttcataacgattgctttaaaagactgcattgatataacatcatctctcttctctgcatctttgacttgctagcttaactggtctagaggagggcttagcactgattttgagtattcattttcctcaaaacttcaattcagcctgggtttcttcagcaggagggcccgggggaaccagagccagggaccagagtcatttcagtgcaccagctcaagaaatgaatattccaggccaagaatccccaagtgttcttcctgaactccttcctggtggagttcaaagagatgaaaaacacaagcccgcttttcagttcttatcaggaaactgcatagactttcctctttatgtatgactgagggctttttaccatcatttgttcccttcacaaatatttatttggtatttactatataccagggactcttgtggcagtggaaaatacaactctcatggaacgtctgttccagaaggaaagactgccaataaacaataaaataggcaaaagatatagcatgttagagagtggtaagtaccacagataaaaatgaaatggagaaaagaaacacgaaaagttggggagagaggataactgtttgagagggtggccaggggcagcttcatcttatcaagagggtgattttttgagtacagacctgaaggtaacgagtgcacaagccatatgggtacctgagaacagcggcagaacaatggcagggtgctgggagggctgtttaccagccacgctgtttagaattgtcagcacatggtgataaaaaaaaaaaaaaaaaaaaaaaaacaggctgggagcagtggctcatgcctgtaatcccagcgctttgggaggccaaggcggatggatcacttgaggtcaggagttcgagaccaggctggggaacatggtgaaaccccgtctctactaaaaatacaaaaattagccgggcacggtggtgggtgcctgtaatcccagctacttgggaggctgaagcaggagaatcgcttgaacccaacgggtggaggttgcagtgagccaagatggcaccagtgcactctagcctggcgacagagtgagactccgtctcaaaaataaataaataaataaatacaaataaaaagcagacagactttttagttggctttagaattcttagacaccctctacagacaaggcaccccgattgcttgcacccagggtggactactccctccaccctgcccttgttacaccctggctgggggtcagcatttcaggcagctgaatgacccaaagtgggaacacgctagtgggtttgaggatgagcaagtggaggagggcaataggaggtgacgcccgagaggtcaggtgagagtggatcctgcagggtcgtggcaagaacctggaccttgactttgagtgacatgggagccgctggaggcttctgagcagaggagtaacatgatctgacttgcattttattttatttatttatttgacgcagtgtcactctgtcgctgaagctggagtgcagtggcgacatctcagctcactatagcctccgcctcccaggttccagtgaatctcctgcatcagcctcccaggtagataggattacaagcaagcatcaccacgcctggctaatttttgtatttttagtagagacagggttttgccatgttggccaggctggtatcgaactcctgacctcaggtgatccacccacctcagcctcccaaagtgctgggattacaggcaaaattagaatatatctagaatttcctgaagaccttagtttggtattataagaagtctggttgcttcatgttgcaaaatttatatcactcatcactcccgcagagttaaaattccgctgagaagtaggaatcagtgaggtgcgtgtccatgtgggtttttgccacacctaagtgaaccttggtcaaaagcatataagagctactgataggccgggtgtggtggctcatgcctgtaatctcagcactttgggagggaaggatctcttgagcccaggagttcaagaccagcctgagcaacatagcaagattccatctttacacaaaatttaaaaattggccaggcatggttgtacattcctgtaatcccagctactcaggaggctgaggtgggaggattgcttgagcctgggagttggagactacagtgagctgtggccacaccactgcactccagcttgagcaatggagcaagactctgtctcaaaaaaaaaaaaaaaaggccaggcgcagtggctcatgcctgtaatcccagcactttgggaggccgaggcgggtggatcgcctgaggtcaggagtttgagaccagcctggcaaacacggtgaaaccccatctctactaaaaatacaaaattagcccagcgtagtggcgcatgcctgtaatcccagctactagggaagctgaggcaggagaatcgcgtgaacctgggaggcaaatgttccagtgagccgagatcgtgccattgcactccagcctgggcagagcctgctgggttgggctgggtaagctctgaacaccagtctcatggcttcaagtcacacctcctaagtgaagctctgaactttctccaaggactatcagggcttgccccgggcagaggatgccgacactcactgctcttactgggttttattgcagacagactaccacatgaacatgatgcacatctacgtgttcgcagcctattttgggctgtctgtggcctggtgcctgccaaagcctctacccgagggaacggaggataaagatcagacagcaacgatacccagtttgtctgccatgctgggtaaggacaaggtggggtgagtggtctcctacttgggctgagcagaatggctcagaaaaggctctggctgaaaaaatctccctcctttaccaagttcccctgggtgtctgaagcccttccatcatgattcatttctttgagtagtgtttgctaaattcatacctttgaattaagcacttcacagagcaggttcaggaggcctggggtatgcagatttcaaccctcttggcctttgtttccttgtctgtaaaatgtggttagctggtatcagcttgagagctcggaggggagacgtgacttccccatctaactctaagtgacaaggctgagactctccagccctaggattctcatccaaaacccctcgaggctcagacctttggagcaggagtgtgattctggccaaccaccctctctggcccccaggcgccctcttcttgtggatgttctggccaagtttcaactctgctctgctgagaagtccaatcgaaaggaagaatgccgtgttcaacacctactatgctgtagcagtcagcgtggtgacagccatctcagggtcatccttggctcacccccaagggaagatcagcaaggtgagcagggcgctgcccttgggcagcacttgggtctaacaggactagcacacatatttatgcccctccccaccccagggccagcgtgggttgggagagggcatgccgggtggtggagctgtgcctgcctctacagtggagctctaggtagaatgctgggtggtcacagtgggcctgggactcaggagactgtccagtgatcaaaggctttctgggggtagtgattaaatccatccatgctaacatgaaacagacctcagtttgaaccccatttctgctagttgctaaagtcagtcaccatgagcgagagtcagcagcaacagactagactagaattagccagcctctctcttccccccaacaaatttcaagaatggaaccatcagaatcagaagtagagaagtatgtgacactagccatgtggctctggtcaagccacttcaacgttttgagtctcagtggcctcatctgtaaagtgggaattaagagatggtgcatgtaaagtgcttaacggggagtaaatggtaggcaaacattagctgctgctattagtaaagagagacgatggtgtgtgtgagtcttgtgggcagagatgggtgagaggggagacaaaacaagttctcatgatgatgggggaaggggctccagctggtggtgtcggagggaagtctggacagaccagtggtggggctcgggtgggaggcactgggggggctggagtggaaagaatgtggccacagatgacagcttcacagcagaattcagtgctaagaggaagtgagtggccatgagttccatggtgacagaaagtctaagacacccagcaaggcaggagtgggtgtcaactcagggaagcccagaggctaatcctaggtgagagctgagggtgtcagataagagcaaggcaaggctccggttctggagcagtgaaggacatagcagagctatgacccaggaacaaggcccagcttattgaaactgggcccagtcacacagggtggcacaggcaccaagtagccaataataataataaaaacaataacaatgatttgtgtctactgggcatttattcatgttctatgccagacactgggctaagagctttatatgtggaaactcatttaatccttacaataaccttatgaagaaggtacatccaaaaccccattcttctaggccaggtgcagtggctcacacctgtaatcccaatattttgggaggctgaggcaagaggattggttgaggccaggagttcaagaccagcccaggcaacatagcaagaccctgtctctaaaaaataaaacaaaaacccattcttcccgctgcccagggacacaccactaatgagtgtgatgggtgcctaggatgctgagcacctggacttcccagctcattccctaaatgctgcacaatcagggtaactgtgccctgagcctaagaggcagtagtgagctggcccatcatgtccactgatgaaggacacgtagccccaacacaggggagaagtggtttcaggatcagcaaagcagggaggatgttacagggttgccttgttcccagcgtgctggtcacttgcagcaagatggtgttctctctctaccttgcttcctttacccacacgctatttctttgcagacttatgtgcacagtgcggtgttggcaggaggcgtggctgtgggtacctcgtgtcacctgatcccttctccgtggcttgccatggtgctgggtcttgtggctgggctgatctccgtcgggggagccaagtacctgccggtaagaaactagacaactaacctcctctgctttggctgaaggccagcaggacgctgggacctgatgggccactgtgcagtgcacagctgcattaggcaggtgtcggcgcattctcttattggcttcaacgcctagtgagggatccatcctggctcggtggcgcatttgttaagatgctcgggagcaggtggcagaacccatttgagcttgcttgggcattggggagaatttgttatcaggctactggggtgtcacagaactcaaggacagggactggagtgttgtggggagccccgaagcccctgttttacttctttctttgcttttcctgaatatctgctttattcttactctatagacatgcttcctcctctttcaccccacattgtggggtgtagtcttttgcttcaagaaagcagcctggtggatggaatctcttggccccaatcccaaattctctggagaaggggctctttggtttaacttggataatgttgtcttcagctgggggtgggcacatcgtgcatatgtggctgctgccggggaaccacgtggatgatgtgagaggagcagcacccagaagagggagtgctgggctgatggtccaggtcgtgtccacttctgattgtttaattcttcttctaagtggatggatctttctccaatactcagcaaatcctgatcgttccagaatacttcattatagccaattggttataatgtgcttctctaagagaaatatttagggacaacaaatcttcatgggtttgaagacttgatggaggaaaaaggagtagattttcgaaggctggatttggatgaacaggggctattcagggagtgcattccaacctaaaattaggaaaaactggctgggcgcagtggctcacgcgctttgggaggccgaggcgggcagatggcctgaggtcaggagttcaagaccagcctggccaacatggtgaaacccatctctactaaaagtacaaaaattagccaggcatggtggcgggcacctgtcatcttagcgactcaggaggctgagacacgagaatcacttgaacctgggagacagagcttgcagtgagctgaaatcgtgccatggcactccagcctgggcgacagaacaagactctgtcttaaaaaaaaaaaaagtggtttatatacagagtggaatattatttagccataaaaagaatgaaatcctgtcatttgcagcaacatggatggaactggaggtaattaaaaaataaaattaaataaggaaaaacgtatcaatacttcgattaaccaaaaccagggcaaatctgattttcatctttgcaaggggaacaaatttcttttatctcctctggctttgaaaccctgaaatgaaaggaggaagggcagaaaaaagaacacatagcaagttatcatcagtctcagcgcccatcgcattccctgagcttgtttccttgacttcatcactggcaggactattcaaaaatgattcgctcattcattcatatattcattcattcatcattccttcattcaacacatacgttttaacactcatcttgcttttcaagctatagtttagtgagcgaaatggatacacacaatacagtgtgagaacagcaagagggcacatctgagctagcctgggatgggtctggaaatgcttcctggagcagaggaaacggttgacagccaagtgttgacagagaagtagtattagccaggcagagacatggggaatgtattccaggcagaaggcacagtgtgtatgaaagcttattgttaagaagagtgtgtggcccaaccaggaaacagacattctaaaggcatagggtccacccaggagcatggtggacccagatccctgaaagatgggaggtgctcaggcacacttcctgggctagttgaggagtctggatatttatttatttatttatttatttatttatttatttattgagacagagtctcattctgtcacccaggctggagtgcagtggtgcaatctcagctcactgcaacctccacctcctgggttcaagtgattctcctacctcagcctcctgagtagctgggattacaggtgcccaccaccatgcctggctaattttcgtgtgtgtatgtattttgttgttgttgttgttgttgttgttgttgttgttgagacggtgtctcgctcttttgcccaggctggagtgcagtggcgccatctcagcttactgcaagctccgcctcccgggttcacaccattctcctgcctcagcctcctgagtagctgggtctacaggcgcccaccaccacgcccagctaattttttgtgtttttagtagagacggggtttcaccatgttggccctgctggtcttgaactcccgacttcaggtgatccacccatgtcggcctcccaaagtgctgggattacaggcatgagccaccgtgcccaacctggatttttattctgaagactaatagggattctaaggaaggaaccagcctgattgaatttgcatatgtgtccacatctgctggctcacggctgtgtgggaggctgagtgatggggaggaaggattactgagtagggatctgaaggtgtggcctcatgctttctttctaaccagctgtgttgtctttgggatggtgcttaaatttgggctagaccagtgggtcttggtcaccccccaggggacatcttacaatgtctggaggcgttcttggttgacacagtggggtgagggctgctactggcagctcgtggggagagaccagggatgctgcttaacatcctacagtacacagggcagcccccaccacaaggaattatcagctgaaattgtgaacagtgtctacactagacccttgctactcatagtgtggtccgtagaccagcagcattggcatcacctgggaccttgttagaaatgctgttagaccccaccccacatccactaaagccagctcttcatttcaacaaactccccgatgatgtgagtgcacattcaagtctgagaagggcttctttgaggtgagccttagtgcccatccccctttggtggccccggataccaagggtgtgtgaaaggggtgggtagggaatatgggtctcacctgccaatctgcttataataacacttgtccacaggggtgttgtaaccgagtgctggggattccccacagctccatcatgggctacaacttcagcttgctgggtctgcttggagagatcatctacattgtgctgctggtgcttgataccgtcggagccggcaatggcatgtgggtcactgggcttaccccccatccccttaacactcccctccaactcaggaagaaatgtgtgcagagtccttagctggggcgtgtgcactcggggccaggtgctcagtaggcttcggtgaatatttgttggctgatttattcagaaattctgtccagcccctaccttggatggatttatcacctctccaggccacctcttctttccaaatagggccacctaggtatagaccaaagacacgaaatcttttgtgatcccacaaacacagagcaggtcaaataggcccaagccaattgagactgtggttcaggtcgtgatgcagagctttgctgtggacgtgctcccactgcgtactagctgggcatgtggcttaacctttctcagcctcagtcgccccattgtaaatggagataatgatactatctcccctcacaggactgttgggatgctactggatttaataagctaatgcagggacatgctaagcacaacccatccctgaggcccagagaggggtgggccttggctgaggtctcactgcgaggtgggaatgtgggcctccagaccagaggtaggtcctgtggcccctagacagtggacagcaatggtcagtttgacacaccagagccctagccattacttcctggatgttgtgtgaatattttctggacatggcttatataaaatgaaaaagtgaattgggcacgatacagggatagatttttagagatgaactggtagcatgatgataatcatattcactgataacatttactactgttattgactgctttaaaagtgttgggcattgtgctagaaaccattatatgcattatctccttgaattctcacaaccgcctactgaggtattctcagactctaagaaatgagatttaagagaagttatctgcccaaggtcactcggctggaacctggctgtaaaaatggctgaagcaggtgatgaggagctgatgcgtttggacgtgtctcagagaaatcatggaggcgctgcggttcctaccggttcttggatgccttctacagagacaaccatagccccaaattatagggatcacatatcagtgggtgagacatccttgcttgggatgaggaggggatgagctgtgtgaagcaaggcgcctctgtgatgggttccagtgatgtgtctgccactgtcttaataactgtgcaattctaagcagaacctttcctgtctctgggcctgagagttcccctctgaaagatgaggacttgacctagcaaggtcctactcacatgcctgtagagaacaggcaggggaagttagaaaaaaaaaaaagccagtgaaggaagggagctcttcagcttgcacccatcatcacagtgcagggacccaggctcagtgttgccagatccaatgacttctcaagagctcaaaatctagagttttgcatgtgctctcccaagtactggcagaaaattcaagattgttagtaacactgtgtggctaaattctgcttgtgggctgcctagattcccaattctgtgattctgtggttctctggaagcattggttctccacagcacctgcatcacttggaaacttgttagaaatgcaagccctacctacggccccaccccagacctacccagttagaaatctgggggtgggacctatcagtccatgtttgaacaagccccacaagtgttctcttgcaagctcaagttttagaaccactgacctatagccaaaaaagaaaaagccaatcagtggttttctggtaaaggattaacttaacaaactggctttccaagaaaataaagccttgattggtagcacttgcaatttctatggtacaaacgcttcccgcatgactgagttcaagctgtcaaggagacatcactatacatggacttgggaagagatgagaacaatcagcccactgagcctatgggaactggctccagcacatccctgcaagtcaactctcatcagggtgagtgagttgaggaccaagaagcagttatcctcttgcctttgcaggacccaggcaaagggaagggcatagtgacagtgatgatctctcttccggaagtctttggtttgctgagagtaaaaggcgtgggcttcaccagtggtgaagccagtcatgcagccttagtcctggtactgaaactctctaaatctcagttttctatctgtaaaatgggaaaataagacctatgtcacagggttgctgtgcagatttagcaacagaacatagccccgttctttatgatgactgatgctgcatccgtatgaggacatctctatgtaatggaaagatggagagaggattaagcgcaaagtcacaacacttaatgggaactgtggattagctacttggtggcattgggcaagtcagttgactttgcattaattccacaaacaatatttcccaatttcctattcagatgagcatatgtgattgagtcagatgctgtgatcagaaccaggatggagcatttcccacaaactgtgggatttttaagtaatgggaaggcacactgaaatggcactgaatcatgcagttgcagatactctttttcaattctcagtcctttgattacgtcagggagaaaagaaagtccccacttggcctgagaatctctgcacccttctagctcttgttaaccactcttttgaatagcagagaaaacctcagactgccatatctgggagagattttagcaacattttgttttcattgtatctctttttacagctacctcccatttcccttctatttcaagctagtaactcagttttcttttaaattcaattatttaaatgtaaaaataagtctatttggagaaaaaaaattttaatagcatctctggaatgccagtatggctaaattcatgaatgttgtcctcaaatgctgaaatctgggaagcatctggccaagctttgtggacaggcctgcctagtttgaatcccaagagccacccagtccaagccacaaaacattggaattcttggttcacttccctaacctgaacttgccctctgtgaaatagggacactaatagctcactcacagggctgctgtgaggacatgtgttgagctgagggtctcgccaggggagaccctgtgcagggagactgttatcatggtgatggatttctgcttcattcatttctttttccagacagcatcatatagaatgagttgtggggtggcagtcagcaggtttgggtttatcctctattctgccacttattacttaaaaaaaccccaaaaaacccaacttatatagtataagctatatccagaaaagtgcaaatatcatacaagtaccatttgatgaatcttctgatatccccacataaccaacacccagaacctcttcttgtctcattccaggataaccactaacctgacttctaacagcatcagtcagttttgtctgtttttgtacattatatatgtgatggtttgaatgtgtcccccaaatttcatgtgctggaaacttaatccttcaattcatatgttgatggtttttggaggaagggcctttgggaagtaattaggattagataaggtcatggggtgaggtatgatggcactggtgacttataagaagagaaagagaaatctgagctggcatgctcttgccctctcactgtgtgatgacttctccatgtcatgatgcagcaagaaggccctcaccagatggtggcaccatgcttttggacttcccagcctctagaactgtgagctaaatcaatttattttctttataatcacccagtttgatattttgtcatagcaacagaatatggacaaagaaagaaaattaatgcaagaagtagagtttttactgtaacagattcctgaaaatgtggaagtggctttggaactgggtgatgggaataggttggaagagttttgaggagcaggctagaaaaagcctgtattgtcaagaatggagcattatgccaggcacggtgtctcaggcttataatcccagcactttgggaggccaaagcaggtggatcacctgaggtcaggagttcgagaccagcctagctaacatggtgaaacgctgtttctaccaaaaatacaaaaaattagctgggcgtggtggcgcacacctgtaatctcagctactcaggaggctgaagcaggagaatcacttgaacccaggaggcagaggttgcagtgagctgagatcgtgctattgcactccagcttgggcaacaagagcaaaactccatctcaaaaaaaaaaaaaaagaaagaaaaagaatggagcattaaagacagttctgcagttctggtgagggcttaaaggaagaccccagaactagggaaagtctggaacttcttaatggttactgaagtcgttgagatcagagtgctgatagaaatatggctggtaaaggccattctgatgaggtctcagatagaactgaagaaccacgtgttggaaactggagcaaaggtcatcctttttataaagaagcaaagatcttagctgaactttttctgtgccagagtcatttatggaaggcagaaaatctgtaggtcagccatgttgtagggaatgaaagaacattttcagctgagaacactgagagtgtgacacaactaccgactgataagaaaactagtacacataaattagccaggcgtggtggtgggcgcctgtattcccagctacctgggaggctgaggcaggagaatggcatgaacccgggaggcagagcttgcagtgagccaagatcgcgccactgcactccagcctgggcgacagagcaaaactccgtctcaaaaagaaaaaaaaaaggaagaaagaaaattagtacacatagaacaaagccagaggctgttcatcaggacaagggagaaaaactccaaagccatttcagagatcttcaagactgcccctcccattactggcccagagctctaagagggcagaatggtttggaatgaccagctgctgcccagggctgccttgggtctctgctccccacatttctggtgcagcattcctcagccatcccagctgtggttcaggtggccacaggtgtgatgtggaaggtaaaagtcataaaccttggcagcatacacatggcactaattttgcaggtgtgcagaatgcaaaagctgagggggcatgccttcttccacctacatttcaaagggtgctgtgaacagccaccccagagagcccctagtagagcagggtctagtggagctacaagggtggggccaccgccaagaccccagaatggtagagctatcatagtgcaatgccagcttgggagaactgcaggcatgagactccaacctgtgcgaagtgcaacatgggcagaacccagcaaaaccacaggggcagagctccccgaagcttcgggggtccaaattccatagtgtgtccaggaggtggcacacagagtaaaagatcattctgaaggtttaaggtttaatgttgttttctatgttgggttttgtactttcctggaaccagttaccctttttcccttgcctctttttccttttagaatgggaatgtctgtcctatgcctgttccactgttgtattttggaagtcaataacttgttttgactttacaggcttacagccagagggaatctcccatagaatgaattgtaccttaagtctcacccacatctgatttagatgagaccatggactttggaattttgagttggtgctggaacaagttaagactttgggggttgtctaagtgtggtgtttcatgcctgtaatcccagtgatttgggaggttgaggtgggaggattgcttgagcccaggagctcaagaccagcctgggcaacatagtgagacctgtctctacaaaaaataaaaataaaaaaattagccaggtattgtggcatatacctgtaattctagctactcaggaggctgaggtgagaggatcacttgagcccaggagtttgaggctgcagtgagctatggtcgtgccactgcattccagccagggcaacagagtgagactctgtctctacaaataaaattaaataaacttagctggatatggtggcacacatctgtagtcctagctactcaggaggctgagacaggaggattacttgagccaaggagtttgaggctgcagtgagctatgatcatgccactgcattccagcctggatgatagagcaaaatcccatctctaaaaaaaaaaaaaaaaaaaaaaaaaaaaaaaaaaactttagtgctattggaatgaattttgcatgtaagaaggacatgcattttgggggctggggcaggatgctgtggtttgaatgcatccctcaaatttcatgtgttggaaacttaatctccaaattcatatgttgatgaaattggaggtgaagcctttgggaggtaactaggattagataaagtcatcagggtggggcccctatgatgagactggtggcttacaagaggaagagagaactgagctgacatgctcttgccctcttgccatgtgataccctctgccatgtaatggcaggcacagcaagaaggtcctcaacagatgccagcagcatgttcttggacttcccagcctccagaaccatgagctatatatacttattttacaaattacccattctgtggtattctgttatagcaatagaaaatgaactgagataatatacatggaatcatacagtaagtctgtgcttttgtatgcttcttttactcaacattgtagttgtgagattcatccaggttgttaagcattgctgtaccctttttccactgggatatagtgttctgtcatgcttgggtcttaatttataaaggtgactgagtggcattttcttccagtattattggaaggaaagttttgttgttcacagttcccctgtaaacaagaggcagaacacgtcatgcagggccacacaaaactgtatcatccagggaccaggcagcagaaagagagggggaactgggactatgcctttatgaaaaagagtggtgggagagtaactgggtgagggcatccactaatgggcaggaagtgaaaacacatatgttagaatttgtagctgaggggtttataatatgagtttcctatgcctgagaaagctgacttgcaagaaaatgagataaacaactttggccattagtgtggccctgtcataaatgaatgccagataggcaaatagagaatctaagaaaagatagttggaacaagtgttccattgtgtgaatgcagcagaatttatttatccattattgaggaggatttgggtagtttccagtttggagctattatgaatattctagtattgctcctatgaacattctagcacttttatttttggagcacacgaatgcacttctgttgattatatgcctagaagtgaaattgttgaattatacagtattcacacagtcagctttagtggctactgctaaacaattttctctagtagtttgcgccaatctaatcaccagtagtgtatagaagctccttttactccacattttgccaacacttggtgttttccttctttttgattagtcatttagcaatcaaacctattgtttacattttgatatctccaataactaactaaatggagcacttttaatatgctttttggacagttgaatatcttttcttgtgaaatgtctattcaagttagtttgcccattttctattgtggtgttctgtctttttcttattgatttgtaggaattccttacgtatcctggatatgaatcccactttgtgcgttacctttttccttctttctttctttttgaaacagagtctccttctgtcacccaggctggaatgcagtggcgctatctcagcccactacaacctctgcctcccagcttcaagcaattctcatacttcatcctcctgagtagcttagattacaggcgcatgccaccatgcccagctaacttctgtatagacaaaataatttttggtagagacagggttttgccatgttggacaggctgatcttggactcctggcctcaactttggcccaccttggcctcccaaagtgccaggattacaggtgtgagccaccatgcccagcccaccttttactttcttaatggtgtcttttgaacaagagaggttcttaattttaatatagcccaatttatcattgttccctttatgtttagttcttttatgtcctttttaagaatttttgcagccagcgcggtggctcacacctgtaatcccagcactttgggaggctgaggctggcggatcacaaggtcaagagatcgagatcatcctggccaacatggtgaagccctgtgcctactaaaaatacaaaaaattagctgggcgttgtggctcttgcctgtagtctcagctactcgggaggctgagatcacgccactgcactccagcctggtgacacagcaagactccatctcaaaaaaaaattttttttgcaaggtcatgcatatgtccccctgatttttttcctaaaaatcacttattattagatcaatgaattgagtaattgactacatttttcagtcattcaacaaatatttccctgaggttttgataacctgaactgtgtttggagctggggaggaagcaaactattgaagatatacaaagatggcaaagatgagggcctggagcttgccacacggaaggggggatggctgcctgaatggttgggcaggtagttgttgacatctgcactccctacatgagcagcagggtggcaactctttttatctttttaatttatttttcttttctttctttctttttttttttttgagatggagtctcgctgtgttgcccaggctggagtgcagtggcgtgatctcagctcactgcaaactccacctcccaggttcacgccgttctcctgcctcagcctcctgagtagctgggactacaggcgcctgccaccactcccggctaatgttttgtatttttagtagagaaggggtttcactgtgttagccaggatggtctccatctcctgacctcatgatctgcccgcctcggcctcccaaagtgtggggattacaggtgtgagccaccacacccggccttaatttatttttctagtctgcaggtaattctttttaattctctccactctcctatgatcttatgaggtagggactgtcattatttctcccactttataatgaacaatcagtaaagacagggaagataaccaaatgacatacaaggtggggtccaccccatgaggctgcaggcttggagctttgctttgtcttaaaaatgagaacatgagctgcccacctgttgagacaagaaacaggaaaggcttaaaaaactggcttgttatgtacaactatccgtggggctgcagtgaacgggctggcagtgcccaggtgcaggctgaaccctgggacaatcacattcagcatccaagggcccccgtaatagcttaatgtttgaattgaacccctggggttgccttgaaggagagaggtcgtggaagtatgttcaaggggtagggatgggcaggggagatgggtctgaaagccaagctctaccccacccaccttgccccaagagaaatagaaccttcatctttaattgcctaacgagaaaactggggctggccagatgtggtggctcatgtctgtaatcccagcactttgggaggccgaggcgggcagatcacttgaggtcaggagttcgagatcaccctggtcaacatggtgaaaccccgtctctattaataatacaaaaattatccaggtatggtggcgcatgcctgtagtcccagctacttgaggcacaagaatcgcttgaacctgggggacagaggttgcagtgagccgaccactgcactccagtctggacgacagagtgagactccatctcacaaacaaaaacagaaaaaaaaaaaaaaaaaagagagagagagaaaactggaggctctgagaggttgagggacttgcccagggtcttgcagctagtaagtgacagagctgggacttgagcttgggttttctgactcctggtctggttcattatccatgaggtgctgggaactaaaataagccacaatcttggaatctccgtcgcctccctccctcccacatgtctgcgtggctttttgggaaaatgccaggggaatgtaccagccagggagaggacccttgttttcctcatggcccttcctggcaatggcactactgacaccgacagtcctttttgtccctgatgacctctgctgcctgatgcccaagtgaccacctctgctttgtcatttctaggattggcttccaggtcctcctcagcattggggaactcagcttggccatcgtgatagctctcacgtctggtctcctgacaggtcagtgtgaggccacctttcttccaccattgccaggacacagcacccacgtccagagcgcaccctgccgtgtggctggatgtctatgtgccccatctccttccctgaggatcacataatttcagaattggaaaggttcttagaggtcacctgctgctaatgtggactgtgaggccagggcagggaagggacatccctgaggttataagtagggtgagtggcaacgttgcagacttttgaacccagggctggtgatcacactcagttttgcacagaagcccgagaaaatccttacacccaaaagcctaccttttatttctgaggacacccataatactattttattcaacagatatttattcaatatccactatgagccaggcactggggacacagcagtgagcaaaacaaattccctgaccccatggaattgaccttctagtgggggaaggtattagcaataaatagacaaataagtgtctactacgccagatgggaagaagtggctgtgaagacagagcaaactagagaaacatagagtcaatgtgggatggggtgttcttttaggggggtggtcagggaaagcttatctgagtagttagcttttaagcagagaccccaatgaagaggagggagatatgcgatgcatttagttaggggaagaacattccatgaaaataggatagcaagtgcaaaggccctgagacagcagcatgctttgtgtgttgagggaacagtaaggagaccagtgtggttggtgtgaatggagtgagaaggagcagcaggggttgagggcagaatggtagtgaggagcaggcccttataaaagatgggaagccactggagatctttcaacaaaggggaaaagtatgtttctgttcttgcaataaaatagaacagcaaaaaatctaggggagttgctaattagccagttttacttatatgccaggtgaaaatatgtggctaggtgcagtggctcatacctgtaattgcagcagtttgggagaccgaagtgggcagatcatctgagatcaggattcaagaccagcatggccaacatggtgaaaccccatctctactaaaaattaaaaaataagccaggcgtggtgttggatcccagctacttgggaggctgaggcagtagaattgcttgaacccgggaggcagaggttgcagtgagccgagactctgtctaaaaaaaaagaaaaaaagaaaatacacattcaggccaggtgcagtggctcacgcctgtaatcccagcactttgggaggctgagacaggtagatcacttgaggtcaggagttcgagaccagcctgaccaacatggcaaaaccctgtctctaccagaaatacaaaaattagccaggcgtggtggcgtgtgcctgtagtcccagctactggggaggctgaagtaggggaatggcttgaccccaggaggtggaggttatagtgagtcgaggttgcaccactgccctccagcctaggtgacagagtgagactgtctcaaaaaaaaaagaaagaaaatatacattccatccagaactgttcacctttattctacaagcaaacatcttttattggttagacacccatatatgtgtccctaagcaggaggtgaatgccaaataagagacaaatggcgtaagacactatgagttgtgtgacgttgggcatgtcactttactccctctgagccttggttagcttctctgtaaaatgaaaggattatggtaactaagctggcttccttccagctttaacaaactgtatggaggtactttttggagttacctgggtaatttttgagtgtgagattggctagaattgctttaatataccatgtctggccttagctttttgcagagtctttgtgaagaagcagaggcggagtagcgttaattccgtaagttaacgttcagttcgtggcagctggcaatccaaccctgggaaaggctgccggatttagcaaaaatgcaaggtgtctgtttttaaatttgaaatgaattgggtatcctgcattttatttggcaaccctgtcctgggactcacactattcactgttatcactggtatgttcaaagtggtgctgacttgccctctgtcttgcaaagtaccaggaggtcttttcttattcttcactggagtcaaaaaagagaatagaggaaaagacaatcatattgttcctttaagagttaagaccaacaagttttcttctttacatgttgtttttgacatgagcaaactggtgattaaaaacaacttgggtggctcatacttgtaatcccagcaccttgggaagctgaggtgggagaatagcttgaggccaggagttcaagccagggcaacatagtgagaccccatctctacaaaagatacaaaaattagccaggcgtggtggtacacctgtagtcccagctgctctggaggctgagatgggaggatcagttgagcttgggaggcagaagttgcagtgagctgagatcatgccactgcactccagcctggacaacagagcaagaccctgtctcaaaaaaggaaacaaaacaacttggacaatggaagggggaaaaagttcctcaagcagccaaaattgcaccaaatggactcccagaagacaagcatttaatttgttaattgagccctctatgggcctgtctgtatttatttaagaaacaatcctatcaagcatagttattgggtttctcagcccaggtagattagaaatagcagattagaggtgggctaggtttctagaggtaaagtacaccagcagaagttagaagtgaaagcaaagagcctaacagaggaagagaaattcttttttttttctttttttagacgcagttttgctcttgttgcccaggctggagtgcaatggcgctatctcggctcactacaacctcagcctcctgggttcaagtgattctcctgcctcagcctcccgagtagctgggattacaggcatgcaccaccacacccggctaattttgtatttttagtagagacagggtttctccatgttggtcatgctggtctcgaactcctgacctcaggtgatccgcccaccttggcctcccaaagtgctgggattacagggataagccactgcgaccggccgacaaattcttaaaactggacacaagaacacaaaacgcttgggctgctgagagattagaacaacaaccctccacagctacacaccttttccacgttatatggcacgttataagtgggtgttcctagtgatggttctgattttttttaaaaaaagtctaaatatgtttaatgttgtctcagaagacaaaatatattttagacagatattcctcagtgatgagtaagcctcagctatctggaaaattcatgcaggcgccagagatcgttactgagtaattcaagctaactgcgtcatgctggttgtaccctgcatgccaatatcagctaaaagcagcaccacgaaagggaaatacgaatctcactaagcactcgcccattcttgttaacgacactggaactgatcatccttaataatacacagataaatctatcaggagcatttccttgcttcctgtgaaaggaagcactcattccatgtgtcctgtgaaattcatccaacttcaggaagctggaggaatacatatggccaagctatctgggcagagagtagacagggaatggaggttgggcacagtggctcacacctgtaatcgcagccatttagaaggcaaaggcgggcagatcacttgagctcaggtgttcaagaccagcctgggcaacatggctaagtcctgtctctgcaaaaaataccaaaaactgagctggatatggtagcacacacctgtggtcccagctacttgggaggctgaggtgggagggttgcttgaccccgggagtttgaggctgcaatgagctgtgattgtgccactgcactccagcctggataacagaatgagactctgtcccaaaaataaaaaataaaatcaaagacacttaaaaagatggggaaaaggaaggacaggcacttaagcaagttataagctactttcctaactacacaagtggaatcttaagctgaggttcccaggagttgactggagccagagaagacagacctataggagcacccaattggagtcaccctccatagtagcccatatgtcttacatggatcagctttcgtggggcccttttactccatctggggaagggcgtcagatctgtggctctcatgtactgctcagtacactgccattcccagttctttttttcaaaaaaaaaaaaaaaatgtctacagaatcggccaggtgtggtggctcatgcctgtaatactagcactttggaaggctgaggtgggtggatcacctgaggtcgggagttcgagaccagcctggccaacatggtgaaactccatctctactaaaaaaaaaaaaaaaaaaaaaattagctggatgtggtggcaggcgcctataatctcagctacttgggaggctgaggcaggataatcgcttgaacctgggaggcagaggctgcagtgagccgagatcacgccattgtactccagcctgggcgatagagtgagactctgtctcaaaataaataaaataaaataaaataaaataaaataaaataggctacagaattaagctggtccaggaatgacagggcttccatttatttgtctttcaattgtgggagaaaaaggatttctgttgagatactgtcgttttgacacacaatatttcgattaatcttgagattaaaaatcctgtgctccaaatcttttaacattaaattatgcatttaaacaggtttgctcctaaatcttaaaatatggaaagcacctcatgaggctaaatattttgatgaccaagttttctggaaggtaagatttttcacctattaacgtgatagattttgagtgcatgaacttaaaaacatacctgagtatatatgttgacttgctgtttatgagtaaaacaaaaacaaaaatggagtaaggagcattgcaggaggaactagaggagaaacaaatccatgatatgcatgtgtgtgggggagggtggcggggaggtggtaaaggtcaccatttccctgatacctcaaattcattcagagtcagggatgagacagctttcactggccacacttcccctccccctatctgcagtcctcagcgtagccaaatagtctgacatgcgggtgacagaaccccacaatgcaaaagctggaagaaacctcaagccttggagtccaaccccttttttgacagatgctaagagtggagacatgacttatcaagatcttacaactggctgggcacggtggctcacgcctgtgatcccagcactttgggaggctgaggtggggcgatcacctgaggccaggagttcgagaccagcctggccaacgtgtcgaaaccccatctctactaaaaatacaaaagttagctgggtgtggtggcacatgcctgtaatcccagttactcaggaggctgaggcaggagaatcacttgaacctgggaagcgaagtttgcagtgatctgagatcatgccactgcactccagcctgggtgacagagcgagactttgcctcaaaaacaaaacaaaacaattgtacatatttaaagtgttgtaaccaagtgagttacagagaaacaccacactttgagcctaattcaggagtcctttattagccggcgacctagagacgactagtgctcaaaattctctcggccccaaagaaggggctagattttcttttataccttggtttagaaaggggagcgggaattgagctgaagcaatcttacagaagtaaaacaggcaaaaaagttaaaaagacaaatggttacaggaaaacaaacagttccaggtgcaggagctttaaagccatcacaaggtgacaggtgcgggggctctgggtgctatctgccggacacaaacgcaggggcactagagtactatcacccgggcaaattcctgggaactgcggacacagcttgccacagtaccttatcagctaattgcactctttgatgtgctgggagtcagcttgcacaagttaagtccttgaggaagggggtgggtaaggagcccttaacgtcttgcaaatgaaggagccgaatggaatccctccggctttcttagctaagagagagtcaatcaagttaatacaagttagggtatcacaaaagtatataatttgatacattttaacgtatttatacactgaagagaccatcaccaccatcaagacaaggagcacacccatcacttccacacacttcctcctgctcctttgaaattcctccctccctacccacctggtcccacccaaaggcaaccactgaactactttctgtcactaaggtttgcattttctgtaatttttttgtttgagacagggtctcactccgccacccacaccgtaatgcagtggcaccatcatggctcactgtagcctcaacctccccaggctcaggagatcctcccccctcagcctcctgagtagctaggaccacaggtgtaggccaccatggcaggctaatttttgtatttttttgtagagatggggtttcaccgtattacctaggctggtctcgaactcatgggttcaagcaatcctcctgccttggcctctcaaagtgctgggattataggcatgagccactgtgcccagccctctgtaatgttacacaaagggaatcatgcagcacgtactgcccttggtctggcttcttttgctcagcatgattattctgagaatcatccgtgttgttgcgtgtaactgacttcatcagcttctctctgcagctgtcagctcttggcttctcccaacagccaatctctctttatcccctgcaagtgttcttgcctatttagcagaatcaaggtactctatcgaaaagactcggaaaattggtttaatctattcattcattcctcaggtatttatcgaataactattctataccaagtactatgctaatcaaccaaggacagcacaaacaggagaaatctccagctcagtcacttgagttgcaataaatatttgctggataggtcaggtgcagtggctcacacttgtaatcccagcactttggggattactgagacgggaggatctcttgagcccaggaggccaaggctgcagagaaccatgatcatgccactgcactccagcctgggtgacagagtgagatcctgtctctgaaaaaaaatatttgctggataaattaaggaaatctgacgaaccccatcagtagccattgcagcaacaggtaaactagaacgagtgtgaatttggaatgaggaaacccgatgttggccatcattctgtaatgtcatgtattatgtaatgtattatatattaatgtatgtattatgtaggcaagttccttgacctctctcactggtaacataagagtagtaatctttgtgctacttcactgggttattttaaagatcaagtgaggtaataatgtctgtaacaacattctgtaaaatgcaaaccgccacatgaatgtgaaagtttattactagggatttagccaaccacaagggaatgtgtgagcataagagctatcatattgcaagcctacagtttctgattttgtgctaggtgcttttccacattacctgattttatcctcacaacagccctgcataaaagtaagtatgtcgcccaggtgcggtggctcatgcctataatcccagcactttgggagcccgaggtgggcaaatcacttgagatcaggagtttgaaaccagcctggtcaacgtggtgcaaccctgtctctactaaaaatacaaaaaaaaattagacaggcgtggtggtggatgcctgtaatcccagctacttgggaagctgaggcaggagaatggcttgagcccgggagatggagattgcagtgagatgagattgcgccactgcactccagcctgggtgacagagcaaggctatgtctcaaaagagaaaaaaaaagtaagtatctcagtcttgaagatgatgaaatggaggcctagagagattaagtaacttgcccaaaatgacagaactaatgcatagaaaagaagaaatgtgatgtcttttggctccaaagacaccccacatatgcgttggttacagttactagagaaaagttattccacccccaccccacccccagaaatcttctgacttgttttctcgcagttgagtaggaccatttattcggcagtgtaccattctcagcttgcagttgaaagccaaatatccattaaagaggcaaggatgcaaacttgctaagctgataaatccaggggtgattttttttttttttgcaaaccatccaacaagacattttaaatactcattgaatttcatagaactgactgccaggattggaaagacattaaagccagctcagccactgcctcgctggttggccagaccacgcctggcacttctgggagggagcactcaccaccccccaagggcacccatctcatcctccgaaggtttatgaaaatgcactcatcatttgctaattcattccactacgtgtattacctaatttgtgacacgatgtgaagtaccagagagataattctaaataaaatatagttatgggtctcaaggagccagatatgctaatctcctatcctcctgcagtttacagtggtcctcaccagatacttatttacaaaaattcagtttattatttatttttttgagacagagtcttgctctatagctcaggctagagtgtaatggtgtgatctcggctcacttcaacctctgcctcccaggttcaagtgattctcctgcctcaacctcccaagtagctgggactacaggcacctgccaccacggctaatttttggagttttagtagagacagggtttcaccacgttggccaggctggcctcgaactcctgacctcaggtgatctgcccacatcagcctcccaaaatgttgggattacaggcgtgagccaccatgcccggccaaaacttcagtttataacacaatctttcacgtgtcttctgctttcattaaaagaatagacagttcccttctttatttcagtttaataaaccatggattttatttcatgctttgcaaaacacaagggctcactgacatgcacttcttaaactaattctggctggtcgcctgtaattccagcactttgggaggctgaggccgacagatcacttcaagtcaggagttcaagaccagcctggccaatatggtgaaaccacgtctctaccaaaaatataaaaaattagccaggtgtggtggtgcgtgactataatcccagctactcaggggcctgaggcagaaaaatcacttgaacccgggaggcggaggttacagtgagctgagatcgcgccactgcactccagcctgggcgacagagtgagactctgtctcaaaaaataaataaatacaaataatgtaaaatacgaaacaagcaatcctggcagtagctgctggaatgagaggagggagaggtcatagggaggtcggggacaatggagcatggagttgtgttggatttggctaagcagcaggaagtgcaaggcattccaagcaagaggaggggggcaggtggggagcatctgcaagaacagaagcagcatgagcaacctggctcggcagtgtgtgaaaaggctgaaaggtggctagagccacttcaatttcatccttcaggcaaatgggaaattcccaaaggtttgagtggggaagcaatgcctacaatgaaagtttgagagtgaagcagagtgatcgaattaagcatgtaggccgagttctgaaataactgcaatgtgctgaagatcatccattggcttctgaatgagtatttgcagtttattttttaaaatgattttattgccaagaaagataaacactactgttttggtacaaaaacataacaaaatgtgttgagtccctcttgctgttttacgcgaagttttaaaaatctactcttgtcacagtggtatcacccctacttctgatttcaaataaatgttctagagacacagtaagggcccaacaaacgcttgttcaacaacacaaggagagccagcttttaaagtaggaaaacaggccgggcgccgtggctcacacctgtaatcccaacactttgggaggctgaggtgggcagatcacttgaggtcaggagttcaagaacagcttggccaacatggtgaaaccctgtctctactaaaaacacaaacattagccaggcgtggtggtgcacaccagtagtcccagctattcaggaggctgaggcaggaaaatggcttgaactggggaggcagtggttgcagtgagccgagatcgtgccactgcactccagcctgggggacagagggagactccatctcaaaataaaacaaaacaaaaccaaatcatacaaaaacattagctgggtgtggtggtgcatacctgtaatcccagctacttgggaagctgaggcagaattacttgaacccctggggggaggttgcagtgagctgagatcttgccactacactccagcctgggcaacagagtgaggagactctgtctcaaaaaatatatatattaaaaaaaagaaaaaaaaaagtaaactaggaaaacacatcagcagcctgccaacagactcccctagcctcggtgagggccagtgttctgggaggcagatctgaattctagtcctagttcacccactggcaggctggtgcccttgggcaggtcgcttctctggggctcagtttcttcctctataaaatgagatcaaatcccatgttctaagagtttgtgctctggagtcagacagatctgggttctaccactgccagctctgtgatcttgtagcttcagtctcgtcatctgacatggagataacagtaactgtctcactgtgttgttagggtttaaaggagataatgtatgtgaaatgttagcaaacaagtgttagctaccctgatttccggtttcagagttctgtggtcccagtttatgccacatgcagtgacgttgtatggtaggctgtggtgtggcaccacttcagaactcagcgcatgcacagcttgcagaagagaaggccagaggagacctaagaaggctcttcgaacacttgaaagaccggcatgtaggccgggcgcagtgactcacgcctgtaatcccagcagttttggaggtcgaggcgggtggatcacctgagtttgggagtttgataccagcctgaccaacaaggtgaaaccccgtctctactaaaaaatacaaacattagctgggcatggtggcgggtgcctgtaatcccagctactccggtggttgaggcagaattgcttgaacccgggaggcagaggttgcagtgagctgagattgcatcactgcactccagcctgagacaagagcgaaactccatctcaaacaaaacaaacaaccaaccaaacaaaaccaaaaaaaaaactggcatgtagaagaaaaatactttttctctacacttctccaaagaatttaactaggcccaggggaggtgcagtataaatttctaacaatctcaactgtctgccaaatggaatgagctacttcatatggcagtagtgagtcctctgtctttggaggcattcaaataaaagccagatggccatttatcaacaatccatgtaaaacgttagatgaaataaaacctatatatccaagatctcttccaattcagattttatgaaagaatttctaaggtctttgtaatgagacatttaggctgtttcaagagatcaagccaaaatcagtatgtgggttcatctgcaataaaaatgtttgttttgcttttacagtttcctcatttggctgttggattttaagcaaaagcatccaagaaaaacaaggcctgttcaaaaacaagacaacttcctctcactgttgcctgcatttgtacgtgagaaacgctcatgacagcaaagtctccaatgttcgcgcaggcactggagtcagagaaaatggagttgaatcctttctctgccactctttgaggagaatctcaccatttattatgcactgtagaatacaacaataaaatacagccatgtaccacataacaacatcttggtaaacaacagactgcatatatgatggtggtcatccagtaagctaaggttaatttattattattccttgtttttttttttttttttttttttttgagatgtagtcttactctgtcacccaggctagagtgcaatggcaccatcttggctcactgcaacctctacctcctgggttcaagcaaatctcctgcctcagcctccaaagtagctgggattacaggcacccaccacatctggctaattttttgtatttttagtaaagatggggtttcaccatgttggccaggctgatctcaaactcctgacctcaagtgatctgcccgcctcggcctcccaaagtgctggaaccacaggcctgagccactgtgcccagccttgtttgcttttttaacagataacagtgtgctcatagaaactgctttgacatgactgcaatcatgtgcttcatagaaacttaattagattataccactagagtcttcagatttttatactttttttttttgaaacggagtctcactctgtcaccaggctggagtgcagtgccgcaatctcggctcactgcaacctccgcctcccaggttcaagcaattctcctgcctcagcctcccgagtagctggaattacaagtgcgcactaccacacccagctaatttttgcatttttacttgacagggtttcaccatgttggctaggatagtttcaccaggatctcttggcctcatgatcagcctgcctcggcctcccaaagtgctgggattacaggtgtgagccaccgtgcccagcctatacttccctttttgaataccatttggtgttttgaagaattaacagctttgtgaacgtggcagtgcttgtgattcaggcttccattgagaccaaggggagaacctggttgcaggacaaacagacggacagcgtgtggcagtgtttaaatgctcttctgaaggctgatacgacagctctctgtgcactgattgcatatgcatcccaagattatattattgttttctactgctatgtgtcacactttgccaaacaggatgtggaaaatgaataagcggttttcttaggcacttcttaacagacaattggtcaaaatgaactccattgcttaagaaacacataaacaccatttagtcactgaacatagctatatgtatggttgttactatgggaaatcttgttttgccaattttctttgaaaattctggcagaccaaggttctttttgtttacataatacttgaaaaataaaaatgaacaagctaacaaactaccaagttttcacttacataaatgtagttgcatacagaaaatgtgactgtgaattaatttttctaggacttttaaactataagcactatttgcacaaaagagaaccaatctatcaattacaaactcacataattttacagattttttttttcctacacagcacataaaacagaaggaatttgaagccaccctccaaacacaggggaaggaggctgtgtgtatatcctcattgtctttcacattctaaggtggttccactcagtgactgaaatccttaagcgttgtattagtctgcttgggctaccataacagcagcttaaactgttgtttagccactcagacttaaacaacagaaatttatttccttatagttctggaggctggaagttcaaggtgccggcaaggttggtttctggtgagacctctctccctgtcttgcagatggctgcctcctccctgtgtcctcatagagcctgtcttctgcttttacacttctggtgtcatcttccttttttttttttttttttttttttttttgagacagagtctcgctctatcgcccaggctggagtgcagtggcccgatggatctcggctcactgcaacctctgcctcccaggttcaagcaattctcctgcctcagcctcccaagtagctgggactacaggtgcccaccatcatgcctggctaatttttgtatttttagtagagacagggtttcaccatattggccaggctggtctcgaactcctgaccttgtcatctgcctgcctcggcctcccaaagtgctaggattacaggcgtgagccaccgcacccggcctcttcctcttcttataaggacaccagtcctattagattagggctccaccctcataacctcatttgaccttaactattatttctttaaagcacctatttccaaatatagtcactttaggggttagggcttcaaaagatgaatctgagggagctcaattcagtaaatagcagtagtcattaatggacaatgtatacaaagataatttcgtgattactgtccttatgcataaacgtcctcagtgttccactgcgtttatccagatttagtatcacaaagactttgctctgagaaaaatgtgatttttttttttttttttttttgagacagagtcttgctctgtcacccaggatggagtgcagtggtgcaatctcggctcactgaaacctccgcctcccaggttcacgccattctcctgcctcaatctcccgagtagctgggactacaggcgtccgccaagatgcccagctaatttttttttttttttttttttttgagacggagtctcgctctgttacccaggctggagtgcagtggcgcgatctcggctcactgcaagctccgcctcccgggttcacgccattctcctgcctcagcctccggagtagctgggactacaggcgcccgccactacgcccggctaacttttttgtatttttagtagagacggggtttcaccatgttagccaggatggtctcaatctcctgacctcgtgatccacctgcctcagcctcccaaagtgctgggattacaggcatgagccaccgcgcccagcagatttttttttttttttttgagatggagtcttgctctgttgcccaacctggagtgcagtgttatgattttggctcactgcaacctctaccatgttcaagcgattctcccacctctgcctcccgtgtagctgggatcacaggcacacgccaccacacctagctactttttgtatttttagtagaaatggggtttcaccatgttggccaggatggtcccgaactcctgacctcaagtgatcctcctgcctcggccttccaaagtgctgggattacaggtgtgagccactgtgcctggccaaaaatgtgatttcttatttcccacattgccaattccatttcaattaactataatagctatgtctattgagcactcaagcgtattctagaaactgttcctgattctggg

SEQ ID NO: 42 - DNA, 65624 bp, Homo sapiens
acccttggcgtggacacatttccagggagggaccggaggacctcctacctcattggtcactgccagtgactgagcttgactcaggtaggagggcatggcaggtattctcagggagtctggtgtttacagaaaagtcatgattacacgtgaaagctgtgggctccctggcttgattcaccacacctgcaggaagcctggctgctcagaccagcacgccgtggacatagcaccacttgctcagcttcatttccgtaactcaggctgccaggcctgctgacaaattttcacgtttgtaataaccctgtgaggagaccagagtacatcttacttgactcataaggaaattgagactgggtgatttagtaacttgggaggcagaattgcaaagtgattagcaacacaagccatggtgtcagatggatctgggttaggtcccacctctgccgtttattagctgtgtggctttgggtactcacgccacctctctgagcagcagtttcctcttttgtaagcgtaatgatgcctacactcacaggcttgagaggaagatccgatgaaatagcatatgcaaaatgattggttccgtgcttggcattccagaaatggtagctgttattcagccaacaaatatttattgagcacctactatggacttccctggtgctgaggatacaacagcaaccacagcagtcaaaagtccctgtctttatgttgctcagattctcataggggaaagcagataatgaacaaatacacggccagacgcagtggctcacgcctgtaatcccagtactttgcgaggccaaggtgggcaagtcacctgaggtcaggagttcgagaccagcctagccaacatggtgaaaccctgtcactactaaaaatacaaaaattagcgcagtgtggtggctcatgcctgtagtcccagctacttgggaggctgaggaaggagaatcgcttgaacctaaaaggcagaagttgcaatgagccaagatcgtgccactgcattccagcctgggtgacagagtactccatctaaaaaaaaaacctaaatacacaagtaaaaatatagacttcgtcagatgctagtaagtgctgtgaaggaaactaaaaggggaacacaaggaacccttgtcaaggggagcagaaaggggagttgatgctgtccttttaaatagggcaatcagaggccaggcacagtggctcacacttataatcccagcactttgggagttcgaggcaggtggatcacttgaggtcaggagttcaagaccagccaggccaatgtggtgaaaccctgtctctactaaaactacaaaaactagccaggtgtggtatcgcgtgcctataatcccagctactcgggaggctgaggcgggagaatcgcttgaacctgggaggcggaggttgcagtgagccgagattgtgccattgcagtccagcctgggcaacaagagcaaaacttcatctaaaaaaaaaacacagcaaaaaagggcagtcagggaaaacttccctgagaaggggatggtggagtacagatccagggaggtgaggtggggagcaagccagtacagttgttccttgactttcgatgaggttatgtcctgataaagccatggtaagtaggaaatattgtaagtcaaaaatgcatttaatacacctaacctacggaacatcatagcttagtgtcacctaccttaaacatgcttagaacgcttacattagcctacggttgggcaaaatcatctaacacaaagcctattttatgataaagtattgaatatctcatgtaatgtactgagtactgtacggaaagtgaaagacggagtggtgggatgggaactctaagcacggcttccactgcatgtgtgttgctttcgcgccatcataaagttgaaaagcgttaagtcaaaccaccgtacgtcggaggccatctgtatctggtaggaggagtgtttcagacagagagaacagcaggtgcaatagagtgcttttttcccagcattttattatgaaaaatttcaaacatctaccaaaaaaagttgaaagacttgtacggtgaaaagccatacatctcacagctagaatcaacaattaacattttactgtatttggtttttgacttatctatcctagatcccttgtgctttctgtagcaggtgacctgccttgaagatttaaagacagaatatcgggaaatgtagtcagaaaatggggccttttataagagtcagaggggaagagcaaaaactctgctttcgagaaatctgtcgggagaggccaactgcagggatacctcccttttttaatgaaagcatttctgttctgcgaggagcgggatcctcttgtcaagcagtcagtccctgctgcttccttactggggcaggatcaggacgcacagggatttggagtgccttggaaccaaccaccacccacgctgtttgccagctggtaaacatgcctgtcaggtctaggggttggcattgcctggaaatctttagtgttcatcttgctgacatctggtgccctcgggtaggtaggtgcagttggctgcctggtttacagagcttgtactgggcccaggttagcaggggtcacatccctttatcccactgtgcaggggagttccttctcaggaaacccagtttataagaagtactgactgccagaaatagagcagagatcagaaccaggaggcaattgtgagaggaatggagacttctgacctctggggattggggtaccctcccccttaattgctgttggggtagcagagggcttagaagcccatgttcctagacttttagaattggaagaagacttagaagtaatctaggctgggggtccccaacccccaggctgtggcccgttaggaacctgaccgcacagcatgaggggtaggccagcgagcactaccgcctgagctccgcctcctgtcagatcagcagcggcattagattctcacaggggcacaaaccctattgggaaccgcgcatgagagggatctaggttgcgtgctccttaggagaatctaactaatgcctgatgatctgaggtggaacagtttcatccccacaccatccctccaacctcaccccggtccatggaaaaattgtcttctacaaaacccgtccctggtgccaaataggttggggacccctgatctaggctacagttaagtggtcaaacacccaggtcctgaagttaggctgcctgggtttaaatcccagctctactgcttactagccctgtgaccttgagcaagtcacttagtttttctgtgcctcagtttactcatttgtaataaaagcttaatagtacccatcccagtgtcatgaactaagttcatatatgtaaagtgcttagaatggtgcctagcaagtacttagtaacagttagctctgaaaatgtataaagcaaaattaaccaatgttttagtggtttgcagccaacttttttctatgcgtgtgctaacatattattttataagagtgggaatatattgtacatgctgttatataacttgctttttcactaaacagtctatcctctgtgtcagttttgataaaagcgttttcctcttgcttttcctgcatatgttcagaaccatcatattggtagcaagtttcatgtcctgcagttttcttaaccaaccccctgctagcggacatttaggttagtctcagttttttccttctgtaaataaagctgcactgagcaagaagtgaccgatgccaagtgactagatgaccttaggtatgacctctctgggtcttggtttcttggtctaaaaacaaaatgacaggattcgactgggtgattaaaatctcctctgatctacataggaattgttttcaagacatttctgcattcctctagtgacagggtgctcactacctcatgagtatttcagtggacaactgtaatggtcaataaagtatccactttccaccttccacttccctgtagctcctggccctggctttattctctggggctccacacattcagtttacactcagtggccagtggctggggccattgtagaaaatgaggaaactccaattccttccttcttttcttcctctttcatcccttcctccctccctacatccctctctctcttccttccttccttgacacttaccatgtaccagaccttctgccaggcacatggatgggagcacagttccgggaagttggctgcagggttagaactaagtcccaagccccgtaaagctcatgccaggggactggactgtccagtactgagggatggggatgctgaggctggtggccttcctcagatgcactgtagtgccccaggcagagtcctgggctgccctgtgaggaggtgaccagaggtagagcaacttcaccctaaggctggatcaggatcccctccaggtttttactagagccaaacccacatctcctttctcttctgccacccccccttaaaatgcttagaaacacatagatttaaatacaagttcaaatgtaagtaatttcaactgtgtaactatgaggagtcaattctacgtgggtcctatctgtatcctccccagggctcagctccattctttgctttcattcattctcattcaatacattgttgttaagagctcactgggtgccctctctgtcatgtagtaaggttttaaaaagaaagcctcttctgagcttcagtttccttatttataaaataggagtattgatccgttccttgcttttcttacaaggatatgctgaagatgactgaagtacagagtaaagaaggattatgtttgggtgtcaaaggaatagaatgccctctttcaaactgagcacagcaggaacctgtaacaggaacacagcaacttgttgaatgaatgacaatattggaaaacatacatttcctcccctccccatcatagtccctctgcttccgtgttaactccatagacaggccagcacagccagccttgcagcctgagataaggcctttggcgggtgtctcccctatcgctccctcaagccctcaagtaggtgttggagagaggggtgatgcctggtgctggtggaacccctgcacagagacggacacaggatgagctctaagtacccgcggtctgtccggcgctgcctgcccctctgggccctaacactggaagcagctctcattctcctcttctatttttttacccactatgacgcttccttagaggatcaaaaggggctcgtggcatcctatcaaggtgagagttcattggaacagtggtcacaggagcaaatagcaggggcaggggcgggggaggcctatggttctccaggggcacagatgttcctttctacaaaatcccgaggaaaagattcccccatcttcttccgtagattgcaccgaaattcagtcaacaatgtaagctttcctttagaagcagcctgggcatgccctcttctgtgaagcctgccttgatttttcagcacagtgagaggcatcctctttggtgttcctcaaattccctctaccaaatggtcttcataattctctgcttctctgcttccccttctctctccttagtggcaaggattttttttatttttatagatttaggggatacaagtgcagctagcttatgcaagcaatttcatgttgttgggttttcgggttttgtttcctttttgtggcctctcgctcatttcttatttctttttgagacagggtctcactctgttgcccaggctgaagtgcagtggcatgatcatggttcactgcagccttgacctcctagtctcaagcaatcttcccacctcagcctcccaagaagctgggaccacaggagggcaccaccatgcctggctaaatttttttttttttttggtagagatgtgggtctccctgtgtttcccagactggtctcaaactcctggacacaagcgatcctccagcctcagtctcccaaagtgctggaattacaggcgtgaagcactgtgcccagctctcttgctcatatctatactagttttcttttggaagcttcagcctgttgctaccccccacccccacccccaccgaccccagctttcttctcacttaggggctgggaagtctgcatgctgtctataaatccagaaccagaaggtatggctgaaggggagggtaggatgatggttattttatattcagctaaaaatattcccagactgtgatgagacaactgtaaataagacagatgtccacaatggtgtgactttgcttttttaaaaatattgaaatgagtttcaggcatctcagtgggctgataggttgttgataatggacagggcctccttgaagaatgtccctgagacaaagttgaagcttgagcctggttgagtgcttgcttgttcctaggttgatatgaacggctagttaactggaagcaaagagaagtcatcctgggggccatggcagtgacaagtaggacttagggagggaagcccttataccatttaaggtgctggcccagagaggagccttcagtgacagacaaacaagagctggcacaattttaattcatttcaatttactttaattcatttcaatccaatacaattcaatgcattccattcattcaaccatgtatgacatccaatgtgggatccagacacatgatgattagaactgatatttatgagcacttactatgtaccaggcactattctacatgctttacattgaaccctcacaataacccaatgaggtgggtactattatgatcttcgtttttcatatgaggaaactaggcatatggatgttgagtaatttgcccaaggtcgctcagctagcaatagcacagcgtatttaaatttagccaccctggatttagtttccttacacttaaccattatgcatcatggccccattttacagtggcgttgagtcatttgtcatataacccagtaggtgtagcagccactattccaaccctgtagattgactctagggtccatgttctttacccctgcaccgtgctactaacgtaggtacaaaatgtcctcagaaactcactttatatggaagctcagaggagggtccacaacccaggcaggggagacgatggtgtcaggggaggcttctggagggaggtgcctgcccagccagctcttgaaggctcagtaggaattacctgtgggacaaaggcgggtcatccaagtgagggcacagtgggtgccattgcgtgtgcacacactagagcagactgagcttgggcttaacattgcattgccctgtagcctaaaaagagaagcaaggggctgggcgaggtagctgacacctgtaatcccagcactttgggaggccaaggctggagaatcacctgaggttaggagttcaagaccagcctggccaacatggcaaaaccccatctctactaaaattataaaaactagccgggtgtggtggcacacgtctgtaatcccagctacttgggaggccattacactccagcctgggcgacagagcaagacttcatctcaaaaaaccaaacaaaaacaacaacaacaacaaaaaacaaagaggagagcagggactgggtgtggtggctcatgcctgtaatcccaaacactttgggaggccaaggcgggcagatcacctgaggtcaggagttcgagaccagcctggcccatatggtgaaaccctgtctctactaaaaatacaaaaattagccggatgtggtggcacgtgcctgtagtcccagctgcttgggaagctgagggaggagaattgcttgaacccaggaggtagaggtagctgagctgagaatacgccactgcactccagcctgggtgacagagtgggactctgtctgaaaaaaataatagtaataaataaaaataaacagggaagcagtgggtggtagactcactgggctgcatacggagtttggcttcagtctgaggtccgaatagtaaacaggagcgcgacaagtttgggtttgggtcatggcggatgccatgccagggctggtgttgggcacaggggaaggggcatggcttgagacacaagaccagcgtggaggctgtagtgtagtattgacccgagggcttcaaccttctgatggtgtacacaccattttttgagcatgtaccatggttatatgttacactttaagtattactacattaatatattttgtatgttataataaatacatacaaattaggaaaattgaaagagatcagaatgaaatatataatattttcaaattactaatcataatggtgtcaatctccaggcagggtccattgctacagttgacgatagtggatgaaaattcactcctcagagtcttcttgataatttgaaattgtcttgattgacttgtcagatctgattagatcgacattttttaaatctcgaatgtgactgacagcttgtacaaggagaagtttcactctgcctttcctttttgttcacttgactgccattatttctctgcttccaatctgtgtttttctgcacgagttggttaagccattacttcattttgtgaaagtttgttgagttaaacttaggtaacttaatctgtcaatccacttaattgaattcagtcctggtaaactataatagattattcaaacctgccaattctaaaaagacattttgagacaatcaggaaatctgaatatagcatgaatatcttacgatatacaaggattattgttaattttgttaggtatgataaaagcatggtgggttttttttttgtttttgttttttaaggctctatctgttagagaggcacattgaaatggcatgatatctggggtttgctttcataccagaaaaaagaaaaagtagagaaggattatagaaacaagattggtctcatgtgacaatcatcagagtttggagatgggcacgtagggtcatcgtgctgttctctctgttttcatatatgctttgaaagttctgtaatagttaattaaaaaaaaaaaaaacaccctggctgagcacttagggaggccaagtggggaggattgcttaaaccaagaagttcaagaccagcctaggaaacatagggagacccccccccgccatctctaaaaaaaaaaaaaaaaaactgtaaaatttaacccagtgtggtggcacatgcctgtagtcccagctactcagtaggctgaggtgagaggcttgcttgagcctgggagcttgaggctgcagtgggacgggattgtaccacttcactccagcatgggcgacagagcaagaccctgtctcaaaaaaaaatgaaaatatttgaggtgaagcgagactgtaataacaaatttaaaaatataaataaaacataaaggctgggtgcggtggctcacgcctgtaatcccagcactttgggaggccaaggcaggcagatcacgaggtctggagatggagaccatcctggctaacatgatgaaaccccatctctactaaaaatacaaaaaattagctgggtatggtggcgggtgcctgtagtcccagctacttgggaggctgaggcaggagaatggcgtgaacccaggaggcggagctttcagtgagctgagattacaccactgcactccagcctgggcaacagggcgagactccatctcaaaaaaaaaatgaaaataaaaataaataaaacataaaaccctgccattagttgcaacatgaagaatatagagaaatgcgtatcaaatccttctcattggaccaatattcccttagggcaccttccaaagctaggagactcaaggctgtatgacatcctgagcaagtgaggggtggcttctgggtgaatctgaatattaaatatttgcagaattgaaaacttcacaaagtacctttagagatagaatagcctagatccatgtttctcaaagtgtggtccccagacctgctgcctcagcatctcctggaaatttagtagaaatgcagattctcaggccctaggccagacctactgatcagaagctctgggcctggggcccagcaatctgtgttttcacaagccctctgggtgattcttctgtgcgtgaaagttcgagaattcctggagctagactgattcaaatcttgcctctgtatcttagagaccttgggcagattagtcaacctctttctgcctctgtttctacttctgtcagaggatgatagtacttgtttcattaagttgttgaaaggataaatgaattgacacacataaagagtattagcttttattatcaaaagctttttttttttgagacagagttttgctcttattgcccaggggagtgcagtggtgcgatcttggctcaccgcaacctccgcctcccaggttcaagtaattctcctgcctcagcctcccgagtagctgggattacaggcatgcgccaccacgcccggctaattttgtatttttagtagagacggggtttctccatgttggtcaggctggtctcgaactcccaacctcaggtgatccacccgcctcggcctcccaaagtgctgggattacaggcgtgagccaccatgcctggcccaaaagctttaatttcttaattttttaaataaaataaataaaactagaattgcttgttttcttccagctaccctggtgattgtattgagcattttctggggtgtgtgttctttgctgtaatgactactggtctggatgacctgtgatgagaccagatgggcaggggcagtggaggagattctagagatatttaggagataaagtcagctgtacttgatgaaaagagtggggagttaagactggctgcagatgtatgatttggcatagagaggtgccagttcctgaggtgagagacagaaggggagggacaggttgtgaggatgaatgaacaatgatatgttcattctgggcttggagttaaggggcctatgatatgcttaggggaagcagagagtatcaattacctattgctgcataacagccaccccaaacttagtggcttaaaatagcaaccttttaatttactcatgatcatgattctgtggtgcaacaactgggctgggttcagctgggcagttcttctgttagtttcacccagggtcattcatgcatctgcagtttggggtgggatggcctcagatgacctcattcacatgtttggcaattggtgattcactgggggccattactgtaacaatcgcctaccaggcagagcttccctaaggctaccaaactgggagactatcctgggtcctgtgctgtggataccactcagtcccccatccccaccccatactcctcaaaggcagagagaggggctactagaagacagaggagttttcccagtgacatgtaaacactccaaaccctggcaccttccacactgcagctttggtctgcccctttgggaaatctctgtttttcttcccaggctgctggaggggtgagagtcgccggtagagtagaggctgtgggcgaggaggtggcggcctcctgaggctgcagtggtctttccaggcagcagtgggagcacagggtggaggtcaaccctagagcctgggggagtgaagctggttctgccttcagagctcttggtgctgaagtttctgcaggccagagggaggggcaagagtgggagggggtgcagatccagaatcacagaggcagctgaccggaggaggcagctgcccaaggggatggactcagaaggccaaagtgctgttatccaaacgaactctttgcaagtggtctctttgcaacaggcctgggggagagcagtcttgcctaaagtcacaccgctaatcagcggccggcacggggtaacagttactaacactcactacgtacccaatgctgggcaaagtgacttgcatgagccagcgagctcaatgctcatggcaatcctctgagcagctggcattgtttcatctcaattttacagctcaggaagctgggacacagaggaagagccaggctctgaacactgacaacctgattgagagacccacactgttcatcaccgttacgctatatatgctgtatagaaaggcaggatggcataatggttaaacctaggtaggtagggtttgaatcctcctgctaccatttactagctctgtgacttggactagttatagcacctctctgtgcctccctttccccctctctaaaatggggataataaatcgtacctcctacctgaggctgttgtgggctaagtctgtaaggcacgtagaacagtgcctggaacgtggggtactgtctatctgtgtgcctgctgttacaacaatggtgagtattgccttatctctcgctgctgaactaccaggttagacttctttctgcaagtcatgaggctttcataaacttttcctgaaggctttccgtagaatgtacaattcccctctgggcccaggcatgggcgcccgggtaggacatccacttcttatcacccctgaacaccttagagcccatcagcttatcaaaccagcagctgatgtgagtgcagagcagactgtgagaggtggaggctgataccagtgaggatgctccaagctgggacccagccctgaagcgggagcccagataatggacgggtggaaatgggcctggagcccaagagaggtgggaggatgagggggcagggggaggagaagcctgaaatcaaatgttatttcctgaccagtttggggtgcatgagctctgtcaacagctcatggaaactgctgccctaatttcatcttgttggctgaggcacaattcctctctcagggacagtgtagagccttggggaggaaggccctgagcgcatatacctggaatcagggaatcgggatcaggggcagcagctgtgcccgataaagcccccacccaggatcctctgacttcctcatctctctttttttttgagccggagtctcactctgtcatccaggctggagtacagtggtgcgatctcggctcactgcaacctcagccttctgggttcaagcgattctcctgcctcagcctcctgagtagctgggattacaggcatgcgccaccatgccaggctaattttgtatttttagtagagacgggatttcaccatgttggccaggctggtctcaaactcctgacttcaagtgatctgcccacctcagcctcccaaagtgctaggattacaggcataagccactgtgcccggcctttttttttttttttttttttttttttaaaaaaagggtctccctctgtcgcctaggctgctggagtatagtgatgtgatcgtggctcactgcagccttaaccttctaggcacaagccatcctcccacctcaccctcctgagtagctgggactacaggcacttgccaccacgcccaagtaattttgtattttttgtagagacaaggtcttgctatgttgcctaggctggtcttgaactcctcagctcaagcaatcctccttccttggcctcccaaagtgctgggattacaggtgtgagccaccacacctggtctgacttcctaatctttagggccccaactctgcccttatccaggcaactctcctctccccatcttccactaacttctttggaatattccagagctgtaaaagccttagagagtatcaagtccaactcctatgtgttacagacagggaaactgaggcctaaagagggtaatggacttgcctaagatcgcttagtgaggtgagagaagaaagagctagagacagcctagcctgtgcaaggacatagttccaggcattcagagctgcgctctgctgccggcatgtttggggcctggtagttagttcactgctgaactaccaggttagattttctttctccaagttgtggggctttcataaacttttcctgaaggtcttccttacaatgtacaattctcctctgggcccggtcatgagcgcccctcacaggctctctctggtccccttctgtaaaatgagaggaaaatggaagaattgctctactcatggaatcttcaataagtctggaccctatgcatatagcattgctacaaaatggcagatgcactttaacaatcgtgtttaataaaaggttggatttgcatatctgaagtggggcatgcagtctccaactgaacacaagcctcactgctcccacatgtgcactgcaccttcatatacatatttcctgcttggctcctgagggaatttgagtaatcccaagaggaacccctgtagaaaatgtcccctggtcacacacccccattcctaaggatgcaagcaggagatagaaacattccctgcacctccctccttgctgtcagaagaagtgcaaagagttgaatccttcctaatgcccacttctcacccacgccccaaatccccaggtcccgtggaggtccttgggggtctcctatatcctggtggtgtcaggttgatttggaaatgtcagtgtcctcccttgtcctctctggcagaccctgggtgtgtgtacgtttcaatggaagtgaatttaaatgtactttataaatcaaagactttttctgagactttggagagttccagtaatgagagcttctcattgttatcaaagccagggctggagaccagtggcaggtgagttcctattgctgtgattgtcatgatgatgttgatgaacagccactatttattgagtgttctccatgtgctaggcactgtactaaacattatttccttcggatgtcccagaaacctctcaggtggctctaattacccttattctgttgataaggaaagtaagcaacttagaagaccacagggctatgaagttgaaacacgtaaattgatattttattttatttatttatttatttatttattttgagacagagtctcactgtgtcgcccaggctggagtgcagtggtgcggtctcagctcactgcaacctccgcctcctgggttcaagcgattctcctgcctcagcctcccgagtagctgggattacaggtgcccgccaccacatccagctaatttttttgtaattttagtagagacggggtttcaccatgttggccaggctagtctcgaactgctgacctcatgatctgcccacctcatcctcctaaattggtatttttatatgtccaaaagagtcaactggtggcaatttagtgaggtttaatctaataggaaatgatagagctgggatcgaacagagctatgtgaactcaaaacctatgcttccccttccaccttttcgaaaaacattgtctaggctgggcacggtggctcatgcctgtaatcccagcactttgggagacggaggtgggtggattacatgaggtcaggagttcgagaccagcttggccaaaaattagccaggcgtggtggtgcgcgcctgtggttcccactgaagcacaggaggctgaagcacaagaatcacttgaacccgggaggcagaggttgcagcaaaccgagatcgcaccactgcactccaacctgggtaacagagagactctgtctcgaaaaaaaaaaaattgtctacatgctggttgcagaaaatttaaacactaaaactaaaaaagtaaaacatctcccaaagttagagacaatattcatgatgggaaaaaaaaaattcttcaagatttctctctctccagtcatttattcatgtgcgaaaacagttggtgattattgataagaagagggagggcagatggtgtggtagtccaaggcacaggctccagcagattatctaggtttaaatcttggctgtaggccaggccctgtggctcatgtctgtaatcccatcactttgggaaaccgaggtgggcagatcacttgaggtcaggagtttgagaccagcttggccaacatagtgaaaccccttctctattaaaaatacaaaaattagccgggcacggtggtgggcacctgtaatcccagctacttgggaggctgatgcaggagaatcacttgaacccaggaggcagaggttgcagtgagccaagatctcgccactgtactccagcctgggtgacaagagtgaaactctatctcaaaattaaaaaaaaaaaatcttagctctacccaccggggcaagttacataacgcctctgtgccttggttttcatatctgtaaaatggtgacagtaacagcacccatgtcaaagtgtggttgtgagaacgaaacaagatagtctatgtaaagtgattaaaacagcgtaggcacatggtaaacgcttaggaaatgtaggctgttataaagctcagagatgttaagtaactagatcaagaccacacagttagagagtgccacagtcttgatttgaacccaaatttgtctcgttctggagctcaagctgctaaccctttttcaaaactggaattaaaccaaagtgctcaccctccgctttgctgggcccctccctgccctcaggtgcatctcttccactcacctgccacagcagcctctgctcagggtctgagactgggaaaggtgagggctacccaggtggccctgatgttttctgccagccagctcaccaggtccctcgcagcaggcggcaaagggagggaggtttgctgtgaagattatgtggttcccaacaacaagagcactgggcctatctctgccctctcttttctgtgtgtcctgggacaagtcacttggcttctgtggctttattttctcatgtgcccagccagggggttggccctcatatgcaataacagcagcaatgacctttactgagtgtccatgtgcatcaagcacgtgtactttacacttgttcttattattaggtttaataatagaataattgccacatttactgagcactcattatgggccaggccctgccctaagtgcttaattagctttagctcctctaatccttaccttatccccacacggcatgttatgttatccccattattcagttgagaacattgaggctcaaagaggcaaagtaacttgaccaaatacttgtaaacgatcttgcatgccccttccagctgccatttagtaagactctaatttcataccaccctaaatctcgtctgcttccccctcctccttctcaccatctccccaccgagcagtcggccaagatctgaccgtgatggcggcccttggcttgggcttcctcacctcaaatttccggagacacagctggagcagtgtggccttcaacctcttcatgctggcgcttggtgtgcagtgggcaatcctgctggacggcttcctgagccagttccctcctgggaaggtggtcatcacactgttcaggtattgggatggtggctggatcacttctgggtcatagagggaatggaccccgaaaggacaggttccagaagatctgggatattgccccctctctgtctagcaccagtgctgtgcaatatttaggacatccttatgctaaaagattattcattgtttaaaattcaaatttaactgggcatcctgtattttactggacagccctactctgtgtatcacaaggaatccaggcctacattcctcctgcatcctttctttcctgttattgtcgattatgattttgtaaagttacataatcagtataagtttatggaaaacgtaagaaggaaacacgttagacagagagaaatagacatgccacacctagagagacattctattttttttttttcttttttgagacggagtttcgcttttgttgcccaggctggagtgcaatggcgctatctcggcacaccacaacctcagccttctgggttcaagcgattctcctgcctcagcctcctgagtagctgggattataggcatgtgccaccacacctggctgattttgtatttttagtagagatagggtttctctgtgttggtcaggctagtctcaaactcctgacctcaggtgaccggcctgcctcggcctcccaaagtgctgggattacaggcatgagccaccgcgtccagcctgagagacattctcttgaaaagaaaggactttcagccccctaaagctactagacaagaaatagccatgcctttattttcattaaattacctgtgctttgtttagatgcctttgtgtgaaatgctaagaaccatcacaactaatgtatggtgccagaagtcagaatagtggttacctgggcaggaggtggatattgattaggaaggaacacaaaatagccccatggggtgcagaaaatgttctctgtgttcacctgggtgatgattacacatcaagctatacacattttaaaagggcattggcacttaatagaaggaactaggctaaattttttcctgaaacattgttttgttttgttcaaacctctgaatctctcagctccccagatgatggtaaacgtcatcctaggcatcttagggacctctcaaggcctctcaaggccattccagcctccccttctaagaccctgctaaacctctgggcactgctgttaaacatttctctatgagccaggaactgtgctgagcactccacaaatattattttgtttaactcttccaggtagggatctaacctggtatacaggtaaggaagtggaagctcagagagggcaaggcacttgcctagggccacacagctaagtggtggagatggctctaacttttttttataaccttttccacatgctccagagtggtcagaacatgaaacacagtctagccagctcctgactggccctagaggaaaaaaactgtatgtatttttcttttttaaaaggtttagaggctgggcatggtggttcacgcctgtaatcccagtacttttgggagctgaggtgggcagatcacttgagcccaggagtttgagaccagcctgagcaacgcagtgagaccctgtctctgcagaaaatagaaaaatcagctaggcgtggtggtgtgcacccacagtcccagctacttgggaggctgaggcaggaggatcacctgaacccagtgaggctgaggctgagtgagccatgatcgtgccactttactccagcctggacaacagagtgagaccctgtctcaaaaaacagttttaggggccgggcgcggtggctcatgcctgtaatcccagcactttgggaggtgggggtgggcagatcatgaggtcaggagatggagaccatcctggctaactcggagaaaccctgtctcgactaaacatacaaaaaattagctgggcgtggtggcgggcgcctgtagtcccagccactcgggaggctgaggcaggagaatggcgtgaaccttggaggcggagtttgcagtgagccgagatcgtgccactgcactctagcctgggcgacagagcgagactctgtctcaaaaaaaaaaaaaccaaaaacaacagttttaggccaggcgcggtggttcatgcctgtaatcctagtactttaggaggcctagacagatggattacctgaggtcaggagttcgagaccgacctgagcaacatggtgaaatcctgtctctactaaaaacacaaaaattagctgggcattgtggcaggcacctgtaatcccagctacttgggaggctgaggcaggcgaatcacttgaacccgggaggcggaggctatagtgagccgagatcgcgccattgcactgtagcctgggcgacagagtgaggctccgtctcaaaaacaaaacaaaacaaaaaccatcttagagttaattcccaccgggattcaatacacacacacacacacacacacacacgcacgcacgcacgcacgcccgcatacacacactgcatccacctggaaagtgacaaagggcaccctggggggaattcaaatggtggtggccctggtttggtgttgctgccttagcttaaggtcacaccagccttcagcctcctgccccacagtctagggctgctcccttcatctgatgtccacagggacctgttcattcttgactcaatccaggaagatgagaagggagagaagtcactcgcagcctgagtgaactcccttgctccacccctgactgcttggatccccctaggggtgacccctgctgaaactggctccttcctgaccggttcccgtcagggctgtgctgatgggtggtgcccaggcctgcccctggggacggggtactctcccttggcaacactccagcttgtgccacttgacttgggactgatttggttctgttttgagtcccttcaggggaggggcctatcttattcaacgttgttgtttgttttcctcacatactgataacttagcaaatggctattggaacaaaaatgaaaataaatggaaccctgaagtgggatgttttaaatttttatttattatttttttagagacagggtcttgctctgttgcccagtctggagtgcagtggtacaatcatagctcactgcagcctctgcctcctgggctcaagtgatcctcccacctcagcctcctgagttaaatttttttacagacgcctgctaccatgcccggctaatttttgtgtttttagtagagacggggtttcaccaggtgggtcaggttggtctcgaactcctgacctcaagtgatccacccgcctaggcctcccaaagtactgggattacaggcgtgagccactgtgcccggcctaaaactgtgtttgagacagggtctcactctgttgtccaggctggagtgaagtggcatgttcatggctcactcagcctcagcctcactgggttcaggtgatcctcctgcctcagcctcctaagtagctgggactatgggtgcacaccaccacgcctagctgatttttctgtcttctgcagagacaggacctcactgtgttgctcaggctggtctcaaactcctgggctcaagtgatctgcccacctcggctccgaaaagtactggaattacagcctcctgagtagctgagaccacaggcacacaccaccacgcctagcttttttttttttttttttgctttttgtagagatggagtctcactatgttgcccaggctggtctcaaactccaggccttaagcaatcctcccacctcagcctcccaaagtgctaagattacaggtgtgagccaccattcctggccttaaaagtgtgatatttttaatgtattttgaaatctgcaggactctccctagaagataatagcaataaccaactcctttattgtgcttgacgtatatcaactcactttgcccttaccgtggctccagaggcattgggtccaccttataaatggaggcaccaaggcacagagtgattaaataagttgcccaggatcacacagccagaaagtgtctgagtcaagattccagcccaggcagcctagacctgagagcacgctcctaaccactgcacatcactgtcttagcacctcctcagcacaaactggcccttgaggaatgaaataccgccgccggcacacacgctcctgagttaagcctttgtcaatgaaatgaacacccacttaaaaggaataacctgtccaggcacgatggaacattgaataaccccttattctaaattcctggtccctgtaagactccttccccatgcccttgcccttttatgaccttcccctaaagtccttgaggcttaagcgggcatagtctgcagcaaacactggggaagctgagtccagacttcagagcacaggctttggatctaggccagctggatttgaacctcacatttgtgatcagctggcatgactgtttccaaaaagtccattttaatcctctacgtgaccctctgtaaaatggggatactgaacggtgagctagcacgattttacagagagtgaatttttttttttttttttttttgtgagacagagtcttactctgtcgcccaggctggagtgcagtggtgcaatctcggctgactgcaacctctgcctcccgggttcaagcgactgccatgcctcagcctcgagagtggctgggattacaagcatgcaccaccatgcccgggtaatttttgtatttttagttgagacagagtttcaccatgttggccaggccactcttgaacccctggcctcaagtgatccacctgccttggcctcccaaagtgctgggagtacaggcatgagccactgcgcccagccttatagggttaaaatttaaaagaggtgatgctgttacaagcctgttttacaaaatgctcttataataaatcattatcatcactgttgctgtggttgtagcatcatcatcattaactcccagagggaggagggagtctcagagcaagctgctcaggggagactggatgtccatggattgtccagctcagtaccacttcctccaggaagtcctccctgataagtccagtcagcatcaccctctccttccaatgaaccccactagccttgtgatatcacagatattcttagttgacaggctcatggtgtatgtagcctgtctagatcataagtacattttttttttttttggatcataagaaccttcaagaccaaaataattttctcctcctgagcatgctcattggtcaagggaaggaaggaatcgtaatagtgttaataaggctagtgtcttttcaggagttggttctttgtgccagtcttggtgctagacacaccgataggaagaatactccttcacatccccaggacaccaacatgggatacgtttgatcatcattcttaatttgcagaaggagaaataggctcagtgagatgaaatagccactccagtggcaaggctgggactggaagccgggcttgtcctgattccaaatccagtttctttccactgccacggagagggagagaagggacagtggccccagatgaggatggggtgactggatgtgggcaggcctgcgggggaagagtgccctctgttgagcatccgaatgatggcagcagaaaagaagactgggcagaatcccagttatcagatcccctgagggaacagtcaccccgatcaccctcagtcagatgagtgtgtgtagatcaatgcctcatagatgaaggcactgaggcacagagtggttaagtcatctgccagaccacatggctcagggtgcagaggccaccttaacgggagaagagatggtcactccactctgcagcatcagcgcccaggtgggtagaaatcttgtcttctatttccacagaaagtaaggtgcccaacagtgtttgttgaatgaatgaatgaatgaatgaatgagtgagaggcatccttccttctcagtcatcctggctctccttctcacccccagtattcggctggccaccatgagtgctatgtcggtgctgatctcagcgggtgctgtcttggggaaggtcaacttggcgcagttggtggtgatggtgctggtggaggtgacagctttaggcaccctgaggatggtcatcagtaatatcttcaacgtgagtcatggtgctgggaggagggacctgggagaaaagggccaaaagctccatttggtggggcttccggggttttgaaaaataaagacaacctgtaatcccagctacttgggaggttgaggagggaagatcacttgaggccaggagtttgagacccgcctgggcatcatagcaagatcctcatctctaaaaagtaattttttctaaattatccagttgtggtggcatgcacctgtagtgtcagttactcaggaggctgaggtgtgagttggaaggattgcttgagcccaggagttagagatgaacctgggcaatatagcaagacctcatctctaaataaataggtaggtggatagatagatagatagatagatagatagatagatagacagacagacagacagacagacagacagacaggctgggtacagtggctcacacctgtaatcccagcactttgggaggccaaggagggcagatcacctgaggtcaggagttcaagaccagcctggtcaacatgggggaacctcatctctactaaaaatacaaaatttagctgcgcatggtggcaggtgcctgtaatcccagctactcaggaggctgaggcaagagaatcgcttgaacccggagggtggaggttgcagtgaactgagatcgcgccattgcactgcagcctgggggacaagagcaagacttcatctccaataaaaaaaaaagaaaaaagaaaagaaaagattgatagatagatagatacccaaatgaggttacaaaagtgtggtctgtgcaaatgtttaaacacaacaaaccagtgcctttaactactacagtataatcctgtaggattgtgctattcatgatgtaattatggttgtataaaagtaattaattctcagagcctcaccagcagtgggtccagcaagtttgtacagccagcatcttctttcagtcagtgcgtgtcagtaactgcacatgtcctctcattgggagagcctgtcgaaagtctaagtttgaaggcagctgtgaaggtaaggccaatccaaatggctctcccagctcctctgctgtaaccctgaccctgagtgaggacatagccaaccttcccatctcataggtgagaaggctgatgcctggagaggggaagggactgcccaagatcacatagcaagatagtggcagaacccaagcgagaacccacagttccagcctggcttagaagaaagtgcactggacttggagtcaaaggctggggtgtgcatcccagctctgccataaatccctgtgtgactctgggcaatttaacctcttagagctttagtttcttcgtctgtaatatgagggtagcagtactaccacatagggttttgagggagtaattgaattaatcacatgaaatgatgcacgtttacaaaaaaaagcatgaagcccctttactgtgcctcagtatcccaaaggactttggatttactctgagaaatacagggagaactagggagtgttgggcagaggagagctatgatctgacttatgttttaagatactctggcttctgggttcagaaaagactgaaggggcaagagaggaagcaggtggagaccagagcagcagtgatggccatcatccagactcagactaggacaatagctgtgagggtggtgggaagtgattggatcctgactatattttaatagcagaattgacaggatttgctgatagactgcacgtggggtgggagagggtcaagatgacttcaaggttctcatctggcacaactcagcagctgctggtgccatttactgagatggggaacattggggtgggatagatctgggagggaaaacccagagttcagtgtcgaatgtggtagcgttagggttaaggttggggcgggtagagatgtgtatgaaacatcccagtggagacactgaatggagatgtacaagtctgaagcttagtggaaaggttagggctagggatataaatttgggagttgttacaatacagatggtgtttaaagccatgagacccaaggagatcactcaggagtgaggataaagagagatgggaagaagtctgaggactgagtcctagaacaccctgcattttagaggggggacatgtgtaagagccagcaaaggagacagaattgtgcttggagaggcaggaggaagcccaggagagcgtgaggtcctggaaggcaaggaaagagagggccccaggtgggctgaatgctgctgagaggtcaagtcggatgagggctgggaagtagccattggatttgacaaggagaccttggcatgcatggttgtagaggaggatgaaggcaaaagcctggcttgactgattcaagagcaggagatgagaaagtggagacagcatgcaggggcagccctgccaaggactttgctctaaaggggaacagagaaatggaggagaagcaggagggcaataatccgatagagaggaaaaatctgatgatacagaagagagatgaactgcaagagtcaagcctttgagttggaaagcaggagtgggattttgagcactgatacctttaggccgatgcagggacagttcatcttttttaaaattattattattatacaacattttatttaaaaatttattttcacagaatacattttcacattagagattcccattgtgcgaaaataacaatttattacttatagttttatatttgtggacagattgttttagaacaagtagaatacatttgagaattaaatctcagtttacaatgggtaatattttgatacgtctatggggaaacttgcccttaaatggaacttctgtatcttcagaagcactccaagcgtttcttcctaggatttagaaatttataatatgagatatcagcatttcctaattttaaaatttccctagtatatgtaaccatcggtaggtggtatctaccgactagagagggaagtttttgaaaattaaacactgtctaattttctgcaaagtttttattcatgaattaagagtatttcccttagtccattattcccaaggcaaatatggaagtttgatcatatgctaatcatactaaagctggattctctttaagagattgagaaattaaaaggcaaaagctgatatatcatgtttagttatactgtgagtcttataagaagctgggaggcaaccccattaactcaccagaatacagaactcagtctcacaacttaaatataattcctctcaaaccttttcctcaaagttaaattctgaaaataatcttgtgattaagagaagaaggctgtccaccaatggacttatctgttatttcttccttattgtgagcttaatggcatgacaaagcagaggcaaagaggcatacatcaattcttcaaagtaggaagtcaaaaaggtcagagcttccacagcatggcaacagctttgcagatgcccacatcgtgatagttgaaatagcaaagcccagcaaaggttaaagctgaaaatgccaaaagccctgccttggcagctttctgcgaggcatccccatgaacatagtcagtaacaacttgtccaaggccccagtgaccatgaagagtgagggctgcagccagggaatagtccgtcgcagagcaaggattcaaataagcagccggaagcagacccgggagcaaaacactgacaaccctctcgctagtccagtggagagatgcagccttggagccagaatggtggctcggtgacaagtgtatgtgctgcactccacaccattctgggataggtcggtcctgaagaaatgctgagatatgagcaggtctgaccactggagttcgcagcaacagagctcggcctccttgggcaccgcaaacggcactcagcctccagagaaccgccatctcgttcctgaggcggagagttcatcttaacgagagaaatggcagggactgtgaataggccggcagatttggtggcgggtgccacaggttcagtctcctgcagggagaggagaaaatgccttactaattccttgtattttctcagagaaacaagaggcaccgtcatcagcctcatgtgagggtgggaaggagggatggggtttgcggagagggaaagtgtggtatggtcatctgtgggagtggaagagagtgagagggctgcaggggtgcagcgggactgcaggctggcaccagggtccctagggcttgtagttggtggaaagtgcatcagtgaccagggctgtgtgcagctgctccaggcaggtgtggaagaagcagagttgaacttgcccagcctggagtgctgcccagagtgagcccaaagcccaagggagaccagagatggggctgtttgcaaaggaggaagtataacagtagcccacaaaatctgagctggttaagaaaggagagagagtgaaaatggggagcccagcctggcagcctgggtacacatctcagctcaacccacactagctgaatccatttgggccccttcgttgacctctctgtgcctcagtttccctatctatagaatggggataagaataaggctacttcctagggctgttgtgaggattgaacaagtgaccgaacacttgttcaattttgaatactgttctaaagcatttaggacagtgcctggcatggggtaagtgttgcggcagtgctgttattttcatcatcaccattgttctcaggctgcgttgattggagctgctgaagggaggcaatttaaggaagtgagccggacagataggaggtggtggtggttatcaggtgcgatgcttgaaactgaggcttcggaggcaacagttactggtaatgacaaggtctaaggcttgacagtgggtggcagaagtgtaacgcagggaaagagacgagcggtcaaggagccgagagggaaggagttgggtggactaagatcatttgtggaagaatgatggagagaaaggctgaagggcaggaactgacatcatcagtgaccaaggggcggccaggaggctgagaccgcagcaagaaagggagagtgtgatggcatcttcttcaagggagctggggatgtttggggtggaaaaaagaacaatggtctgggagggaatatgggaagttttttttttttttttcagatggagtttcgctgttgtcacccaggctggatggcaatgttgcaatctcggctcactgcaacctctgccttccaggttcaagtgattctcctgtctcagcttcccgagtagctgagattacaggcacacaccaccacgcctggcttacttttgtatttttagtagagacggagttttgccatgttggccaggctggtctcaaactcctgacctcaggtgatccacccgccttggcctcccaaagtgctgggattagaggtgtgagccaccgcgcccagcctggaagtttgtatttattaatttttggttgtcttcatctgtgtatgtgactttaacccctaaatacttcagtgtacatttcttttttttttttttttttttttgagacagagtcttgctccatcacccaggctggagtgcagtggtgtgatctcggctcactgcaacctccgcctcctggattcaagcaattcttgtgcctcaccctcccgagtagctgggattaggggcatgccaccatgcccagttaatttttgtatttttagtagagatggagtttcaccatattggccaggctggtcttgagctcctggcctcagttgatccacctgtctcagcctcccaaattgctgagattacaggcgtgggccaccataaccggcctcagtgtatatttctgatgcagttgggttctgtatccccctccaatctcatctcgaattgtaatctccacgtgttgagggcaggaccttgtgggaggtgatgggatcacaggggtggtttcccccatgctgttcttgtgacagtgagtgggttttcaggagagctgatggtttgaaagtgtggcacttcctctctctctttctctctctctctcacctgccaccacgtaagatgtgccttgcttccctttcaccttccaccatgattgtaagtttcctgaggcctctccggccatgccaaactgtgagtcaattcagcctcttttgtttataaattacgcagtctcaggaagtatctttatagcagtgtgaaaacagactaacacaatttcctaaaacaaggggacattctcttacataaccattgttcagttaacaaaaatgagaaattgacattgatatattatgattaccttattctcatttcaccaattttttcaataatatcctttctagaaaaaaatacatattttttgtggtcgaggattacatcttgcatttagttctcatgtcttattaaattccatcaatctggaacagtttcttcatctttctttatctttcatgaccttgacatgttttgaagtttcgagccagttcttttgtagaatgtgggtttgtctgctgttcctcatgattagattgtgggtatgcatttttggtaggaattctccaagagccgtgtgtgcccttcttagtatatcatatcagaagacatgctatcaatttgccccattactgggtgtgttaactgtgatcattgggttaagatggtacctgccaggatcttccactgcaaagttactattttcccctttgtaattaataaacatcttgtgaggagataatttcctatagaaatcctgttgatcatccaactttcacccactgattttagtgttcattgattcttccctgaataaattagtactataataattgccaatggtggttttctaattccatctttccttcaatagttggcattctcctgtaaggaaaagctttcgcttctctgttcatccactcatctatgtatttgtttatattaccatggactcctggattccggtttacacacttccattttctgccttttctctctgcttaatataaggattaatgagaactccctgattcccaggaagaaaatgtcaccagagctttcttaggtggaatgaagagaattcagtgtaagaaccataaaggtgtatctgtgtagtatggacagttttaaaaaacaaacaaacaaaaagaacctccaagggcaggaagtgctgccagactcaggagggcactagaactgactatgagaagccactgagatcccaggtagtctgtgctctccatcttttggctctgattctctctgtacatctaacatctctgtacaccagctttctctttagcgaaaaacgtgtcccctccacccacccatccacctccacttgttcctgcatttctatgtcccagatcctgcagaaaacaactcttttctctcagttagtctcaattctgtagtccagggagagagaatctgatcagtcccctgggtcatttttccactctggtccaagcagctacagctggcatgggaaatagttcacacagtaaaaacatggctgtcaagaagaggagtaaatttcagaggcagaacactccctgtgagcccgaacctcttcctgctttgttgcagtcttcataacgattgctttaaaagactgcattgatataacatcatctctcttctctgcatctttgacttgctagcttaactggtctagaggagggcttagcactgattttcagtattcattttcctcaaaacttcaattcagcctgggtttcttcagcaggagggctcgggggaaccagagccagggaccagagtcatttcagtgcaccagctcaagaaatgaatattccaggccaagaatccccaagtgttctttctgaagtccttcctggtggagctcaaagagatgaaaaacgcaagcccgcttttcagttcttatcaggaaactgcatagactttcctctttatgtatgactgagggctttttaccatcatttgttcacttcacagatatttatttggtatttactatataccaggcactcttgtggcagtggaaaatacaactctcgtggaacatctgttccagaaggaaagactgccaataagcaataaaataggcaaaagatatagcatgttagagagtggtaagtaccacagagaaaaataaaatggagaaaagaaacacgaaaagttggggagagaggacaactgtttgagggggtggccaggggcagcttcatctcatcaagggggtgattttttttgagtacagacctgaaggtaacgagtgcacaagccacatgggtacctgagaacagcggcagaacaatggcagggtgctgggagggctatttaccacccatgctgtttagaattgtcagcacatggtgataaaaaaaaaaataggctgggtgcggtggctcatgcctgtaatcccagcgctttgggaggccaaggcggatggatcacttgaggtcaggagttcgagaccaggctggggaacatggtgaaaccccgtctctactaaaaatacaaaaattagccgggcacagtggtgggcgcctgtaatcccagctacatgggaggctgaagcaggagaatcgcttgaacccagtgggtgaagtttgcagtgagccaagatggcaccactgcactccagcctggcgacagagcgagactccgtctcaaaaataaataaataaataaataaaaataaaaagcagacagactttttagttggctttagaattgttagacaccctctgcagacaaggcaccccgattgcttgcacccagggtggactactccctccatcctgcccttgttacaccctggctgggggtcagcatttcaggcagctgaatgacccaaagtgggaacacgctagtgggtttgaggatgagcaagtggaggagtgcaataggaggtgacgcccgagaggtcaggtgagagtggatcctgcagggtcgtggcaagaacctggaccttgactttgagtgacatgggagccgctggaggcttctgagcagaggagtaacatgatctgacttgcattttattttatttatttatttgacgcagtctcactctgtcgccgaagctggagtgcagtggcgccatctcagctcactacagcctctgcctcccaggttccagtgaatctcctgcctcagcctcccaggtagatgggattacaagcaagcatcaccacgcctggctaatttttgtatttttagtagagacagggttttgccatgttggccaggctggtatcgaactcctgacctcaggtgatccacccacctcagcctcccaaagtgctgagattacaggcttgagccaccacgcccggcctgacttgcattttaacagggtcactctgtctgctgtgtggagaacagtccgcaggaagacaagggtggaaatggggagaccagttaggaggttactgtaacaatttggggtagcggtgatggtggcttaaaccaagatggggtcagtgggaaatggtgctaaaaatcctgccaattctgggtatttttagaaagcacagctgacagctttctccagtagcccactaaataagttatgaagcattactaaaatgtgatagtcatgatgcaaaattagaatatatctagaatctcccgaagaccttagtttggtattacaagaagtctggttgcttcatgttgcaaaatttatatcactcatcactcctgcagagttaaaattccgctgagaagtaggaatcagtgaagtgcgtgtccatgtgggtttttgccacacctaagtgaaccttggtcaaaagcatataagagctactgataggccgggcgtggtggctcatgcctgtaatctcagcactttgggagggaaggatctcttgagcccaggagttcgagaccagcctgagcaacatagtgagattccatctttacacaaaatttaaaaattggccaggcatggttgtgcactcctgtaatcccagctacttaggaggctgaggtgggaggattgcttgagcctgggagttggagactacagtgagctgtggccacaccactgcactccagcttgagcaatggagcaagactctgtctcaaaaaaaaaaaaaaaaaaaaaaaaaagaggccgggcacagtggctcatgcctgtaatcccagcactttgggaggccgaggcgggtggatcgcctgaggtcaggagtttgagaccagcctggcaaacacggtgaaaccccatctctactaaaaatacaaaattagcccagcgtagtggcgcatgcctgtaatcccagctactagggaagctgaggcaggagaatcgcgtgaacctgggaggcaaatgttccagtgagccgagatcgtgccattgcactccagcctgggcaaagcctgctgggttgggctgggtaagctctgaacaccagtctcgtggcttcaagtcacacctcctaagtgaagctctgaactttctccaaggaccatcagggctttcccctgggcagaggatgccgacactcactgctcttactgggttttattgcagacagactaccacatgaacctgaggcacttctacgtgttcgcagcctattttgggctgactgtggcctggtgcctgccaaagcctctacccaagggaacggaggataatgatcagagagcaacgatacccagtttgtctgccatgctgggtaaggacaaggtggggtgagtggtctcatacttgggctgagcagaatggctcagaaaaggctctggctgaaaaaatctccctcctttaccaacttcccctgggtgtctgaagcccttccatcatgattcacttctttgagtagtgtttgctaaattcatacctttgaattaagcacttccttttagggacctctcttcattaatatccactagaaaggagagactcattatgtgtgagtttcaataagtttatccaatccctttgttttcaactgaaaggagggaaacggacaagtgaagaaggtagggcccaggagtgaaggaacaagggtgggaatagtaataatgttgtactttgaaaatctactgggaaaatgatgaacttagactgctgggagaggctaatagaaaatcgggcagtgagcttgatagtaggcaaaggactatcaggccacggggtcaagttaaagcagcacattcattaaaaaaaaaaaaataagcgtttgggccaggcgtggtggctcaagcctgtaatcccagcactttgggaggccaaggtgggtggatcacctgaggtcaggagttcgagaccagcctggccaacagggcgaaaccccatctctactaaaaatacaaacaaatcagctgggcatggtggtgcacgcctgtaatcccagctacttgggaggctgaggcaggagaatcttttgaatccaggtggtggaggttgcagtgagccaagatcgcgccactgcactccagcctgggcaacagagcaagagtccatctcaattaaaaagaaaaaaaaattaaaataagcatttgaccatcacagagcaggttcaggaggcctggggtatgcagatttcaaccctcttggcctttgtttccttgtctgtaaaatgtggttagctggtatcagcttgagagctcggaggggagacgtgacttccccatctaactctaagtgacaaggctgagactctccagccctaggattctcatccaaaacccctcgaggctcagacctttggagcaggagtgtgattctggccaaccaccctctctggcccccaggcgccctcttcttgtggatgttctggccaagtgtcaactctgctctgctgagaagtccaatccaaaggaagaatgccatgttcaacacctactatgctctagcagtcagtgtggtgacagccatctcagggtcatccttggctcacccccaaaggaagatcagcatggtgagcagggcgctgcccttgggcagcacttgggtctaacaggactagcacacatatttatgcccctccccaccccagggccagcgtgggttgggagagggcatgccgggtggtggagctgtgcctgcctctacagtggagctctaggaagaatgctgggtggtcacagggggcctgggactcaggagactgtccagtgatcaaaggctttctggggggagtgattaaatccatccatgctaacatgaaacagacctgagtttgaaccccgtttctgctagttgctcaagtcagtcaccatgagcgagagtcagcagcaacagactagactagaattagccagcctctctcttccccccaacaaatttcaagaatggaaccatcagaatcagaagtagagaagtatgtgacactagccatgtggctctggtcaagccacttcaacgttttgagtctcagtggcctcatctgtaaagtgagaattaagagatggtgcatgtaaagtgcttaacggggagtaaatggtaggcaaacattagctgctgctattagtacagagagacaatggtgtgtgtgagtcttgtgggcagagatgggtgagaggggagacaaaacaagttctcatgatgatgggggcagggggtccagctggtggtgtcggagggaagtctggacagaccagtggtggggctcgggtgggaggcactgggggggctggagtggaaagaatgtggccacagatgacagcttcacagcagaattcagtgctaagaggaagtgagtggccatgagttccatggtgacagaaagtctaagacacctagcaaggcaggagtgggtgtcagctcagggaagctcagaggctaaacctaggtgagagctgagggtgtcagataagagcaaggcaaggctccggttctggagtagtgaaggacatagcagagctataacccaggaacaaggcccagcttattggaactgggaccagtcacacagggtggcacaggcaccaagtagccaataataataataaaaacaataacaatgatttatgtctattgggcatttattcatgttctatgccagacactggactaagagctttatatgtggaaactcatttaatccttacaataaccttatgaagaaggtacatccaaaaccccattcttctaggccaggtgcagtggctcacacctgtaatcccaatattttggaaagctgaggcaagaggattggttgaggccaggagttcaagaccagcccaggcaacatagcaagaccctgtctctaaaaaataaaacaaaaacccattcttcccgctgtccagggacacaccactaatgagtgtgatgggtgcctaggatgctgagcacctggacttcccagctcattccctaaatgctgcacaatcagggtaactgtgccctgagcctaagaggcagtagtgagctggcccaccgtgtccactgatgaaggacacgtagccccaacacaggggagaggtggtttcaggatcagcaaagcagggaggatgttacagggttgccttgttcccagcgtgctggtcacttgcagcaagatggtgttctctctctaccttgcttcctttacccacacgctatttctttgcagacttatgtgcacagtgcggtgttggcaggaggcgtggctgtgggtacctcgtgtcacctgatcccttctccgtggcttgccatggtgctgggtcttgtggctgggctgatctccatcgggggagccaagtgcctgccggtaagaaactagacaactaatgctctctgctttggctgaaggccagcaggacgctgggacctgatgggccactgtgcagtgcacagctgcattaggcaggtgttggtgcattctcttattggcttcaacgcctagcgagggatccatcctggctcggtggcacatttgttaagatgctggggagcaggtggcagaacccatttgagcttgcttgggcactggggagaatttgttaccaggctacaggggtgtcacagaactcaaggacagggactggagtgttgtggggagcccagaagcccctgttttacttctttctttgcttttcctgaatatctgctttattcttactctatagacctgcttcctcctctttcaccccacattgtggggtgtagtcttttgcttcaagaaagcagcctggtggatggaatctcttggccccaatcccaaattctctggagaaggggctctttggtttaacttggataatgttgtcttcagctgggggtgggcacatcgtgcatatgtggctgctgccggggaaccacgtggatgatgtgagaggagcagcacccagaagagggagtgctgggctgatggtccaggtcgtgtccacttctgattgtttaattcttcttctaagtggatggatctttctccaatactcagcaaatcctgatcgttccagaatacttcattatagccaattggttataatgtgcttctctaagagaaatatttagggacaacaaatcttcatgggtttgaagacttgatggaggaaaaaggagtagattttcgaaggctggatttggatgaacaggggctattcagggagtgcattccaacctaaaattaggaaaaactggctgggcgcagtggctcacgcgctttgggaggccgaggcgggcagatggcctgaggtcaggagttcaagaccagcctggccaacatggtgaaaccatctctactaaaagtacaaaaattagccaggcgtggtggcgggcacctgtcatcttagctactcaggaggctgagatgcgagaatcacttgaacctgggagacagagcttgcagtgagccgaaattgcgccactgcactccagcctgggcgacagaacaagactctgtcttaaaaaaaaaaaagtgttttatatacagagtggaatattatttagccataaaaagaatgaaatcctgtcatttgcagcaacatggatggaactggaggtcattaaaaaataaaataaaataaataaggaaaaacgtatcaatacttcgattgaccaaaaccagggcaaatctgattttcatctttgcaaggggaacaaatttcttttatctcctctggctttgaaaccctgaaatgaaaggaggaagggcagaaaaaagaacacatagcaagttaccatcaggctcagcgcccatcgcattccctgagcttgtttccttgacttcatcactggcaggactattcaaaaatgattccctcattcattcatatattcattcattcatcattccttcattcaacacatacgttttaacactcatcttgcttttcaagctatagtttagtgagcgaaatggatacacagaatacagtgtgagaacagctacagggcacatctgagctagcctgggatgggtccggaaatgcttcctggagcagaggaaacggttgacagccaagtgttgacagagaagtagtattagccaggcagagacatggggaatgtattccaggcagaaggcacagtgtgtatgaaagcttattggtaagaagagtgtgtggcccaaccaggaaacagacattctgaaggcatagggtccacccaggagcatggtgaacccagatccctgaaagatgggaggtgctcaggcacacttcctgggctagttgaggggtctggatttttatttacttatttttttatttattgagacagagtctcgttctgtcacccaggctggagtgcagtggtgcaatctcagctcactgcaacctccacctcctgggttcaagtgattctcctacctcagcctcctgagtagctgggattacaggtgcccaccaccatgcctggctaatttgtgtgtgtgtgtgtgtgtgtgtgtgtgtgtgtgtgttttgttgttgttgttgttgttgagacggtgtctcgctcttttgcccaggctggagtgcagtggcgccatcttggcttactgcaagctctgcctcccgggttcacaccattctcctgcctcagcctcctgagtagctgggactacaggcgccctccaccacgcccagctaattttttgtgtttttagtagagacggggtttcgccatgttggccatgctggtcttgaactcttgacttcaggtgatccacccacgttggcctcccaaagtgctgggattacaggcatgagccaccgtgcccgacctggatttttattctgaagactaatggggatcctaaggaaggaaccagcctgactgaatttgcatatgtgtccacatctgctggctcatggctgtgtgggaggctgagtgatggggaggaaggattactgagtagggatctagaggtgtggcctcatgctttctttctaaccagctgtgttgtctttgggatggtgcttaaatttgggctagaccagtgggtcttggtcaccccccaggggacatctgacaatgtctggaggcgttcttggttgacacagtggggtgagggctgctactggcagctcgtggggagagaccaggaatgatgcttaacatcctacagtgcacagggcagcccccatcacaaggaattatcagctgaaattgtgaatagtgcctacactagacccttgctactcatagtgtggtccgtagatgagcagcattggcatcacctgggaccttgttagaaatgctcttagaccccaccccacatccactaaagccagctcttcatttcaacaaactccccattgatgtgagtacacattcaagtctgagaagggcttctttgaggtgagccttagtgcccatccccatttggtggcgccggataccaagggtgtgtgaaaggggtgggtagggaatatgggtctcacctgccaatctgcttataataacacttgtccacaggtgtgttgtaaccgagtgctggggattcaccacatctccgtcatgcactccatcttcagcttgctgggtctgcttggagagatcacctacattgtgctgctggtgcttcatactgtctggaacggcaatggcatgtgggtcactgggcttaccccccatccccttaacactcccctccaactcaggaagaaatgtgtgcagagtccttagctggggcgtgtgcactcggggccaggtgctcagtaggcttcggtgaatatttgttggctgatttattcagaaattatgtccagcccctaccttggatggatttatcacctctccaggccacctcttctttccaaataggaccacctaggtatagaccaaagacacgaaatcttctgtgaccccacaaacacagagcaggtcaaataggcccaagccaattgagactgtggttcaggtcgtgatgcagagctttgctgtggacgtgctcccactgcgtactagctgggcatgcggcttaacctttctcagcctcagtcgcccccttgtaaatggagataaggatactatctcccctcacagggctgttgggatgctactggatttaataagctaatgcagggacatgctaagcacaacccatccctgaggcccagagaagggtgggcctcggctgaggtctcactgtgaggtgggaatgtgggcctccagaccagaggtaggtcctgtggcccctagacagtggacagcaatggtcagtttgacacaccagagccctagccattacttcctggatgttgtgtgaatattttctggacatggcttatataaaatgaaaaagtgaattgggcacgatatagggatagatttttagagatgaactgatagcatgatgataatcatattcactgataacatttactactgttattgactgctttaaaagtgttgggcattgtgctagaaaccattatatgcattatctccttgaattctcacaaccgcctactgaggtattctcagactctaagaaatgagatttaagagaagttatctgcccaaggtcacccggctggaacctggctgtaaaaatggctgaagcaggtgatgaggagctgatgtgtttggacgtgtctcagagaaatcatggaggcgctggggttccttccggttcttggatgccttctacagagacaaccatagccccaaattatagggatcacatatcagtgggtgagacatccttgcttgggatgaggaggggatgagctgtgtgaagcaaggtgcctctgtaatgggttccagtgatgtgtctgccactgtcttaataactgtgcaattctaagcagaacctttcctgtctctgggcctgagagttcccctctgtaagatgaggacttgacctagcaaggtcctactcagatgcctgtagagaacaggcaggggaagttagaaaaaaaaaaagccagtgaaggaagggagctcttcagcttgcacccaccatcacagtgcagggacccaggctcagtgttgccagatccaatgacttctcaagagctcaaaatctagagttttgcatgtgctctcccaagtactggcagaaaattcaagattgttagtaacactgtgtggctaaattctgcttgtgggctgcctagattcccaattctgtgattctgtggttctctggaagcattggttctccacagcacctgcatcacttggaaacttgttagaaatgcaagccctacctacggccccaccccagacctacccagttagaaatctgggggtgggacctatcagtccatgtttgaacaagccccacaagtgttctcttgcaagctcaagttttagaaccactgacctatagccaaaaaagaaaaagccaatcagtggtttgctggtagaggattaacttaacaactggctttccatgaaaataaagccttgattggtagcacttgcaatttctatggtacaaacgcttcccacatgactgagttcaagctatcaaggagacgtcactgcacatggacttgggaagagatgagaacaatcagcccactgagcctatgggaactggctccagcacatccctgcaagtcaactctcatcagggtgagtgagttgaggaccaagaagcagttatcctcttgcctttgcaggacccaggcaaagggaagggcatagtgacagtgatgatctctcttccggaagtctttggtttgctgagagtaaaaggcgtgggcttcaccagtggtgaagccagtcatgcagccttagtcctggtactcaaactccctaaatctcagttttctatctgtaaaatgggaaaataagtcctatgtcacagggttgctgtgcagatttagcaatagaacatagccccgttctttatgatgactgatgctgcatcagtatggggacatctctatgtaatggaaagatggagagaggattaagtgcaaagtcacagcacttaatgggaactgtggattagctacttggtggcattgggcaagtcagttgactttgcattaattccacaaacaatatttcccaatttcctattcagatgagcatatgtgactgagtcagatgctgtgatcagagccaggatggagcatttcccacaaactgtgggatttttaagtgatgggaaggcacactgaaatggcattgaatcatgcagttgcagatactctttttcaattctcagtcctttgattacatcagggagaaaagaaagtccccacttgggctgagaatctctgcacccttctagctcttgttaaccactcttttgaatagcagagaaaacctcagactgccatatctgggagagattttagcaacattttgttttcattgtatctctttttacagctacctcccatttcccttctatttcaagctagtaacacagttttcttttaaattcatttatttaaatgtaaaaataagtctatttggagaaaaaaaatttttaatagcatctctggaatgccagtatggctaaattcatgaatgttgtcctcaaatgctgaaatctgggaagcatctggccaagctttgtggacaggccttcctagtttgaatcccaagagccactcattccgagccacaaaacattggaattcttggttcacttccctaacctgaacttgtcctctgtgaaatagggacattaatagctcactcacagggctgctgtgaggacatgtgttgagctgagggtctggccaggggagaccctgtgcagggagactgttatcatggtgatggatttctgcttcattcatttctttttccagacagcatcatatagaatgagttgtggggtggcagtcagcaggtttgggtttatcctctattctgccacttattacttaaaaaaaaaaacccaacttatatagtataagctatatccagaaaagtgcaaatatcatacaagtaccatttgatgaatcttctgatatccccacataaccaacacccagaacctcttcttgtctcattccaggataaccactaacctgacttctaacagcatcagtcagttttgtctgtttttgtacattatatatgtgatggtttgaatgtgtcccccaaatttcatgtgctagaaacttaatccttcaattcatatgttgatgctatttggaggaagggcctttgggaagtaattaggattagataaggtcatggggtgaggtatgatggcactggtgacttataagaagagaaagagaaatctgagctggcatgctcttgccctctcaccgtgtgatgacttctccatgtcatgatgcagcaagaaggccctcaccagatggtggcaccatgcttttggacttcccagcctctagaactgtgagctaaatcaatttattttctttataatcacccagtttgatattttgtcatagcaacagaatatggacaaagaaagaaaattaatgcaagaagtagagtttttactgtaacagattcctgaaaatgtggaagtggctttggaactgggtgatgggaataggttggaagagttttgaggagcaggctagaaaaagcctgtattgtcaagaatggagcattaggccaggcacggtggctcagacttataatcccagcactttgggaggccaaagcaggtggatcacctgaggtcaggagttcgagaccagcctggctaacatggtgaaacgctgtttctaccaaaaatacaaaaaattagctgggcactctggcgcacacctgtaatcccagctactcaggaggctgaagcaggagaatcacttgaacccaggaggcagaggttgcagtgagctgagatcgtgctattgcactccagcttgggcaacaagagcaaaactccaactcaaaaaaaaaaaaaaaagaaaaagaaaaagaatggagcattaaagacagttctacagttctggtgagggcttaaaagaagaccccagaactagggaaagtctggaacttcttaatggttactgaagtcgttgagatcagaatgctgatagaaatgtggctggtgaaggccattctgatgaggtctcagatggaactgaagaaccacgtgttggaaactggagcaaaggtcatcctttttataaagaagcaaagatcttagctgaactttgtctgtgccagagtcatttatggaaagcagaaaatccgtaggtcacccatgttgtagagaatgaaagaacattttcagctgagaaaactgagagtgtgaccaagctaccgattgataagaaaactagtacacataaattagccaggcgtggtggtgggcgcctgtagtcccagctacatgggaggctgaggcaggagaatggcatgaacccgggaggcagagcttgcagtgagccgagatcgcgccactgcactccagcctgggcgacaaagcgagactccatctcaaaaaaaaaaaaaaaaaaaaggaagaaagaaaattagtacacatagaacaaagccagaggctgttcatcaggacaagggagaaaaactccaaagccatttcagagatcttcaagactgcccctcccattactggcccagagctctaagagggcagaatggtttggaatgaccagctgctgcccagggctgccttgggtctctgctccccacatttctggtgcagcattcctcagccatcccagctgtggttcaagtggccacaggtgtgatgtggaaggtaaaagtcataaaccttggcagcatacacatggcactaattttgcaggtgtgcagaatgcaaaagctgagggggcatgccttctcccacctacatttcaaagggtgctgtgaacggccaccccagagagcccctagtagagcaaggtctagtggagctacaagggtggggccaccgccaagaccccagaatggtagagctatcatagtgcaatgccagcttgggagaactgcaggcatgagactccaacctgtgcgaagtgcaacatgggcagaacccagcaaaaccacaggggcagagctccccgaagcttcgggggtccaaattccatagtgtgtccaggaggtggcacacagagtaaaagatcattctgaaggtttaaggtttaatgttgttttctatgttgggttttgtactttcctggaaccagttaccctttttcccttgcctctttttccttttagaatgggaatgtctgtcctatgcctgttccactgttgtattttggaagtcaataacttgttttgactttacaggcttacagccagagggaatctcccatagaatgaattgtaccttaagtctcacccacatctgatttagatgagaccatggactttggaattttgagttggtgctggaacaagttaagactttgggggttgtctaagtgtggtgtttcatgcctgtaatcccagtgatttgggaggctgaggtgggaggattgcttgagcccaggagttcaagaccagcctaggcaacatagtgagacctgtctctacaaaaataaaaataaaaagttagccaggtattgtggcatgtgcctgtaattctagctactcaggaggctgaggtgagaggatcacttgagcccaggagtttgaggctgcagtgagctatggtcgtgccactgcattccagccagggcaacagagtgagactctgtctctacaaataagattaaataaacgtagctggagatggtggcacacgtctgtagtcctagctactcaggaggctgagacaggaggattacttgagccaaggagtttgaggctgcagtgagctatgatcatgccactgcattccagcctggatgatagagcaaaatcccatctttaaaaaaaaaaaaaaaaaaaaaaaaaatatatatatatatatatatatatatatatatatatatatatatactttggtgctattgggatgaattttgcatgtacgaaggacatgcattttgggggctggggcagaatgctatggtttgaatgcatccctcaaatttcatgtgttggagacttaatctccaaattcatatgttgatgaaattggaggtgaagcctttgggaggtaactaggattagataaagtcatcagggtggggcccctatgatgagactggtggcttacaagaggaagagagacctgagctgacatgctcttgccctcttgccatgtgataccctctgccatgttatggcacagcaagaaggtcctcaacagatgccagcagcatgctcttagacttcccagcctccagaaccatgagctatatataattattttataaattacccattctgtggtattctgttatagcaacagaaagtgaactgagataatatacatggaatcatacagtaagtctgtgcttttgtatgcttcttttactcaacattgtagttgtgagattcatccaggttgttaagcattgctgtactttttttccactgggatatagtgttctgtcatgcttgggtcttaatttataaaggtgactgagtggcattttcttccagtattattggaaggaaagttttgttgttcacagttcccctgtaaaaaagaggcagaacacgtcttgcagggccacacaaaactgtgtcatccagggaccaggcagcagaaagagagggggaactgggcctatgcctttatgaaaaagagtggtgggagagtaactgggtgagggcatccactaatgggcaggaagtgaaaacacatatgttggaatttgtagctgaggggtttataatatgagtttcccatgcctgagaaagctgacttgcaagaaaacgagataaacaactttggccattagtgtggccctgtcataaatgaatgccggatagacaaatcgagaatctaagaaaagatagttggaacaagtgttccattgtgtgaatgcagcagaatttatttatccattattgaggaggatttgggtagtttccagtttggagctattatgaatattctagtattgctcctctgaacattctagcacttttgtttttggagcacacgaatgcacttctgttgattatatgcctagaagtgaaattgttgagttatacagtattcacacagtcagccttagtggctactgctaaacagttttctctagtagtttgcgccaatctaatcaccagtagtgtatagaagctccttttactccacattttgttaacacttggtgttttccttctttttgattagtcatttagcagtgaaacctattttttacattttgatatctccaataactaactaaatggagcacttttaatatgctttttggacagttgaatatcttttcttgtgaaatgtctattcaagttagtttgcccattttctattgtggtgttctgtctttttcttattgattttaggaattccttacatatcctggatatgaatcccactatgtggcttacctttttccttctttctttttgaaacagagtctccttctgtcacccaggctggaatgcagtggcgctatctcagctcactacaacctctgcctcccaggttcaagcaattctcatacttcagcctcctgagtagcttagattacaggtgcatgccaccatgcccaccgaatttttgtatagacaaaataatttttggtagagacagggttttgccatgttggccaggctgatcttgaatcctagcctcaactttggcccaccttggcctcccaaagtgccaggattacaggtgtgagccaccatgcccagcccaccttttactttcttaatggtgtcttttgaacaaggaggttcttaattttaatatagcccaatttatcattgttccctttatgcttagttcttttatgtcctgtttaagaatttttgcagccagctcggtggctcacacctgtaatcccagcactttgggaggctgaggctggcagatcacaaggtcaagagatcgagatcatcctggccaacatggtgaaaccctgtccttactaaaaatacaaaaaattagctgggcgttgtggctcttgcctgtagtctcagctactcgggaggctgagatcacgccactgcactccagcctggtgacacagcaagactccatctaaaaaaaaaagaaatttgcaaggtcatgcatatgtccccctgaatttttttctaaaaatcacttaattttagatcaatgaattgagtaattgactccatttttcagtcattcaacaaacatttccctgaggttttgataacctgaactgtgtttggagctggggaggaagcaaactattgaatatatacaaagatggcaaagatgagggcctggagcttgccacacggaaggggggatggctgcctgaatggttgggcaggtagttgttgacatctgcactccctacaagagcagcagggtggcaactctttttatctttttaatttatttttcttttctcttttttttttgagatggagtcttgctctgttgcccaggctggagtgcagtggcgtgatctcagctcactgcaaactccacctcctgggttcacaccgttctcctgcctcagcctcctgagtagctgggactgcaggcacctgccaccactcccggctaatgttttgtatttttagtagagaaggggtttcactgtgttagccaggatggtctccatctcctgacctcatgatccacccgcctcggcctcccaaagtgcggggattacaggtgtgagccaccacacccggccttaatttatttttctagtctgcaggtaattctttttaattctctccactctcctatgatcttatgaggtagggactgtgattatttctcccactttataatgaacaatcagtaaagacagggaagataaccaaatgacatacaaggtggggtccaccccatgaggctgcaggcttggagctttcctttgtcttaaaaatgagaacatgagctgcccacctgttgagacaagaaataggaaaggcttaaaaaactggcttgttgtgtacaactatccgtggggctgcagtgaacgggctggcagtgcccaggtgcatgctgaaccctgggacaatcacattcagcatccaggggcccccgtaatagcttaatgtttgaattgaacccctggggttgccttgaaggagagagatcctggaagtatgttcaaggggtagggatgggcaggggagatgggtctgaaagccaagctctaccccacccaccttgccccaagagaaatagaaccttcatctttaattgcctaacgagaaaactggggctggccagatgtggtggctcatgtctgtaatcccagcaatttgggaggccaaggcgggcagatcacttgaggtcaggagttcgagttcagcctggtcaacatggtgaaaccccgtctctattaataatacaaaaattatccaggtatggtggcgcatgcctgtagtcccagctacttgaggcacaagaatcgcttgaacctgggggacagaggttgcagtgagccgaccactgcactccagtctggacgacagagtgagactccatctcacaaacaaaaacagaaaaaaaaaaaaaaaaaagagagagagagaaaactggaggctctgagaggttaaaggacttgcccagggtcttgcagctagtaagtgacagagctgggacttgagcttgggttttctgactcctggtctggttcattatccatgaggtgctgggaactaaaataagccacaatcttggaatctccgtcgcctccctccctcccacatgtctgcgtggctttttgggaaaatgccaggggaatgtaccagccagggagaggacccttgttttcctcatggcccttcctggcaatggcactactgacaccgacagtcctttttgtccctgatgacctctgctgcctgatgcccaagtgaccacctctgctttgtcatttctaggattggcttccaggtcctcctcagcattggggaactcagcttggccatcgtgatagctctcacgtctggtctcctgacaggtcagtgtgaggccacctttcttccaccattgccaggacacagcacccacgtccagagcgcaccctgccgtgtggctggatgtctatgtgccccatctccttccctgaggatcacataatttcagaattggaaaggttcttagaggtcacctgctgctaatgtggactgtgaggccagggcagggaagggacatccctgaggttataagtagggtgagtggcaacgttgcagacttttgaacccagggctggtgatcacactcagttttgcacagaagcccgagaaaatccttacacccaaaagcctaccttttatttctgaggacacccataatactattttattcaacagatatttattcaatatccactatgagccaggcactggggacacagcagtgagcaaaacaaattccctgaccccatggaattgaccttctagtgggggaaggtattagcaataaatagacaaataagtgtctactacgccagatgggaagaagtggctgtgaagacagagcaaactagagaaacatagagtcaatgtgggatggggtgttcttttaggggggtggtcagggaaagcttatctgagtagttagcttttaagcagagaccccaatgaagaggagggagatatgcgatgcatttagttaggggaagaacattccatgaaaataggatagcaagtgcaaaggccctgagacagcagcatgctttgtgtgttgagggaacagtaaggagaccagtgtggttggtgtgaatggagtgagaaggagcagcaggggttgagggcagaatggtagtgaggagcaggcccttataaaagatgggaagccactggagatctttcaacaaaggggaaaagtatgtttctgttcttgcaatacaatagaaaagcaaaaaatctaggggagttgctaattagccagttttacttatatgccaggtgaaaatatgtggctaggtgcagtggctcatacctgtaattgcagcagtttgggagaccgaagtgggcagatcatctgaggtcaggattcaagaccagcctggccaacatggtgaaaccctgtctctactaaaaattaaaaaattagccaggcgtggtggtgggcacctgtaatcccagctacttgggaggctgaggcaggagaattgcttaaacccgggaggcagaggttgcagtgggccgagactctgtctaaaaaaaaaagaaaatacacattcaggccaggcacagtggctcacgcctgtaatcccagcactttgggaggctgaggcaggtagatcacctgaggtcaggagttcgagaccagcctgaccaacatgggaaaaccctgtctctgccagaaatacaaaaattagccaggcgtggtggtgtgtgcctgtagtcccagctactcgggaggctgaagtaggggaatggcttgaccccaggaggtggaggttatagtgagccaaggttgcaccagcctaggtgacagagtgagactgtctcaaaaaaaaaaaaaagaaagaaaatatacattccatccagaacttgttattctacaagcaaacatcttttattggttagacacccatatatgtgtccctaagcaggaggtggatgccaaataagagacaaatggcgtaagacactatgagttgtgtggtgacattgggcatgtcacttcactccctctgagccttggttagcttctctgtaaaatgaaaggattatggtaactaagctggcttccttccagctttaacaaactgtatggaggtacattttggagttacttgggtaatttttgagtgtgagattggctagaattgctttaatataccaatgtctggccttagcttttggcagagtctgtgtgaagaagcagaggcggagtagagttaattccgtaagttaacgttcagttcgtggcagctggcaatccaaccctgggaaaggctgccggatttagcaaaaatgcaaggtgtctgtttttaaattcgcaatgaattgggtatcctgcattttatttggcaaccctgtcctgggactcacactattcactgttatcactggtatattcgaagtggtgctgacttgccctctgtcttgcaaagtacccgggggtcttttcttatgcttcactggagtcaaaaaagagaatagaggaaaagacaatcatattgttcctttaagagttaagaccaacaagctttcttctttacatgttgtttttgacatgagcaaactggtgattaaaaacaacttgggtggctcatacttgtaatcccagcactttggaaagctgaggtgggagaatagcttgaggccaggagttcaagccagggcaatcctatagtgagaccccatctctacaaaagatacaaaaattagccaggtgtggtggtacacctgtagtcccagctgctccggaggctgagatgggaggatcagttgagcttgggaggcagaagttgcagtgagctgagatcgtgccactgcactccagcctggacaacagagcaagaccctgtctcaaaaaaggaaacaaaacaacttggacaatggaagggggagaaagttcctcaagaagccaaaattgcaccaaatggactcccagaagccaagcatttaacttgttaattgagccctctgtgggcctgtctatacttatttaaggaacaatcctatcaagcatagttattgggtttctcagcccaggtagattagaaatagcagattagaggtgggctaggtttctagaggtaaagtacaccagcagaagttagaagtgaaagcaaagagcctaacagaggaagagaaattcttttttttttttttagacggagttttgctcttgttgcccaggctggagtgcaatggcgctatctcggctcaacgcaacctccgcctcctgggttcaagtgattctcctgcctcagcctcctgagtagctgggattacaggcatgcaccaccacgcccggctaattttgtatttttagtagagacagggtttctccatgttggtcatgctggtctcgaactcctgacctcaggtgatccgcccaccttggcctcccaaagtgctgggattacaggcataagccactgtgcccggccaacaaattcttaaaactggacacaagaacacaaaacgcttgggctgctgagagattagaccaacaaccctccacggctacaaaccttttccacgttatatggcacgttataagtgggtgttcctagtgatggttctgattttttttttaaaaagtctaaatatgtttaatgttgtctcagaagacaaaatatattttagacagatattcctcagtgatgagtaagcctcagctatctggaaaattcatgcaggcgccagagatcattactgagtaattcaagctaataactgcgtcatgctggttgtaccctgcatgccaatatctgctaaaagcagcaccacgaaagggaaatacgaatctcactaagcactcacccattcttgttaacgacactggaactgatcatccttaataatacacagataaatctatcaggagcatttccttgcttcctgtgaaaggaagtactcattccatgtgtcctgtgaaattcagccagcttcgggaagctggaggaatacatatggccaagctacctgggcagagagtagacagggaatggaggttgggcacagtggctcacacctgtaattgcagccctttagaaggcaaaggcgggcagatcacttgagctcaggtgttcaagaccagcctgggcaacatggctaaaccccgtctctgcaaaaaatacaaaaaaatgagctgggtatggtagcacacacttgtggtcccagctacttgggaggctgaggtgggggggttgcttgtgcctgggagtttgaggctgcaatgagctgtgattgtgccactgcactccagcctggataacagaatgagaccctgttccaaaaataaaaaataaaatcaaagacacttaaaaagatggggaaaaggaaggacaggcacttaagcaagttataagctactttcctaactacacaagtggaatcttaagctgaggttcccaggagttgactggagccagagaagacagacctataggagcacccaactggagtcgccctccatagtagcccatatgtcttacatggatcagctttcgtggggcccttctactccgtctggggaagggcgtcagatctgtggctctcatgtactgctcagtacactgccattcccagttctttttttcaaaaaaaaaaaaattgtttacagaatcggccgggtgtggtggcttatgcctataatactagcaatttggaaggctgaggtgggtggatcacctgaggtcaggagttcgagaccagcctggccaacatggtgaaaccccatcctactaaaaaaaaaaaaaaaaaaaaattagctggatgtggtggcaggcgcctataatcttagctacttgggaggctgaggcaggagaatcgcttgaacctgggaggcagaggctgcagtgagccgagatcatgccacggtactccagcctgggtgatagagtgagactctgtctcaaaataaataaaataaaataaaataaaataaaataaaataaaatagtctacagaattaagctggtccaggaatgacagggcgtccatttatttgtctttcaattgtgggagaaaaaggatttctgttgagacactgtcgttttgacacacacaatattttgattaatcttgagattaaaaatcctgtgctccaaatcttttaacattaaattatgcatttaaacaggtttgctcctaaatctcaaaatatggaaagcacctcatgtggctaaatattttgatgaccaagttttctggaaggtaagatttttcacctattaacgtgatagattttgagtgcatgaacttaaaaacatacctgggtatatatgttgacttgctgtttatgagtaaaacaaaaacaaaaatggagtaaggagcattgcaggaggaactagaggagaaacaaatccatgatatgcatgtgtgtgggggagggtggcggggaggtggtaaaggtcaccatttccctgatacctcaaattcattcagagtcagggatgagacagctttcactggccacacttcccctcccgctatctgcagtcctcagcgtagccaaatagtttgacatgcgggtgacagaaccccgcaatgcaaaagctggaagaaacctcaagccttggagtccaaccccttttttgacagatgctaagagtggagacatgacttatcaagatcttacaactggctgggcacggtggctgacgcctgtaatcccagcactttgggaggctgaggtggggcgatcacctgaggccaggagttcgagaccagcctggccaacgtgtcaaaaccccatctctactaaaaatacaaaagttagctgggcgtggtagcacatgcctgtaatcccagttactcaggaggctgaggcaagagaatcgcttgaaatcaggaggcagaggttgcagtgagctgagattgcgccactgcactccagcctgggtgacaagagctgacactctgtctcaaaaaaaaaaaaaaaaaaaaaaaattcttacagtgtgtgagtatccaggctgagtcctgaacacagctcttgataaatgataacaagcaggcacaaaaaaattgtagtacaggagtctgaggtcacttagcaaagggacataaagttcaaacagctcagcagctgctgagggtcccgtgttacattgtagcatttgttgttgtgactgggctagaaagaaggtgaagaaggttggagctcactccctgcctcccctcccactctcctccctttgacctacactcatagttcacgcagcactctgatgtgtccccttaggccatcctctagtcaatgctgtgggtaggctggaccagcagggaccagtattgtcacagcaagtccaggccaacagtggtcaggctgctgcccggtgttgtgcctttgtgagtggcagatccaagaccggaacccaggccttctgagtcccaggccaatgcttgccccacccagcatccaagatgttgctcactaaagagacagagaagcagccttattatgggcctggacacctgtgcatgaggggtcaagcagagaggacctggggagagaccctgccccttcttttccttctccttcctctcctttctcttcttcttcctcttcaaatagctttttgaggtgtaactggcatacaatcaattgtacatatttaggctgggtatggtggctcacgcctgtaatcccagcactttgggaggccaaggcgggtggatcacttgaggtcaggagtttgagaccagcctgggcaacccggtgaaaccccgtctccactaaaactacaaaaattagccaggcgtggtggcagctgcctgtaatcccggctactcgggaggctgaggcaggagaatcacttgaacctgggaagcgaagtttgcagtgatctgagatcatgccactgcactccagcctgggtgacagagcgagactttgcctcaaaaacaaaacaaaacaattgtacatatttaaagtgttgtaaccaagtgagttacagagaaacaccacactttgagcctaattcaggagtcctttattagccggcgacctagagacgactagtgctcaaaattctctcggccccaaagaaggggctagattttcttttataccttggtttagaaaggggagggggaattgagctgaagcaatcttacagaagtaaaacaggcaaaaaagttaaaaagacaaatggttacaggaaaacaaacagttccaggtgcaggagctttaaagccatcacaaggtgacaggtgcgggggctctgggtgctatctgccggacacaaacgcaggggcactagagtactatcacccgggcaaattcctgggaactgcggacacagcttgccacagtaccttatcagctaattgcactctttgatgtgctgggagtcagcttgcacaagttaagtccttgaggaagggggtgggtaaggagcccttaacgtcttgcaaatgaaggagccgaatggaatccctccggctttcttagctaagagagagtcaatcaagttaatacaagttagggtatcacaaaagtatataatttgatacattttaacgtatttatacactgaagagaccatcaccaccatcaagacaaggagcacacccatcacttccacacacttcctcctgctcctttgaaattcctcccttcctacccacctggtcccacccaaaggcaaccactgaactactttctgtcactaaggtttgcgttttctgtaatttttttgtttgagacagggtctcactccgccacccacaccgtaatgcagtggcaccatcatgactcactgtagcctcaacctccccaggctcaggagatcctcccccctcagcctcctgagtagctaggaccacaggtgtaggccaccatggcaggctaatttttgtatttttttgtagagatggggtttcaccgtattacctaggctggtctcgaactcatgggttcaagcaatcctcctgccttggcctctcaaagtgctgggattataggcatgagccactgtgcccagccctctgtaatgttacacaaagggaatcatgcagcacgtactgcccttggtctggcctcttttgctcagcatgattattctgagaatcatccgtgttgttgcgtgtaactgacttcatcagcttctctctgcagctgtcagctcttggcttctcccaacagccaatctctctttatcccctgcaagtgttcttgcctatttagcagaatcaaggtactctatcgaaaagactcggaaaattggtttaatctattcattcattcctcaggtatttatcgaataactattctataccaagtactatgctaatcaaccaaggacagcacaaacaggagaaatctccagctcagtcacttgagttgcaataaatatttgctggataggtcaggtgcagtggctcacacttgtaatcccagcactttggggattactgagacgggaggatctcttgagcccaggaggccaaggctgcagagaaccatgatcatgccactgcactccagcctgggtgacagagtgagatcctgtctctgaaaaaaaatatttgctggataaattaaggaaatctgacgaaccccatcagtagccattgcagcaacaggtaaactagaacgagtgtgaatttggaatgaggaaacccgatgttggccatcattctgtaatgtcatgtattatgtaatgtattatatattaatgtatgtattatgtaggcaagttccttgacctctctcactggtaacataagagtagtaatctttgtgctacttcactgggttatttcaaagatcaagtgaggtaataatgtctgtaacaacattctgtaaaatgcaaaccgccacatgaatgtgaaagtttattactagggatttagccaaccacaagggaatgtgtgagcataagagctatcatattgcaagcctacagtttctgattttgtgctaggtgcttttccacattacctgattttatcctcacaacagtcctgcataaaagtaagtatgtcgcccaggtgcggtggctcatgcctataatcccagcactttgggagcccgaggtgggcaaatcacttgagatcaggagtttgaaaccagcctggtcaacgtggtgcaaccctgtctctactaaaaatacaaaaaaaaattagacaggcgtggtggtggatgcctgtaatcccagctacttgggaagctgaggcaggagaatggcttgagcccgggagatggagattgcagtgagatgagattgcgccactgcactccagcctgggtgacagagcaaggctatgtctcaaaagagaaaaaaaaagtaagtatctcagtcttgaagatgatgaaatggaggcctagagagattaagtaacttgcccaaaatgacagaactaatgcatagaaaagaagaaatgtgatgtcttttggctccaaagacaccccacatatgcgttggttacagttactagagaaaagttattccacccccaccccacccccagaaatcttctgacttgttttctcgcagttgagtaggaccatttattcggcagtgtaccattctcagcttgcagttgaaagccaaatatccattaaagaggcaaggatgcaaacttgctaagctgataaatccaggggtgattttttttttttttgcaaaccatccaacaagacattttaaatactcattgaatttcatagaactgactgccaggattggaaagacattaaagccagctcagccactgcctcgctggttggccagaccacgcctggcacttctgggagggagcactcaccaccccccaagggcacccatctcatcctccgaaggtttatgaaaatgcactcatcatttgctaattcattccactacgtgtattacctaatttgtgacacgatgtgaagtaccagagagataattctaaataaaatatagttatgggtctcaaggagccagatatgctaatctcctatcctcctgcagtttacagtggtcctcaccagatacttatttacaaaaattcagtttattatttatttttttgagacagagtcttgctctatagctcaggctagagtgtaatggtgtgatctcggctcacttcaacctctgcctcccaggttcaagtgattctcctgcctcaacctcccaagtagctgggactacaggcacctgccaccacggctaatttttggagttttagtagagacagggtttcaccacgttggccaggctggcctcgaactcctgacctcaggtgatctgcccacatcagcctcccaaaatgttgggattacaggcgtgagccaccatgcccggccaaaacttcagtttataacacaatctttcacgtgtcttctgctttcattaaaagaatagacagttcccttctttatttcagtttaataaaccatggattttatttcatgctttgcaaaacacaagggctcactgacatgcacttcttaaactaattctggctggtcgcctgtaattccagcactttgggaggctgaggccgacagatcacttcaagtcaggagttcaagaccagcctggccaatatggtgaaaccacgtctctaccaaaaatataaaaaattagccaggtgtggtggtgcgtgactataatcccagctactcaggggcctgaggcagaaaaatcacttgaacccgggaggcggaggttacagtgagctgagatcgcgccactgcactccagcctgggcgacagagtgagactctgtctcaaaaaataaataaatacaaataatgtaaaatacgaaacaagcaatcctggcagtagctgctggaatgagaggagggagaggtcatagggaggtcggggacaatggagcatggagttgtgttggatttggctaagcagcaggaagtgcaaggcattccaagcaagaggaggggggcaggtggggagcatctgcaagaacagaagcagcatgagcaacctggctcggcagtgtgtgaaaaggctgaaaggtggctagagccacttcaatttcatccttcaggcaaatgggaaattcccaaaggtttgagtggggaagcaatgcctacaatgaaagtttgagagtgaagcagagtgatcgaattaagcatgtaggccgagttctgaaataactgcaatgtgctgaagatcatccattggcttctgaatgagtatttgcagtttattttttaaaatgattttattgccaagaaagataaacactactgttttggtacaaaaacataacaaaatgtgttgagtccctcttgctgttttacgcgaagttttaaaaatctactcttgtcacagtggtatcacccctacttctgatttcaaataaatgttctagagacacagtaagggcccaacaaacgcttgttcaacaacacaaggagagccagcttttaaagtaggaaaacaggccgggcgccgtggctcacacctgtaatcccaacactttgggaggctgaggtgggcagatcacttgaggtcaggagttcaagaacagcttggccaacatggtgaaaccctgtctctactaaaaacacaaacattagccaggcgtggtggtgcacaccagtagtcccagctattcaggaggctgaggcaggaaaatggcttgaactggggaggcagtggttgcagtgagccgagatcgtgccactgcactccagcctgggggacagagggagactccatctcaaaataaaacaaaacaaaaccaaatcatacaaaaacattagctgggtgtggtggtgcatacctgtaatcccagctacttgggaagctgaggcagaattacttgaacccctggggggaggttgcagtgagctgagatcttgccactacactccagcctgggcaacagagtgaggagactctgtctcaaaaaatatatatattaaaaaaaagaaaaaaaaaagtaaactaggaaaacacatcagcagcctgccaacagactcccctagcctcggtgagggccagtgttctgggaggcagatctgaattctagtcctagttcacccactggcaggctggtgcccttgggcaggtcgcttctctggggctcagtttcttcctctataaaatgagatcaaatcccatgttctaagagtttgtgctctggagtcagacagatctgggttctaccactgccagctctgtgatcttgtagcttcagtctcgtcatctgacatggagataacagtaactgtctcactgtgttgttagggtttaaaggagataatgtatgtgaaatgttagcaaacaagtgttagctaccctgatttccggtttcagagttctgtggtcccagtttatgccacatgcagtgacgttgtatggtaggctgtggtgtggcaccacttcagaactcagcgcatgcacagcttgcagaagagaaggccagaggagacctaagaaggctcttcgaacacttgaaagaccggcatgtaggccgggcgcagtgactcacgcctgtaatcccagcagttttggaggtcgaggcgggtggatcacctgagtttgggagtttgataccagcctgaccaacaaggtgaaaccccgtctctactaaaaaatacaaacattagctgggcatggtggcgggtgcctgtaatcccagctactccggtggttgaggcagaattgcttgaacccgggaggcagaggttgcagtgagctgagattgcatcactgcactccagcctgagacaagagcgaaactccatctcaaacaaaacaaacaaccaaccaaacaaaaccaaaaaaaaaactggcatgtagaagaaaaatactttttctctacacttctccaaagaatttaactaggcccaggggaggtgcagtataaatttctaacaatctcaactgtctgccaaatggaatgagctacttcatatggcagtagtgagtcctctgtctttggaggcattcaaataaaagccagatggccatttatcaacaatccatgtaaaacgttagatgaaataaaacctatatatccaagatctcttccaattcagattttatgaaagaatttctaaggtctttgtaatgagacatttaggctgtttcaagagatcaagccaaaatcagtatgtgggttcatctgcaataaaaatgtttgttttgcttttacagtttcctcatttggctgttggattttaagcaaaagcatccaagaaaaacaaggcctgttcaaaaacaagacaacttcctctcactgttgcctgcatttgtacgtgagaaacgctcatgacagcaaagtctccttatgtataatgaaacaaggtcagagacagatttgatattaaaaaattaaagactaaaaacttagtttaagagtcaatttaataagtttaaaataaatgtttagtttcattaggatgatgctatcaatattttcttggttacagacacattattaaagttttgggttaattttattgacaattcttaagattctttctcatgcttaataaagcatgctactcagttaactcttgtctacatcagcaaagcagataatacaaaacaggaaaattacaaatcactgatacttagtccttgtgggaatcatgcttttctcccagcagttttacaaggtggctggcattccctgagcatattctgaattgcactgtggggaaagaggttgtgctcagttgtagggtggggggatgcactgcctgaggattaaaaaactagttctgtgaccgtgaggaagtcgtttaaatttccatggtctgttccctcctatgtgaaaagagaaggtgggcttcaacctctaagatcttctccagttttcacattttatggacttttgtagaaaaaacatcaggagttcatgtgggatgacagcaagtcatttctttgaggagagtcttgatcaccaggcaatattcacagtgtagagactgtcagatgaccatggctagcatggaaatgagacccacacatttaaatcacccagcaaatattccgaaggctaattgtagcacattttatgaaagacatttcaaactgtggtcctgaagagtgtatcccatcttgcagaggtggggagcctggggggacaagagttctgaagaggaagagacaacaagagttcccagtagctaatgtttgtcattctagttgaccgtgctggtctattaggctagtggttcagtacacagatgaaatgcaacatggaacccagtttattatcagaacaactacaaagaaattgtcccctgtctaagactggagtgtcaagtctctgcccttttttcctttccttcaatggtggatgtggagtgactgtgcatcccaccagaaccacgtgtcatggctgagtcacatcttcctgcccttggaatgagaggcacagcggaagaccttcccatggaagggacacagggagcctggtggctggaccatggtgcttctctcttccaacacgtccactcaccccttgggagaccctcaaaagccagttacattacatgttcacagaatttttggtaaaagtaaataccaattatagtgaggaagaattttgaccacggaatattttaaaaactaaaaaatgtttatatttcatttaacatttgacacagaagagaccacatttgaataaacacattaaatcttcagagcactttcattgtggttttggacctcagatatgacaaatacttacattgacaaatccataatttcttttgtaatttctttttatttttacaaattataccatgataaaatttgacaaaaattattcatgtgaaagtttcctctaacattttataagttaatcaagtgcataccacaatagatttttggttgttgtttaggtgttctcgtgattttagtattacacaactttaagctgagactacactcagaaataagtttagaaaatggcattacaaaaggttgggagtgagcagtaaaaaaacaaacaaacccatgcagggctgttgtgctgtgggaaatcagatgtgttcactgccataagtcttcagtgcggccaaacttaaaaaccagccctctgtgaataaaacaagaaatatcacatgactccctgaatttgagaaaagagtatgtgagatttcgagaatggtgtgaaacaaacaacgaagaataattgatgagttgtagaagaaattttggtacgaaatgtatcaaaacagaaactgatcattctaaggtagtgaattcttccattatgttcaactgtgctattaaccaccatattcccaacaaccttaactttcaagtactgaatacacatgtgacttttaaaaagttaccagtgtttactatgtaaccattatatgtctgatttttttttttttttttgagacagagtcttgctctgtcgcccaggctggagtgcagtggcgtgatctcggctcactgcaagctctgcctcccgggttcatgccattctcctgcctct

(19) United States
(12) Patent Application Publication
(10) Pub. No.: US 2012/0172239 A1
(43) Pub. Date: Jul. 5, 2012

(54) METHOD FOR THE IDENTIFICATION BY MOLECULAR TECHNIQUES OF GENETIC VARIANTS THAT ENCODE NO D ANTIGEN (D-) AND ALTERED C ANTIGEN (C+W)

(75) Inventors: Jorge Ochoa, Derio (ES); Monica Lopez, Derio (ES); Diego Tejedor, Derio (ES); Antonio Martinez, Derio (ES); Laureano Simon, Derio (ES)

(73) Assignee: Progenika Biopharma S.A.

(21) Appl. No.: 13/339,058

(22) Filed: Dec. 28, 2011

(30) Foreign Application Priority Data
Dec. 31, 2010 (EP) 10197481.4

(51) Int. Cl. C40B 20/00; C40B 20/08; C07H 21/04; C40B 40/06; C12Q 1/68; G01N 21/64

(52) U.S. Cl. 506/6; 435/6.11; 536/24.33; 506/16; 506/2

(57) ABSTRACT

The invention relates to genotyping and blood cell antigen determination. In particular, the invention addresses discriminating the RHD*DIIIa-CE(4-7)-D or RHD*DIIIa-CE(4-7)-D)-like blood type variants, from RHD*DIIIa, RHD*DIVa-2 and other blood type variants. The invention provides methods for genotyping a subject, comprising:
a) determining at least 4 markers in a sample that has been obtained from the subject, wherein the markers comprise:
(i) the presence or absence of an RHCE*C allele;
(ii) the presence or absence of an RHD/RHCE hybrid exon 3 (RHD/CE Hex03) allele;
(iii) the absence of, or a single nucleotide polymorphism (SNP) variant within, any one of position 602 of exon 4, position 667 of exon 5, or position 819 of exon 6 of RHD; and
(iv) the absence of, or SNP variant within, position 1048 of RHD exon 7. The invention also provides probes, primers and kits for use in such methods.

### CROSS REFERENCE TO RELATED APPLICATION

This claims the benefit of European Patent Application No. 10197481.4, filed Dec. 31, 2010, which is incorporated by reference herein in its entirety.

### FIELD OF THE INVENTION

The invention relates to methods for genotyping and blood cell antigen determination, which in particular may discriminate the RHD*DIIIa-CE(4-7)-D or RHD*DIIIa-CE(4-7)-D)-like blood type variants, which express the C^{+W} antigen and lack a D antigen, from RHD*DIIIa, RHD*DIVa-2 and other blood type variants. The invention also relates to products, in particular, probes, primers and kits for use in such methods.

### BACKGROUND TO THE INVENTION

The success of blood transfusion often depends on the degree of compatibility between donor and recipient. The degree of compatibility, in turn, is a function of the similarity in Red Blood Cell (RBC) antigen content between donor and recipient. Most RBC antigens in an individual can be predicted in a simple manner from the analysis of their genomic DNA. Therefore, analysis of donor and/or recipient DNA can be used to predict the degree of compatibility and thus enable proper blood transfusion practice.

Hemolytic reactions are more common in multi-transfused than in singly transfused individuals, not only because of the increased probability of such an event as the number of transfused units increases, but also because of the accumulative immunological memory-driven nature of the immune response in the recipient. An example of a condition whose treatment includes repeated blood transfusions is Sickle Cell Disease (SCD). It therefore follows that a high degree of compatibility with donor blood is often critical for the success of transfusion in SCD patients.

While SCD is more prevalent among individuals of African ancestry, the blood donor population in the USA and other Western countries is largely Caucasian. As a consequence of this disparity, differences in RBC antigens between both racial groups often become responsible for blood transfusion failures in SCD patients.

The genetic variant RHD*DIIIa-CE(4-7)-D (also known as RHD-CE-D^{S}, RHD-CE(4-7)-D, (C)ce^{S}, or r′^{S}) can be found in approximately 5% of the African-American population, but has not been reported in Caucasians. This variant poses a special challenge to blood transfusion because it encodes a rather complex antigen profile, which includes absence of D antigen, altered forms of C (C^{+W}) and e antigens, expression of low-frequency VS antigen, no expression of V antigen, and absence of the high-frequency hr^{B} antigen. The D and C antigenic profiles are the clinically most relevant for determining compatibility between blood donors and transfusion patients.

The antigenic complexity of RHD*DIIIa-CE(4-7)-D correlates with its genetic complexity, which includes changes to the RHD gene, with a substitution of part of RHD exon 3, RHD exons 4-7, and the intervening introns by their RHCE counterparts, a G>T substitution at position 186 (exon 2), a C>T substitution at position 410 (hybrid exon 3), a C>G substitution at position 733 (exon 5), and a G>T substitution at position 1006 (exon 7). In addition to the changes in the RHD gene, RHD*DIIIa-CE(4-7)-D occurs in cis with an RHCE gene that encodes substitutions C>G at position 733 (exon 5) and G>T at position 1006 (exon 7). The C^{+W} phenotype is characterized by weak expression the Rh C antigen.

To add to the antigenic and genetic complexity, our knowledge about the molecular basis of RHD*DIIIa-CE(4-7)-D is incomplete. For instance, the precise points of RHCE/RHD recombination in intron 3 or intron 7 have not been reported to date. Furthermore, two types of RHD*DIIIa-CE(4-7)-D variants have been described (Type 1 and Type 2), which differ in both their genetic composition and antigen profiles, although the clinically relevant D and C antigenic profiles are the same. There may be other RHD*DIIIa-CE(4-7)-D—like phenotypes, which also have a D− and C^{+W} antigenic profile. Clinically, it is therefore most important to distinguish RHD*DIIIa-CE(4-7)-D and other RHD*DIIIa-CE(4-7)-D—like phenotypes from phenotypes with different C and D antigenic profiles.

Several publications (Refs. 1-3) have examined the genetic similarity between RHD*DIIIa-CE(4-7)-D and other RHD variants, in particular RHD*DIIIa and RHD*DIVa-IIRHD*DIVa-2 (RHD*DIVa-2 henceforth). A number of molecular methods for the specific detection of RHD*DIIIa-CE(4-7)-D relied on the detection of single nucleotide polymorphisms (SNPs) located in hybrid exon 3. These SNPs are now known to be shared with variants RHD*DIIIa and RHD*DIVa-2. Consequently, identification of RHD*DIIIa-CE(4-7)-D in a sample by DNA analysis requires detection of hybrid exon 3 SNPs and discrimination from RHD*DIIIa and RHD*DIVa-2, which is difficult with current methods of genotyping. This discrimination is clinically relevant since RHD*DIIIa and RHD*DIVa-2 encode a different antigen profile, which includes expression of partial D and absence of C^{+W} (i.e. partial D, C−). It is also important to distinguish between other genetic variants that may also share these hybrid exon 3 SNPs but encode different combinations of D and C antigens, which may also be clinically relevant.

Some of these other RHD variants have been identified, for example RHD*DIVb-4, RHD*weakDtype4.0, RHD*weakDtype4.1, RHD*weakDtype14, RHD*weakDtype51, RHD*DAR, RHD*DAR-E, RHD*ex04-ex07del, RHD*ex03del and RHD*ex03-ex04del, which have varied expression of D antigen.

Antibody reagents commonly used to detect C antigen do not discriminate between C^{+W} and C. Therefore, the phenotype is often reported as C. In cases where the antibody reagent does discriminate between C^{+W} and C⁺ but the sample contains a normal RHCE*C allele in trans to a RHD*DIIIa-CE(4-7)-D allele, C^{+W} is obscured by C⁺, resulting in a C⁺ phenotype for the sample. This makes it difficult to determine the correct phenotype for C^{+W}/C⁺, C^{+W}/C^{+W}, C^{+W}/C⁺ and C⁺/C⁺ antigenic profiles using serology analysis alone. Therefore, RHCE*C needs to be tested for and shown absent prior to assignment of a C^{+W} phenotype to a sample, and so current methods of diagnosing a RHD*DIIIa-CE(4-7)-D antigenic profile are difficult even when both serology analysis and genetic analysis are performed.

### SUMMARY OF THE INVENTION

The inventors have found a method of discriminating the RHD*DIIIa-CE(4-7)-D or RHD*DIIIa-CE(4-7)-D)-like blood type variants, which express the C^{+W} antigen and lack a D antigen, from RHD*DIIIa, RHD*DIVa-2 and other blood type variants, by determining at least four genetic markers. The method does not require the use of antibodies, and enables prediction of the D and C antigen phenotypes of a large majority of samples containing RHD/RHCE hybrid exon 3. At its most general, the method comprises: (1) Determining the genotype of a sample, (2) Deducing from the genotype whether or not the sample contains a RHD*DIIIa-CE(4-7)-D haplotype and whether or not it contains other haplotypes than may affect in trans the phenotype encoded by RHD*DIIIa-CE(4-7)-D, and (3) Predicting from the haplotypes whether or not the phenotype of the sample is D⁻, C^{+W}. The genotype determinations may be made using the following markers:
i) presence/absence of at least one RHCE*C allele,
ii) presence/absence of at least one RHD/RHCE hybrid exon 3 (RHD/CE Hex03) allele,
iii) presence/absence of, and SNP variant within any one of the following exons: RHD exon 4, RHD exon 5, and RHD exon 6, and
iv) the presence/absence of, and SNP variant within, RHD exon 7.

Preferably, the SNP variant within RHD exon 4 is at position 602 of the RHD coding sequence (rs1053355), the SNP variant within RHD exon 5 is at position 667 of the RHD coding sequence (rs1053356), the SNP variant within RHD exon 6 is at position 819 of the RHD coding sequence (rs number not available), and/or the SNP variant within RHD exon 7 is at position 1048 of the RHD coding sequence (rs41307826). In some cases, the SNP variant within RHD exon 7 is at position 1006 (rs number not available).

The method of discriminating the blood type variants may be further improved by determining at least five genetic markers, wherein the fifth marker is:
v) presence/absence of at least one RHD exon 3 allele.

Different C and D antigen phenotypes can then be discriminated because combinations of the four or five markers above are unique to either one of the RHD*DIIIa, RHD*DIVa-2, and RHD*DIIIa-CE(4-7)-D or RHD*DIIIa-CE(4-7)-D)-like blood type variants, as shown in Table 1. A method of the invention may therefore further comprise a step of using the marker determinations to assign a C and D antigen phenotype, for example based on the correlations shown in Table 1. The methods of the invention therefore provide considerable efficiency savings in comparison with, for example, full sequencing or genotyping of a large number of polymorphisms, or genotyping samples in combination with serology analysis.

The invention therefore provides a method for determining the presence or absence of RHD*DIIIa-CE(4-7)-D or RHD*DIIIa-CE(4-7)-D)-like blood type variants, wherein the method comprises determining at least four markers, wherein the markers comprise:

(i) the presence or absence of an RHCE*C allele;
(ii) the presence or absence of an RHD/RHCE hybrid exon 3 (RHD/CE Hex03) allele;
(iii) the absence of, or single nucleotide polymorphism (SNP) variant within, any one of RHD exon 4, RHD exon 5, or RHD exon 6; and
(iv) the absence of, or SNP variant within, RHD exon 7.

Preferably, the SNP variant within RHD exon 4 is at position 602 of the RHD coding sequence (rs1053355), the SNP variant within RHD exon 5 is at position 667 of the RHD coding sequence (rs1053356), the SNP variant within RHD exon 6 is at position 819 of the RHD coding sequence (rs number not available), and/or the SNP variant within RHD exon 7 is at position 1048 of the RHD coding sequence (rs41307826).

In a first aspect, the present invention provides a method of genotyping a subject, the method comprising:

a) determining at least 4 markers in a sample that has been obtained from the subject, wherein the markers comprise:
(i) the presence or absence of an RHCE*C allele;
(ii) the presence or absence of an RHD/RHCE hybrid exon 3 (RHD/CE Hex03) allele;
(iii) the absence or single nucleotide polymorphism (SNP) variant of any one of the SNPs: within RHD exon 4 at position 602 of the RHD coding sequence (rs1053355); within RHD exon 5 at position 667 of the RHD coding sequence (rs1053356); or within RHD exon 6 at position 819 of the RHD coding sequence (rs number not available); and
(iv) the absence or SNP variant of the SNP within RHD exon 7 at position 1048 of the RHD coding sequence (rs41307826).

In some embodiments, the method further comprises determining the marker:

(v) the presence or absence of an RHD exon 3 allele.

Preferably, the method further comprises determining the RHD and RHC antigen phenotypes of the subject.

In some embodiments, the method comprises detecting the presence or absence of a blood type variant selected from: RHD*DIIIa; RHD*DIVa-2; or RHD*DIIIa-CE(4-7)-D or RHD*DIIIa-CE(4-7)-D)-like blood type variants. Preferably, the method comprises detecting the presence or absence of RHD*DIIIa-CE(4-7)-D or RHD*DIIIa-CE(4-7)-D)-like blood type variants.

Preferably, marker (iii) is the SNP within RHD exon 4 at position 602 of the RHD coding sequence (rs1053355).

Preferably, RHCE*C allele is determined by determining the presence or absence of RHCE*C intron 2. In some embodiments, the RHCE*C allele is determined by determining the presence or absence of any one of the following positions in the RHCE coding sequence: position 307 (exon 2), position 48 (exon 1), position 150 (exon 2), position 178 (exon 2), position 201 (exon 2) and/or position 203 (exon 2).

In some embodiments of the method described herein, the sample comprises nucleic acid and the method comprises amplifying the nucleic acid or a portion thereof by PCR using primers. Preferably, different portions of nucleic acid are amplified simultaneously using multiplex PCR.

Preferably, the PCR primers for determining the RHCE*C allele are a forward PCR primer specific for RHCE*C, and a non-specific reverse PCR primer. The non-specific reverse PCR primer may be shared with RHD, RHC*C and/or RHC*c.

Preferably, the PCR primers for determining the RHD/CE Hex03 allele are forward and reverse PCR primers targeting sequences located in introns 2 and 3, or introns 3 and 2, respectively.

Preferably, the PCR primers for determining the SNP at position 602 of the RHD coding sequence, within exon 4, are forward and reverse primers targeting sequences located in introns 3 and 4, or introns 4 and 3, respectively.

Preferably, the PCR primers for determining the SNP at position 667 of the RHD coding sequence, within exon 5, are forward and reverse primers targeting sequences located in introns 4 and 5, or introns 5 and 4, respectively.

Preferably, the PCR primers for determining the SNP at position 819 of RHD coding sequence, within exon 6, are forward and reverse primers targeting sequences located in introns 5 and 6, or introns 6 and 5, respectively.

Preferably, the PCR primers for determining the SNP at position 1048 of RHD coding sequence, within exon 7, are forward and reverse primers targeting sequences located in introns 6 and 7, or introns 7 and 6, respectively.

Preferably, the PCR primers for determining the RHD exon 3 allele are forward and reverse primers targeting sequences located in introns 2 and 3, or introns 3 and 2, respectively.

In some embodiments, the nucleic acid containing the amplified marker (amplicon) comprises a label. Preferably, the label comprises a biotinylated nucleotide. Preferably, the label comprises a fluorescent moiety.

In some embodiments, the sample comprises nucleic acid, and the method comprises amplifying the nucleic acid or a portion thereof by PCR using primers, fragmenting the amplified nucleic acid, and labelling the fragmented nucleic acid with biotinylated ddNTPS using a terminal deoxynucleotidyl transferase (TdT) enzyme.

In a further embodiment, determining the presence, absence or SNP variant of a marker described herein comprises contacting nucleic acid containing each marker with one or more probes. Preferably, nucleic acid containing each marker is amplified by PCR as described herein before contact with the one or more probes.

Preferably, the probes for determining the presence or absence of RHD/CE Hex03 or RHD exon 3 contact an SNP located in both RHD/CE Hex03 and RHD exon 3, wherein one SNP variant is specific for RHD/CE Hex03, and another SNP variant is specific for RHD exon 3. Preferably, the SNP is at position 410 of the coding sequence, located within both RHD/CE Hex03 and RHD exon 3 (rs number not available).

Preferably, one or more of the probes comprise a label. Preferably, the label is a fluorescent moiety. Preferably, one or more of the probes are immobilised on a solid support or conjugated to one or more particles.

In a second aspect, the present invention provides a set of primers for amplifying nucleic acid comprising at least four (e.g. five) of the markers described herein. Preferably, the set of PCR primers comprise forward and reverse PCR primers targeting sequences in the locations described herein.

In some embodiments, the set of primers comprises at least three primer pairs selected from the primers described herein for the markers described herein. Preferably, the set of primers comprises four or five of the primer pairs described herein for the markers described herein.

In some embodiments, at least 50%, e.g. 60%, 70%, 80% or 90%, of primer pairs in the set are primer pairs described herein.

In a third aspect, the present invention provides a set of probes for determining the presence, absence or SNP variant of at least three, four or five of the markers described herein. Preferably, the probes target the locations described herein.

In some embodiments, the set of probes comprises at least 60%, 70%, 80%, 90% or 100% of the probes described herein for each marker.

Preferably, the probes comprise a label. Preferably, the label is a fluorescent moiety.

Preferably, the probes are immobilised on a solid support or conjugated to one or more particles.

In a fourth aspect, the present invention provides a kit for genotyping a subject, the kit comprising a set of PCR primers described herein and a set of probes described herein.

The invention will now be described in more detail, by way of example and not limitation, by reference to the accompanying drawings. Many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

### DESCRIPTION OF THE FIGURES

FIG. 1: Coding sequence of RHD (SEQ ID NO: 1), showing the positions of each exon. The nucleotide sequence shown is a consensus sequence.

FIG. 2: Coding sequence of RHCE (SEQ ID NO: 2), showing the positions of each exon. The nucleotide sequence shown is a consensus sequence.

### DETAILED DESCRIPTION OF THE INVENTION

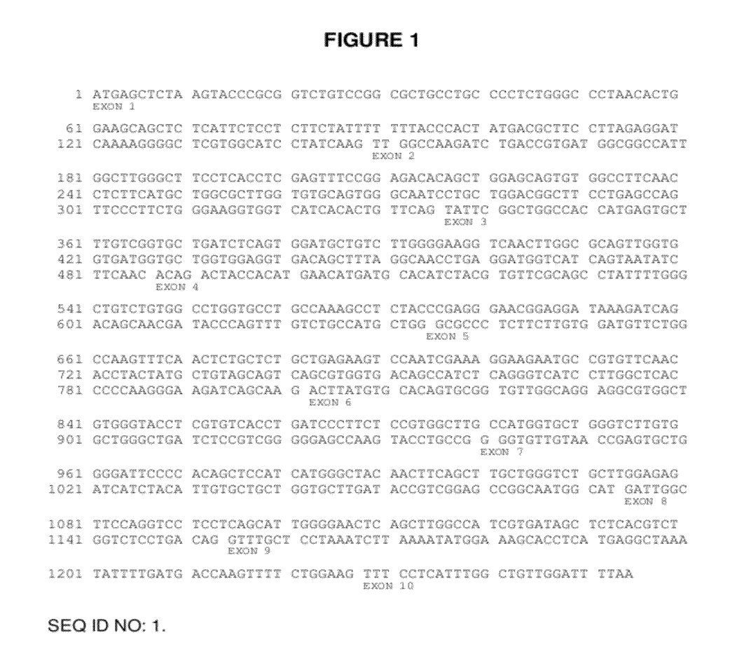

The Rh blood group D antigen is encoded by the RHD gene, which comprises 10 exons. The complete RHD gene sequence is available at NCBI Reference Sequence: NG_{—}007494.1 No. NG_{—}007494.1, GI:171184448, (SEQ ID NO: 41), the entire contents of which is incorporated herein by reference. The coding sequence, annotated to show the starting position of each exon, is shown in FIG. 1 (SEQ ID NO: 1).

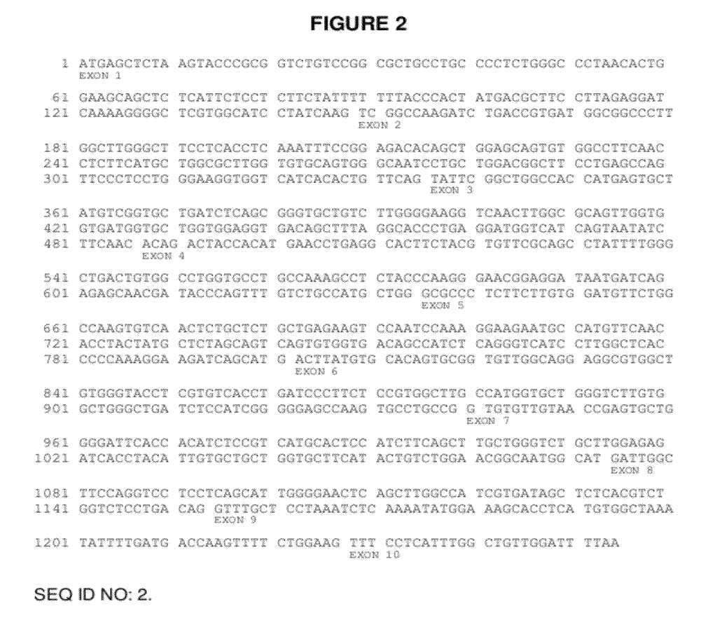

The Rh blood group C antigen is encoded by the RHCE gene, which comprises 10 exons. The complete RHCE gene sequence is available at NCBI Reference Sequence: NG_{—}009208.2, GI:301336136, (SEQ ID NO: 42), the entire contents of which is incorporated herein by reference. The coding sequence, annotated to show the starting position of each exon, is shown in FIG. 2 (SEQ ID NO: 2).

The present invention enables determination of the clinically relevant RHD and RHC antigen phenotypes of a blood sample, based on at least the following four markers: at least one RHCE*C allele; at least one RHD/RHCE hybrid exon 3 (RHD/CE Hex03) allele; the SNP at position 602 of the RHD coding sequence, within exon 4, or the SNP at position 667 of the RHD coding sequence, within exon 5, or the SNP at position 819 of the RHD coding sequence, within exon 6; and the SNP at position 1048 of the RHD coding sequence, within exon 7.

Determination of the clinically relevant RHD and RHC antigen phenotypes of a blood sample can be further based on determining a fifth marker: namely, at least one RHD exon 3 allele.

Table 1 demonstrates how the combination of these five markers enables the prediction of the vast majority of RHD and RHC antigen phenotypes. The first three columns show whether at least one allele of RHCE*C, RHD/RHCE hybrid exon 3 (RHD/CE Hex03) allele, and RHD exon 3 are present. The columns entitled RHD 602 and RHD 1048 show the SNP variants at these positions, i.e whether they are absent, heterozygous, or homozygous/one SNP is absent. The presence of at least one allele of RHCE*C determines whether a C⁺ antigen is present, and the next four columns provide information on the D antigen phenotype, and C antigen phenotype in the absence of RHCE*C. Together, these data enable the RHD genetic haplotypes and D and C antigenic phenotypes to be predicted, and different blood types can therefore be distinguished on this basis.

Although the combination of the 4 or 5 markers described herein are able to distinguish RHD*DIIIa-CE(4-7)-D or RHD*DIIIa-CE(4-7)-D)-like blood type variants from RHD*DIIIa, RHD*DIVa-2 and other blood type variants, they are not intended to unambiguously predict all possible C and D antigenic combinations. Therefore, “Possibly RHD” in the haplotype column is a generic term meant to include RHD as well as RHD variants other than the ones interrogated by the present method. Likewise, “D?” in the phenotype column refers to D, Partial D, Weak D, D− or Dₑ₁ phenotypes encoded by RHD variants other than those interrogated by the present method. D+ is the most likely phenotype in this situation, however.

Interpretation of the Predicted Phenotype data in Table 1 is facilitated by Table 2, which provides an exhaustive description of how the haplotype-encoded phenotypes relevant to the present invention (the D antigen phenotype and C antigen phenotype) combine to yield the phenotype of a sample.

In some embodiments, presence/absence of RHD exon 3 can be determined by determining the SNP nucleotide sequence at position 410 of the RHD coding sequence, within exon 3 (rs number not available).

In some embodiments, determination of the nucleotide sequence at position 602 of the RHD coding sequence, within exon 4, could be substituted by the determination of the SNP nucleotide sequence (nucleotide T vs. nucleotide G) at position 667 of the RHD coding sequence, within exon 5, or the SNP nucleotide sequence (nucleotide G vs. nucleotide A) at position 819 of the RHD coding sequence, within exon 6.

Other combinations of markers that include fewer than the at least 4 or 5 markers described herein would result in a decreased power to determine D⁻C^{-W} phenotypes.

For instance, without the determination of the presence/absence of RHCE*C, it would not be possible to predict a C⁻ phenotype for RHD*DIIIa and/or RHD*DIVa-2 samples or a C^{+W} phenotype for RHD*DIIIa-CE(4-7)-D samples.

Without the determination of the presence/absence of RHD/RHCE hybrid exon 3, it would not be possible to deduce a RHD*DIIIa-CE(4-7)-D haplotype for any sample, and therefore it would not be possible to predict a C^{+W} phenotype for any sample.

Without the determination of the SNP variant at position 602 of the RHD coding sequence, it would not be possible to deduce a RHD*DIIIa haplotype for a sample, and therefore it would not be possible to predict a non-D⁻ phenotype for said sample.

Without the determination of the nucleotide at position 1048 of the RHD coding sequence, it would not be possible to deduce a RHD*DIVa-2 haplotype for a sample, and therefore it would not be possible to predict a non-D⁻ phenotype for said sample.

Without the determination of the presence/absence of RHD exon 3, it would not be possible to predict a RHD*DIIIa-CE(4-7)-D haplotype for samples with certain novel RHD variants, and therefore it would not be possible to predict a C^{+W} phenotype for those samples. For example, when at least one RHCE*C allele is absent, at least one RHD/RHCE hybrid exon 3 (RHD/CE Hex03) allele is present, the SNP at position 602 of the RHD coding sequence is absent, and the SNP at position 1048 of the RHD coding sequence is absent, the presence or absence of at least one RHD exon 3 allele will allow prediction of a C⁻ or a C^{+W} phenotype, respectively (Table 1). Similarly, when at least one RHCE*C allele is absent, at least one RHD/RHCE hybrid exon 3 (RHD/CE Hex03) allele is present, nucleotide sequence at position 602 of the RHD coding sequence is G, and nucleotide sequence at position 1048 of the RHD coding sequence is G, the absence or presence of at least one RHD exon 3 allele will allow prediction of a Partial D or an undetermined (Weak D or Partial D) phenotype, respectively.

A variety of suitable techniques could be used to detect these genetic sequences. The following are presented as non-limiting examples of such techniques.

A suitable technique to detect the herein mentioned genetic sequences is mutation analysis by restriction digestion after a PCR reaction for amplifying the region of interest, if the genetic variant or polymorphism results in the creation or elimination of a restriction site. Sequence analysis, such as direct manual or fluorescent automated sequencing, directly or after selection of the region of interest by PCR, can also be used to detect specific sequences. Allele-specific oligonucleotides, for example, used in a competitive PCR, can also be used to detect genetic variants.

Another technique to detect specific sequences in a sample is testing that sample for the presence of a nucleic acid molecule comprising all or a portion of the region of interest, comprising contacting said sample with a second nucleic acid molecule or probe under conditions for selective hybridization. All or a part of the region of interest can be amplified prior to performing the specific technique used for detection of the genetic variants.

In some embodiments, the invention comprises one or more of the specific PCR primers described herein. As understood in the art, the sequence of PCR primers may be altered without substantially modifying the ability of the primers to amplify nucleic acid. Therefore, in some embodiments the invention comprises variant PCR primers having nucleotide sequence alterations. Preferably, the variant has 1, 2, 3, 4, or 5 nucleotide sequence alterations compared to the PCR primer sequences described herein. A nucleotide sequence alteration may be an insertion, deletion or substitution.

The sample may be any biological sample from a patient, for example tissue, blood, serum or saliva from a patient. The sample may contain cells, be cell-free or consist only of isolated cells.

The methods of the invention make use of the detection of the presence or absence of one or more specific nucleotide sequences within the functional segments.

In certain cases, the method of the invention may be termed Allele-Specific Hybridization, and may make use of synthetic oligonucleotide probes usually 10-50 nucleotides long, preferably 19-27 nucleotides long, the sequences of which are designed to be complementary to the interrogated sequence. Complementarity of sequences enables pairing of genomic DNA and oligonucleotide probe molecules. Specific pairing, i.e. pairing of probes to their complementary sequence and to no other sequence, can be made to occur under appropriate conditions, which include but are not limited to the time of incubation, temperature of incubation, concentration of probe and complementary sequences, stringency of buffers, and mixing. Specific pairing to probes allows detection of sequences in a mix of sequences. Detection or lack of detection of specific sequences, in turn, allows determination of presence versus absence of functional segments.

Synthetic oligonucleotide probes can be used for the detection of particular conserved, non-variant regions and/or allelic variants in an individual's genomic DNA. Often, allelic variants are single nucleotide polymorphisms (SNPs), i.e. nucleotide positions at which the DNA composition may vary across individuals.

In some cases, the synthetic oligonucleotide probes described herein are designed and used to detect the presence or absence of functional nucleic acid segments and also, both to detect allelic variants located within sequences and to determine the presence or absence of functional segments.

Given a particular nucleotide at a particular position of a locus of genomic DNA, synthetic oligonucleotide molecules, or probes, can be designed to detect said nucleotide in a test sample. Probes can be designed in pairs such that one member of the probe pair is complementary to one strand of the sequence, whereas the other member of the probe pair is complementary to the other strand of the sequence. Probes can also be designed in sets so that they have different lengths and be complementary to one strand or the two strands of the sequence of interest.

In some embodiments, the invention comprises the specific oligonucleotide probes described herein. As understood in the art, the sequence of a probe may be altered without substantially modifying the ability of the probe to detect nucleic acid. Therefore, the invention further comprises variant probes having nucleotide sequence alterations. Preferably, the variant has 1, 2, 3, 4, or 5 nucleotide sequence alterations compared to the probes described herein. A nucleotide alteration may be an insertion, deletion or substitution.

In accordance with any aspect of the present invention, probes may be attached to a chemically-functionalized solid support. An example of a solid support is a flat glass surface, on which probe molecules are placed by contact deposition. Another example of a solid support is a particle such as a micrometer-size polymer bead, to which probe molecules are attached by conjugation. Another example of a solid support is a nanometer-size particle to which probe molecules are attached.

If the probes are immobilised on a flat glass surface, attachment of a probe to the surface may be performed at multiple individual locations, hereafter referred to as replicate features or “replicates”. The number of replicate features for each probe is usually ten, although it may vary. If the probes are immobilised on particles, attachment may be to multiple ensembles of particles.

In accordance with any aspect of the invention described herein, functional segments or their portions containing markers of the invention may be amplified, for example by PCR, using genomic DNA as a template. Amplified functional segments or their portions containing markers can be labeled (e.g. with a biotin and/or fluorescent label) to allow for their detection, and optionally fragmented to facilitate pairing with oligonucleotide probes.

In accordance with any aspect of the invention described herein, labelled and fragmented functional segments or their portions containing markers of the invention may be incubated under conditions that maximize the sensitivity and specificity of pairing with probes attached to the solid support. The presence of probe-paired functional segments or their portions may be determined indirectly from the measurement of a label, usually a fluorochrome, attached to the solid support. This measurement is referred to herein as signal intensity. By way of example, the fluorescence emitted by the fluorochrome may be collected by means of a fluorescence detection device, such as a confocal scanner.

### Determination of the Presence or Absence of RHCE*C

### Amplification of RHCE*C Intron 2 by PCR.

The marker RHCE*C may be detected by amplifying RHCE*C intron 2 using oligonucleotide primers that bind to intron 2 sequences. The target sequence of the forward (upstream) primer may be RHCE*C-specific, and the target sequence of the reverse (downstream) primer may be non-specific, i.e. shared by RHD, RHC*C, RHC*c. The following primers may be used, which yield a PCR product with a size of 357 base pairs:

(SEQ ID NO: 3)

| | |
|---|---|
| Forward primer: | 5′-GGCCACCACCATTTGAA-3′ |

(SEQ ID NO: 4)

| | |
|---|---|
| Reverse primer: | 5′-CCATGAACATGCCACTTCAC-3′ |

In boldface, RHCE*C-specific nucleotides forward
primer).

Alternatively, RHCE*C can be amplified using any oligonucleotide primers that differ from the previously described primers in sequence, length, or any other feature, as long as they enable specific amplification of the RHCE*C-specific insert located in intron 2. For example, a variant oligonucleotide primer may be used that differs from a primer described herein by 1, 2, 3, 4 or 5 nucleotide sequence alterations.

Alternatively, this step can make use of any oligonucleotide primers that enable the amplification of any other known RHCE*C-specific sequences, other than the previously described intron 2 insert, for the purpose of establishing the presence or absence of an RHCE*C allele in a sample. Such sequences usually contain polymorphic positions in the coding or non-coding regions of the RHCE gene. Instances include but are not limited to the following: position 48 (exon 1), position 150 (exon 2), position 178 (exon 2), position 201 (exon 2), position 203 (exon 2), and position 307 (exon 2) of the RHCE coding sequence.

The presence or absence of RHCE*C may then be visualized directly, for example by gel electrophoresis, or indirectly, for example by hybridization with a probe as discussed below. Alternatively, the presence or absence of RHCE*C may be determined by probe hybridization alone without prior PCR amplification.

Hybridization of RHCE*C to oligonucleotide probes.

In some embodiments, the hybridization of the RHCE*C intron 2 amplicon to oligonucleotide probes can make use of 4 oligonucleotide probes: 2 probes would be complementary to a portion of the RHCE*C-specific insert, each to one of the DNA strands. The other 2 probes would be identical to the above except that they contain an artificial single-nucleotide mismatch at their central position that largely prevents hybridization, thus providing a background signal as a negative control. These probes can only detect presence versus absence of the variant sequence, i.e. they do not allow discrimination between homozygous/hemizygous and heterozygous samples. The following probe sequences can be used for this insert:

RHCE*C-specific perfect match probe #1:
(SEQ ID NO: 5)
5′-TTTTACAGACGCCTGCTACCATG-3′
RHCE*C-specific perfect match probe #2:
(SEQ ID NO: 6)
5′-CATGGTAGCAGGCGTCTGTAAAA-3′
Mismatch probe #1:
(SEQ ID NO: 7)
5′-TTTTACAGACGTCTGCTACCATG-3′
Mismatch probe #2:
(SEQ ID NO: 8)
5′-CATGGTAGCAGACGTCTGTAAAA-3′
In boldface, the central position mismatch.

Alternatively, any particular method or set of probes targeting an amplified RHCE*C-specific amplicon, or an unamplified RHCE*C-specific sequence directly, may be used to determine whether RHCE*C is present or absent. Alternatively, a variant oligonucleotide probe may be used that differs from a probe described herein by 1, 2, 3, 4 or 5 nucleotide sequence alterations.

### Determination of the Presence or Absence of RHD/RHCE Hybrid Exon 3

### Amplification of RHD/RHCE Hybrid Exon 3 by PCR.

The marker RHD/RHCE hybrid exon 3 may be detected by PCR amplification using oligonucleotide primers that bind to intronic sequences flanking RHD/RHCE hybrid exon 3. Specifically, the target sequences of forward (upstream) and reverse (downstream) primers can be located in introns 2 and 3, respectively. The following primers may be used, which yield a PCR product of 256 base pairs:

(SEQ ID NO: 9)
Forward primer: 5′-TCCTGGCTCTCCCTCTCT-3′
(SEQ ID NO: 10)
Reverse primer: 5′-TTTTCAAAACCCCGGAAG-3′
In boldface, RHD-specific nucleotides (forward
primer) and RHCE-specific nucleotides (reverse
primer).

The forward primer may also be used for amplification of RHD exon 3, discussed below.

Alternatively, RHD/RHCE hybrid exon 3 may be amplified using any oligonucleotide primers that differ from the previously described primers in sequence, length, or any other feature, as long as they enable specific amplification of RHD/RHCE hybrid exon 3. For example, a variant oligonucleotide primer may be used that differs from a primer described herein by 1, 2, 3, 4 or 5 nucleotide sequence alterations.

Alternatively, PCR amplification can make use of any oligonucleotide primers that enable the amplification of any known RHD/RHCE hybrid exon 3-associated sequences for the purpose of establishing the presence or absence of an RHD/RHCE hybrid exon 3 in a sample. Such sequences usually contain polymorphic positions in the coding or non-coding regions of the RHD gene. Instances include but are not limited to position 178 (exon 2) of the RHD coding sequence.

The presence or absence of the RHD/RHCE hybrid exon 3 may then be visualized directly, for example by gel electrophoresis, or indirectly, for example by hybridization with a probe as discussed below. Alternatively, the presence or absence of the RHD/RHCE hybrid exon 3 may be determined by probe hybridization alone without prior PCR amplification.

Hybridization of RHD/RHCE Hybrid Exon 3 or RHD exon 3 Amplicon to Oligonucleotide Probes.

In some embodiments, the hybridization of the RHD/RHCE hybrid exon 3 amplicon to oligonucleotide probes can make use of 4 oligonucleotide probes. These probes may also be used to detect an SNP at RHD exon 3, as discussed below, due to the high similarity between these sequences, as specificity of the hybridization signal from each marker comes mainly from the allele specific PCR amplification step. For example, different fluorescently modified nucleotides may be incorporated during PCR amplification into the RHD/RHCE hybrid exon 3 and RHD exon 3 amplicons, respectively. Alternatively, the two amplicons may be differently labelled after the PCR amplification step, for example by incubation with labelled nucleotides or nucleotide oligos in the presence of a polymerase, ligase or transferase enzyme. For example, the nucleic acid may be incubated with biotinylated ddNTPs in the presence of a terminal deoxynucleotidyl transferase enzyme.

2 probes may be specific for the wild-type sequence of an SNP located within the amplicon, and the other 2 probes may be specific for the hybrid exon 3 variant sequence of the same SNP. These probes can only detect presence versus absence of the variant sequence, i.e. they do not allow discrimination between homozygous and hemizygous samples. For example, the SNP located at position 410 of the coding sequence, with C and T as wild-type and variant nucleotides, respectively, can be used. The following probe sequences can be used for this SNP:

(SEQ ID NO: 11)
RHD, RHCE wild-type probe #1:
5′-GGTCAACTTGGCGCAGTTGGTGG-3′
(SEQ ID NO: 12)
RHD, RHCE wild-type probe #2:
5′-GTCAACTTGGCGCAGTTGGTG-3′
(SEQ ID NO: 13)
Hybrid exon 3 variant probe #1:
5′-GGTCAACTTGGTGCAGTTGGTGG-3′
(SEQ ID NO: 14)
Hybrid exon 3 variant probe #2:
5′-GTCAACTTGGTGCAGTTGGTG-3′
In boldface is the SNP at position 410. The rs
number for the SNP at position 410 is not
available.

Alternatively, any particular method or set of probes targeting an amplified RHD/RHCE hybrid exon 3-specific amplicon, or an unamplified RHD/RHCE hybrid exon 3-specific sequence directly, may be used to determine whether RHD/RHCE hybrid exon 3 is present or absent. Alternatively, a variant oligonucleotide probe may be used that differs from a probe described herein by 1, 2, 3, 4 or 5 nucleotide sequence alterations.

### Determination of the Presence or Absence of RHD Exon 3

### Amplification of RHD Exon 3 by PCR.

The marker RHD exon 3 may be detected by PCR amplification using oligonucleotide primers that bind to intronic sequences flanking RHD exon 3. Specifically, the target sequences of forward and reverse primers may be located in introns 2 and 3, respectively. The following primers may be used, which yield a PCR product of 268 base pairs:

(SEQ ID NO: 15)
Forward primer: 5′-TCCTGGCTCTCCCTCTCT-3′
(SEQ ID NO: 16)
Reverse primer: 5′-GTTGTCTTTATTTTTCAAAACCCT-3′
In boldface are the RHD-specific nucleotides. The
forward primer is specific for both RHD exon 3 and
RHD/RHCE hybrid exon 3, while the reverse primer
is specific for RHD exon 3 only.

Alternatively, RHD exon 3 may be amplified using any oligonucleotide primers that differ from the previously described primers in sequence, length, or any other feature, as long as they enable specific amplification of RHD exon 3.

Alternatively, PCR amplification can make use of any oligonucleotide primers that enable the amplification of publicly-reported RHD exon 3-associated sequences for the purpose of establishing the presence or absence of an RHD exon 3 in a sample. Such sequences usually contain polymorphic positions in the coding or non-coding regions of the RHD gene. Instances include but are not limited to intron sequences flanking RHD exon 3. For example, a variant oligonucleotide primer may be used that differs from a primer described herein by 1, 2, 3, 4 or 5 nucleotide sequence alterations.

The presence or absence of the RHD exon 3 may then be visualized directly, for example by gel electrophoresis, or indirectly, for example by hybridization with a probe as discussed with reference to RHD/RHCE hybrid exon 3 above. Alternatively, the presence or absence of the RHD exon 3 may be determined by probe hybridization alone without prior PCR amplification.

### Determination of the SNPs at RHD Exon 4 or RHD Exon 5 or RHD Exon 6

### Amplification of RHD Exon 4 or RHD Exon 5 or RHD Exon 6 by PCR.

In some embodiments, the SNP at RHD exon 4 may be detected by PCR amplification using oligonucleotide primers that bind to intronic sequences flanking RHD exon 4. Specifically, the target sequences of forward and reverse primers may be located in introns 3 and 4, respectively. The following primers may be used, which yield a PCR product of 281 base pairs:

| | |
|---|---|
| Forward primer: | |
| 5′-GCTCTGAACTTTCTCCAAGGACT-3′ | (SEQ ID NO: 17) |
| Reverse primer: | |
| 5′-ATTCTGCTCAGCCCAAGTAG-3′ | (SEQ ID NO: 18) |

In boldface are RHD-specific nucleotides.

In another embodiment, the SNP at RHD exon 5 may be detected by PCR amplification using oligonucleotide primers that bind to intronic sequences flanking RHD exon 5. Specifically, the target sequences of forward and reverse primers can be located in introns 4 and 5, respectively. The following primers may be used, which yield a PCR product of 432 base pairs:

(SEQ ID NO: 19)
Forward primer: 5′-TTGAATTAAGCACTTCACAGAGCA-3′
(SEQ ID NO: 20)
Reverse primer: 5′-CACCTTGCTGATCTTCCC-3′
The underline indicates the location of the RHCE-
specific 653-base pair insert exploited to confer
RHD specificity to the amplification. In boldface,
RHD-specific nucleotides.

In yet another embodiment, the SNP at RHD exon 6 may be detected by PCR amplification using oligonucleotide primers that bind to intronic sequences flanking RHD exon 6. Specifically, the target sequences of forward and reverse primers can be located in introns 5 and 6, respectively. The following primers may be used, which yield a PCR product of 371 base pairs:

(SEQ ID NO: 21)
Forward primer: 5′-AGTAGTGAGCTGGCCCATCA-3′
(SEQ ID NO: 22)
Reverse primer: 5′-CTTCAGCCAAAGCAGAGGAG-3′
In boldface, RHD-specific nucleotides.

The presence or absence of these SNPs, and the specific SNP variant, may then be visualized directly, for example by gel electrophoresis after restriction digest as described above, or indirectly, for example by hybridization with a probe as discussed below. Alternatively, the SNP variant may be determined by probe hybridization alone without prior PCR amplification.

A variant oligonucleotide primer may be used that differs from a primer described herein by 1, 2, 3, 4 or 5 nucleotide sequence alterations.

Hybridization of RHD exon 4 Amplicon or RHD exon 5 Amplicon or RHD Exon 6 Amplicon to Oligonucleotide Probes

In some embodiments, the hybridization of the RHD exon 4 amplicon to oligonucleotide probes can make use of 4 oligonucleotide probes: 2 probes are specific for the wild-type sequence of an SNP linked to RHD variants and located within the RHD exon 4 amplicon. The other 2 probes enable the RHD*DIIIa and RHD*DIIIa-CE(4-7)-D variants to be distinguished from the RHD*DIVa-2 variant, as they are specific for the RHD*DIIIa and RHD*DIIIa-CE(4-7)-D variants relative to RHD*DIVa-2. However, in some embodiments the probes may not distinguish between other RHD variants. In other words, that the individual SNPs by themselves may not uniquely identify a particular RHD variant (for example, due to the existence of other variants RHD variants, in addition to DIIIa, DIVa and RHD*DIIIa-CE(4-7)-D), but this SNP, and its probes, can distinguish one of the three variants DIIIa, DIVa and RHD*DIIIa-CE(4-7)-D from the other two.

These probes allow discrimination between homozygous/hemizygous and heterozygous samples. For example, the SNP located at position 602 of the coding sequence, with C and G as wild-type and RHD variant nucleotides, respectively, can be used. The following probe sequences can be used for this SNP:

(SEQ ID NO: 23)
RHD wild-type probe #1:
5′-ATAAAGATCAGACAGCAACGATACC-3′
(SEQ ID NO: 24)
RHD wild-type probe #2:
5′-TAAAGATCAGACAGCAACGATAC-3′
(SEQ ID NO: 25)
RHD variant probe #1:
5′-ATAAAGATCAGAGAGCAACGATACC-3′
(SEQ ID NO: 26)
RHD variant probe #2:
5′-TAAAGATCAGAGAGCAACGATAC-3′
In boldface is the SNP.

In another embodiment, the hybridization of the RHD exon 5 amplicon to oligonucleotide probes can make use of 4 oligonucleotide probes: 2 probes are specific for the wild-type sequence (i.e. sequence identical to the conventional RHD*D allele) of an SNP linked to RHD variants and located within the RHD exon 5 amplicon. The other 2 probes enable the RHD*DIIIa and RHD*DIIIa-CE(4-7)-D variants to be distinguished from the RHD*DIVa-2 variant, as they are specific for the RHD*DIIIa and RHD*DIIIa-CE(4-7)-D variants relative to RHD*DIVa-2. However, in some embodiments the probes may not distinguish between other RHD variants. These probes allow discrimination between homozygous/hemizygous and heterozygous samples. For example, the SNP located at position 667 of the coding sequence, with T and G as wild-type and RHD variant nucleotides, respectively, can be used. The following probe sequences can be used for this SNP:

(SEQ ID NO: 27)
RHD wild-type probe #1:
5′-CTGGCCAAGTTTCAACTCTGC-3′
(SEQ ID NO: 28)
RHD wild-type probe #2:
5′-TGGCCAAGTTTCAACTCTG-3′
(SEQ ID NO: 29)
RHD variant probe #1:
5′-CTGGCCAAGTGTCAACTCTGC-3′
(SEQ ID NO: 30)
RHD variant probe #2:
5′-TGGCCAAGTGTCAACTCTG-3′
In boldface is the SNP.

In yet another embodiment, the hybridization of the RHD exon 6 amplicon to oligonucleotide probes can make use of 4 oligonucleotide probes: 2 probes would be specific for the wild-type sequence (i.e. sequence identical to the conventional RHD*D allele) of a SNP located within the RHD exon 6 amplicon, and linked to the RHD*DIIIa variant. The other 2 probes enable the RHD*DIIIa variant to be distinguished from RHD*DIIIa-CE(4-7)-D and RHD*DIVa-2, as they are completely specific for the RHD*DIIIa variant relative to RHD*DIIIa-CE(4-7)-D and RHD*DIVa-2. However, the probes may only be partially specific for the RHD*DIIIa variant sequence of the same SNP versus other RHD variants (i.e. RHD variants other than DIIIa, DIVa and RHD*DIIIa-CE(4-7)-D). These probes allow discrimination between homozygous/hemizygous and heterozygous samples. For example, the SNP located at position 819 of the coding sequence, with G and A as wild-type and RHD*DIIIa variant nucleotides, respectively, can be used. The following probe sequences can be used for this SNP:

(SEQ ID NO: 31)
RHD wild-type probe #1:
5′-GTGCACAGTGCGGTGTTGGCAGG-3′
(SEQ ID NO: 32)
RHD wild-type probe #2:
5′-TGCACAGTGCGGTGTTGGCAG-3′
(SEQ ID NO: 33)
RHD*DIIIα variant probe #1:
5′-GTGCACAGTGCAGTGTTGGCAGG -3′
(SEQ ID NO: 34)
RHD*DIIIα variant probe #2:
5′-TGCACAGTGCAGTGTTGGCAG-3′
In boldface is the SNP. The rs number of the SNP
at position 819 is not available.

Alternatively, a variant oligonucleotide probe may be used that differs from a probe described herein by 1, 2, 3, 4 or 5 nucleotide sequence alterations.

### Determination of the SNP at RHD Exon 7

### Amplification of RHD Exon 7 by PCR

The SNP variant at RHD exon 7 may be amplified using oligonucleotide primers that bind to intronic sequences flanking RHD exon 7. Specifically, the target sequences of forward and reverse primers may be located in introns 6 and 7, respectively. The following primers may be used, which yield a PCR product of 695 base pairs:

(SEQ ID NO: 35)
Forward primer: 5′-ACAAACTCCCCGATGATGTGAGTG-3′
(SEQ ID NO: 36)
Reverse primer: 5′-GAGGCTGAGAAAGGTTAAGCCA-3′
In boldface are RHD-specific nucleotides.

The presence or absence of this SNP, and the specific SNP variant, may then be visualized directly, for example by gel electrophoresis after restriction digest as described above, or indirectly, for example by hybridization with a probe as discussed below. Alternatively, the SNP variant may be determined by probe hybridization alone without prior PCR amplification.

A variant oligonucleotide primer may be used that differs from a primer described herein by 1, 2, 3, 4 or 5 nucleotide sequence alterations.

### Hybridization of RHD Exon 7 Amplicon to Oligonucleotide Probes.

The hybridization of the RHD exon 7 amplicon to oligonucleotide probes can make use of 4 oligonucleotide probes: 2 probes would be specific for the wild-type sequence (i.e. sequence identical to the conventional RHD*D allele) of a SNP located within the amplicon, and linked to RHD variants. The other 2 probes enable the RHD*DIVa-2IIIa variant to be distinguished from RHD*DIIIa-CE(4-7)-D and RHD*DIIIa, as the presence of the SNP is characteristic for the RHD*DIVa-2 variant. However, in some embodiments the probes may not distinguish between other RHD variants (i.e. RHD variants other than DIIIa, DIVa and RHD*DIIIa-CE(4-7)-D). These probes allow discrimination between homozygous/hemizygous and heterozygous samples. For example, the SNP located at position 1048 of the coding sequence, with G and C as wild-type and RHD*DIVa-2 variant nucleotides, respectively, can be used. The following probe sequences can be used for this SNP:

(SEQ ID NO: 37)
RHD wild-type probe #1:
5′-TGCTGGTGCTTGATACCGTCGGA-3′
(SEQ ID NO: 38)
RHD wild-type probe #2:
5′-GCTGGTGCTTGATACCGTCGG-3′
(SEQ ID NO: 39)
RHD*DIVα-2 variant probe #1:
5′-TGCTGGTGCTTCATACCGTCGGA-3′
(SEQ ID NO: 40)
RHD*DIVα-2 variant probe #2:
5′-GCTGGTGCTTCATACCGTCGG-3′
In boldface is the SNP.

Alternatively, a variant oligonucleotide probe may be used that differs from a probe described herein by 1, 2, 3, 4 or 5 nucleotide sequence alterations.

Determination of binding of amplified sequences to wild-type versus variant probes is typically, but not solely, performed through quantitation of probe-bound fluorescence. Fluorescence and/or fluorescence-capturing moieties are typically, but not solely, introduced into the process at the amplification step in the form of modified nucleotides (see Materials & Methods section).

TABLE 1

| RHCE*C | RHD/CE Hex03 | RHD ex 03 | RHD 602 | RHD 1048 | RHD Haplotype 1/RHD Haplotype 2 | RHD, RHC Phenotype |
|---|---|---|---|---|---|---|
| Absent | Present | Present | C | G | RHD*DIIIa-CE(4-7)-D/Possibly RHD | D?, C^{+W} |
| Absent | Present | Present | C | G/C | RHD*DIVa-2/Possibly RHD | D?, C⁻ |
| Absent | Present | Present | C | C | RHD*DIVa-2/RHD*DIVb-4 | Partial D, C⁻ |
| Absent | Present | Present | C | Absent | Not Reported | |
| Absent | Present | Present | C/G | G | RHD*DIIIa/Possibly RHD | D?, C⁻ |
| Absent | Present | Present | C/G | G/C | RHD*DIIIa/RHD*DIVb-4 | Partial D, C⁻ |
| | | | | | RHD*weakDtype4.0/RHD*DIVa-2 | Weak or Partial D, C⁻ |
| | | | | | RHD*weakDtype4.1/RHD*DIVa-2 | Weak or Partial D, C⁻ |
| | | | | | RHD*weakDtype14/RHD*DIVa-2 | Weak or Partial D, C⁻ |
| | | | | | RHD*weakDtype51/RHD*DIVa-2 | Weak or Partial D, C⁻ |
| | | | | | RHD*DAR/RHD*DIVa-2 | Partial D, C⁻ |
| | | | | | RHD*DAR-E/RHD*DIVa-2 | Partial D, C⁻ |
| Absent | Present | Present | C/G | C | Not Reported | |
| Absent | Present | Present | C/G | Absent | Not Reported | |
| Absent | Present | Present | G | G | RHD*weakDtype4.0/RHD*DIIIa | Weak or Partial D, C |
| | | | | | RHD*weakDtype4.1/RHD*DIIIa | Weak or Partial D, C⁻ |
| | | | | | RHD*weakDtype14/RHD*DIIIa | Weak or Partial D, C⁻ |
| | | | | | RHD*weakDtype51/RHD*DIIIa | Weak or Partial D, C⁻ |
| | | | | | RHD*DAR/RHD*DIIIa | Partial D?, C⁻ |
| | | | | | RHD*DAR-E/RHD*DIIIa | Partial D?, C⁻ |
| | | | | | RHD*weakDtype4.0/RHD*DIIIa-CE(4-7)-D) | Weak D, C^{+W} |
| | | | | | RHD*weakDtype4.1/RHD*DIIIa-CE(4-7)-D) | Weak D, C^{+W} |
| | | | | | RHD*weakDtype14/RHD*DIIIa-CE(4-7)-D) | Weak D, C^{+W} |
| | | | | | RHD*weakDtype51/RHD*DIIIa-CE(4-7)-D) | Weak D, C^{+W} |
| | | | | | RHD*DAR/RHD*DIIIa-CE(4-7)-D) | Partial D, C^{+W} |
| | | | | | RHD*DAR-E/RHD*DIIIa-CE(4-7)-D) | Partial D, C^{+W} |
| Absent | Present | Present | G | G/C | Not Reported | |
| Absent | Present | Present | G | C | Not Reported | |
| Absent | Present | Present | G | Absent | Not Reported | |
| Absent | Present | Present | Absent | G | Not Reported | |
| Absent | Present | Present | Absent | G/C | Not Reported | |
| Absent | Present | Present | Absent | C | Not Reported | |
| Absent | Present | Present | Absent | Absent | RHD*ex04-ex07del | D⁻, C⁻ |
| Absent | Present | Absent | C | G | RHD*DIIIa-CE(4-7)-D/RHD*ex03del | D⁻, C^{+W} |
| Absent | Present | Absent | C | G/C | RHD*DIVa-2/RHD*ex03del | Partial D, C⁻ |
| Absent | Present | Absent | C | C | RHD*DIVa-2/No RHD | Partial D, C⁻ |
| | | | | | RHD*DIVa-2/RHD*DIVa-2 | Partial D, C⁻ |
| | | | | | RHD*DIVa-2/RHD*DIIIa-CE(4-7)-D | Partial D, C^{+W} |
| Absent | Present | Absent | C | Absent | Not Reported | |
| Absent | Present | Absent | C/G | G | RHD*DIIIa/RHD*ex03del | Partial D, C⁻ |
| Absent | Present | Absent | C/G | G/C | RHD*DIIIa/RHD*DIVa-2 | Partial D, C⁻ |
| Absent | Present | Absent | C/G | C | Not Reported | |
| Absent | Present | Absent | C/G | Absent | Not Reported | |
| Absent | Present | Absent | G | G | RHD*DIIIa/No RHD | Partial D, C⁻ |
| | | | | | RHD*DIIIa/RHD*DIIIa | Partial D, C⁻ |
| | | | | | RHD*DIIIa/RHD*DIIIa-CE(4-7)-D | Partial D, C^{+W} |
| Absent | Present | Absent | G | G/C | RHD*DIIIa/RHD*ex03-ex04del | Partial D, C⁻ |
| Absent | Present | Absent | G | C | Not Reported | |
| Absent | Present | Absent | G | Absent | Not Reported | |
| Absent | Present | Absent | Absent | G | RHD*DIIIa-CE(4-7)-D/RHD*ex03-ex04del | D⁻, C^{+W} |
| Absent | Present | Absent | Absent | G/C | Not Reported | |
| Absent | Present | Absent | Absent | C | Not Reported | |
| Absent | Present | Absent | Absent | Absent | RHD*DIIIa-CE(4-7)-D/No RHD | D⁻, C^{+W} |
| | | | | | RHD*DIIIa-CE(4-7)-D/RHD*DIIIa-CE(4-7)-D | D⁻, C^{+W} |
| Absent | Absent | Present | C | G | Possibly RHD | D?, C⁻ |
| Absent | Absent | Present | C | G/C | RHD*DIVb-4/Possibly RHD | D?, C⁻ |
| Absent | Absent | Present | C | C | RHD*DIVb-4/RHD*DIVb-4 | Partial D, C⁻ |
| Absent | Absent | Present | C | Absent | Not Reported | |
| Absent | Absent | Present | C/G | G | RHD*weakDtype4.0/Possibly RHD | D?, C⁻ |
| | | | | | RHD*weakDtype4.1/Possibly RHD | D?, C⁻ |
| | | | | | RHD*weakDtype14/Possibly RHD | D?, C⁻ |
| | | | | | RHD*weakDtype51/Possibly RHD | D?, C⁻ |
| | | | | | RHD*DAR/Possibly RHD | D?, C⁻ |
| | | | | | RHD*DAR-E/Possibly RHD | D?, C⁻ |
| Absent | Absent | Present | C/G | G/C | RHD*weakDtype4.0/RHD*DIVb-4 | Weak or Partial D, C⁻ |
| | | | | | RHD*weakDtype4.0/RHD*DIVb-4 | Weak or Partial D, C⁻ |
| | | | | | RHD*weakDtype4.1/RHD*DIVb-4 | Weak or Partial D, C⁻ |
| | | | | | RHD*weakDtype14/RHD*DIVb-4 | Weak or Partial D, C⁻ |
| | | | | | RHD*weakDtype51/RHD*DIVb-4 | Weak or Partial D, C⁻ |
| | | | | | RHD*DAR/RHD*DIVb-4 | Partial D, C⁻ |
| | | | | | RHD*DAR-E/RHD*DIVb-4 | Partial D, C⁻ |
| Absent | Absent | Present | C/G | C | Not Reported | |
| Absent | Absent | Present | C/G | Absent | Not Reported | |
| Absent | Absent | Present | G | G | RHD*weakDtype4.0/RHD*weakDtype4.0 | Weak D, C⁻ |
| | | | | | RHD*weakDtype4.1/RHD*weakDtype4.0 | Weak D, C⁻ |
| | | | | | RHD*weakDtype14/RHD*weakDtype4.0 | Weak D, C⁻ |
| | | | | | RHD*weakDtype51/RHD*weakDtype4.0 | Weak D, C⁻ |
| | | | | | RHD*DAR/RHD*weakDtype4.0 | Weak or Partial D, C⁻ |
| | | | | | RHD*DAR-E/RHD*weakDtype4.0 | Weak or Partial D, C⁻ |
| | | | | | RHD*weakDtype4.0/RHD*weakDtype4.1 | Weak D, C⁻ |
| | | | | | RHD*weakDtype4.1/RHD*weakDtype4.1 | Weak D, C⁻ |
| | | | | | RHD*weakDtype14/RHD*weakDtype4.1 | Weak D, C⁻ |
| | | | | | RHD*weakDtype51/RHD*weakDtype4.1 | Weak D, C⁻ |
| | | | | | RHD*DAR/RHD*weakDtype4.1 | Weak or Partial D, C⁻ |
| | | | | | RHD*DAR-E/RHD*weakDtype4.1 | Weak or Partial D, C⁻ |
| | | | | | RHD*weakDtype4.0/RHD*weakDtype14 | Weak D, C⁻ |
| | | | | | RHD*weakDtype4.1/RHD*weakDtype14 | Weak D, C⁻ |
| | | | | | RHD*weakDtype14/RHD*weakDtype14 | Weak D, C⁻ |
| | | | | | RHD*weakDtype51/RHD*weakDtype14 | Weak D, C⁻ |
| | | | | | RHD*DAR/RHD*weakDtype14 | Weak or Partial D, C⁻ |
| | | | | | RHD*DAR-E/RHD*weakDtype14 | Weak or Partial D, C⁻ |
| | | | | | RHD*weakDtype4.0/RHD*weakDtype51 | Weak D, C⁻ |
| | | | | | RHD*weakDtype4.1/RHD*weakDtype51 | Weak D, C⁻ |
| | | | | | RHD*weakDtype14/RHD*weakDtype51 | Weak D, C⁻ |
| | | | | | RHD*weakDtype51/RHD*weakDtype51 | Weak D, C⁻ |
| | | | | | RHD*DAR/RHD*weakDtype51 | Weak or Partial D, C⁻ |
| | | | | | RHD*DAR-E/RHD*weakDtype51 | Weak or Partial D, C⁻ |
| | | | | | RHD*weakDtype4.0/RHD*DAR | Weak or Partial D, C⁻ |
| | | | | | RHD*weakDtype4.1/RHD*DAR | Weak or Partial D, C⁻ |
| | | | | | RHD*weakDtype14/RHD*DAR | Weak or Partial D, C⁻ |
| | | | | | RHD*weakDtype51/RHD*DAR | Weak or Partial D, C⁻ |
| | | | | | RHD*DAR/RHD*DAR | Partial D, C⁻ |
| | | | | | RHD*DAR-E/RHD*DAR | Partial D, C⁻ |
| | | | | | RHD*weakDtype4.0/RHD*DAR-E | Weak or Partial D, C⁻ |
| | | | | | RHD*weakDtype4.1/RHD*DAR-E | Weak or Partial D, C⁻ |
| | | | | | RHD*weakDtype14/RHD*DAR-E | Weak or Partial D, C⁻ |
| | | | | | RHD*weakDtype51/RHD*DAR-E | Weak or Partial D, C⁻ |
| | | | | | RHD*DAR/RHD*DAR-E | Partial D, C⁻ |
| | | | | | RHD*DAR-E/RHD*DAR-E | Partial D, C⁻ |
| Absent | Absent | Present | G | G/C | Not Reported | |
| Absent | Absent | Present | G | C | Not Reported | |
| Absent | Absent | Present | G | Absent | Not Reported | |
| Absent | Absent | Present | Absent | G | Not Reported | |
| Absent | Absent | Present | Absent | G/C | Not Reported | |
| Absent | Absent | Present | Absent | C | Not Reported | |
| Absent | Absent | Present | Absent | Absent | RHD*ex04-ex07del/RHD*ex04-ex07del | D⁻, C⁻ |
| | | | | | RHD*ex04-ex07del/No RHD | D⁻, C⁻ |
| Absent | Absent | Absent | C | G | RHD*ex03del/RHD*ex03del | D⁻, C⁻ |
| | | | | | RHD*ex03del/No RHD | D⁻, C⁻ |
| Absent | Absent | Absent | C | G/C | Not Reported | |
| Absent | Absent | Absent | C | C | Not Reported | |
| Absent | Absent | Absent | C | Absent | Not Reported | |
| Absent | Absent | Absent | C/G | G | Not Reported | |
| Absent | Absent | Absent | C/G | G/C | Not Reported | |
| Absent | Absent | Absent | C/G | C | Not Reported | |
| Absent | Absent | Absent | C/G | Absent | Not Reported | |
| Absent | Absent | Absent | G | G | Not Reported | |
| Absent | Absent | Absent | G | G/C | Not Reported | |
| Absent | Absent | Absent | G | C | Not Reported | |
| Absent | Absent | Absent | G | Absent | Not Reported | |
| Absent | Absent | Absent | Absent | G | RHD*ex03-ex04del/RHD*ex03-ex04del | D⁻, C⁻ |
| | | | | | RHD*ex03-ex04del/No RHD | D⁻, C⁻ |
| Absent | Absent | Absent | Absent | G/C | Not Reported | |
| Absent | Absent | Absent | Absent | C | Not Reported | |
| Absent | Absent | Absent | Absent | Absent | No RHD/No RHD | D⁻, C⁻ |
| Present | Present | Present | C | G | RHD*DIIIa-CE(4-7)-D/Possibly RHD | D?, C⁺ |
| Present | Present | Present | C | G/C | RHD*DIVa-2/Possibly RHD | D?, C⁺ |
| Present | Present | Present | C | C | RHD*DIVa-2/RHD*DIVb-4 | Partial D, C⁺ |
| Present | Present | Present | C | Absent | Not Reported | |
| Present | Present | Present | C/G | G | RHD*DIIIa/Possibly RHD | D?, C⁺ |
| Present | Present | Present | C/G | G/C | RHD*DIIIa/RHD*DIVb-4 | Partial D, C⁺ |
| | | | | | RHD*weakDtype4.0/RHD*DIVa-2 | Weak or Partial D, C⁺ |
| | | | | | RHD*weakDtype4.1/RHD*DIVa-2 | Weak or Partial D, C⁺ |
| | | | | | RHD*weakDtype14/RHD*DIVa-2 | Weak or Partial D, C⁺ |
| | | | | | RHD*weakDtype51/RHD*DIVa-2 | Weak or Partial D, C⁺ |
| | | | | | RHD*DAR/RHD*DIVa-2 | Partial D, C⁺ |
| | | | | | RHD*DAR-E/RHD*DIVa-2 | Partial D, C⁺ |
| Present | Present | Present | C/G | C | Not Reported | |
| Present | Present | Present | C/G | Absent | Not Reported | |
| Present | Present | Present | G | G | RHD*weakDtype4.0/RHD*DIIIa | Weak or Partial D, C⁺ |
| | | | | | RHD*weakDtype4.1/RHD*DIIIa | Weak or Partial D, C⁺ |
| | | | | | RHD*weakDtype14/RHD*DIIIa | Weak or Partial D, C⁺ |
| | | | | | RHD*weakDtype51/RHD*DIIIa | Weak or Partial D, C⁺ |
| | | | | | RHD*DAR/RHD*DIIIa | Partial D?, C⁺ |
| | | | | | RHD*DAR-E/RHD*DIIIa | Partial D?, C⁺ |
| | | | | | RHD*weakDtype4.0/RHD*DIIIa-CE(4-7)-D) | Weak D, C⁺ |
| | | | | | RHD*weakDtype4.1/RHD*DIIIa-CE(4-7)-D) | Weak D, C⁺ |
| | | | | | RHD*weakDtype14/RHD*DIIIa-CE(4-7)-D) | Weak D, C⁺ |
| | | | | | RHD*weakDtype51/RHD*DIIIa-CE(4-7)-D) | Weak D, C⁺ |
| | | | | | RHD*DAR/RHD*DIIIa-CE(4-7)-D) | Partial D, C⁺ |
| | | | | | RHD*DAR-E/RHD*DIIIa-CE(4-7)-D) | Partial D, C⁺ |
| Present | Present | Present | G | G/C | Not Reported | |
| Present | Present | Present | G | C | Not Reported | |
| Present | Present | Present | G | Absent | Not Reported | |
| Present | Present | Present | Absent | G | Not Reported | |
| Present | Present | Present | Absent | G/C | Not Reported | |
| Present | Present | Present | Absent | C | Not Reported | |
| Present | Present | Present | Absent | Absent | RHD*ex04-ex07del | D⁻, C⁺ |
| Present | Present | Absent | C | G | RHD*DIIIa-CE(4-7)-D/RHD*ex03del | D⁻, C⁺ |
| Present | Present | Absent | C | G/C | RHD*DIVa-2/RHD*ex03del | Partial D, C⁺ |
| Present | Present | Absent | C | C | RHD*DIVa-2/No RHD | Partial D, C⁺ |
| | | | | | RHD*DIVa-2/RHD*DIVa-2 | Partial D, C⁺ |
| | | | | | RHD*DIVa-2/RHD*DIIIa-CE(4-7)-D | Partial D, C⁺ |
| Present | Present | Absent | C | Absent | Not Reported | |
| Present | Present | Absent | C/G | G | RHD*DIIIa/RHD*ex03del | Partial D, C⁺ |
| Present | Present | Absent | C/G | G/C | RHD*DIIIa/RHD*DIVa-2 | Partial D, C⁺ |
| Present | Present | Absent | C/G | C | Not Reported | |
| Present | Present | Absent | C/G | Absent | Not Reported | |
| Present | Present | Absent | G | G | RHD*DIIIa/No RHD | Partial D, C⁺ |
| | | | | | RHD*DIIIa/RHD*DIIIa | Partial D, C⁺ |
| | | | | | RHD*DIIIa/RHD*DIIIa-CE(4-7)-D | Partial D, C⁺ |
| Present | Present | Absent | G | G/C | RHD*DIIIa/RHD*ex03-ex04del | Partial D, C⁺ |
| Present | Present | Absent | G | C | Not Reported | |
| Present | Present | Absent | G | Absent | Not Reported | |
| Present | Present | Absent | Absent | G | RHD*DIIIa-CE(4-7)-D/RHD*ex03-ex04del | D⁻, C⁺ |
| Present | Present | Absent | Absent | G/C | Not Reported | |
| Present | Present | Absent | Absent | C | Not Reported | |
| Present | Present | Absent | Absent | Absent | RHD*DIIIa-CE(4-7)-D/No RHD | D⁻, C⁺ |
| | | | | | RHD*DIIIa-CE(4-7)-D/RHD*DIIIa-CE(4-7)-D | D⁻, C⁺ |
| Present | Absent | Present | C | G | Possibly RHD | D?, C⁺ |
| Present | Absent | Present | C | G/C | RHD*DIVb-4/Possibly RHD | D?, C⁺ |
| Present | Absent | Present | C | C | RHD*DIVb-4/RHD*DIVb-4 | Partial D, C⁺ |
| Present | Absent | Present | C | Absent | Not Reported | |
| Present | Absent | Present | C/G | G | RHD*weakDtype4.0/Possibly RHD | D?, C⁺ |
| | | | | | RHD*weakDtype4.1/Possibly RHD | D?, C⁺ |
| | | | | | RHD*weakDtype14/Possibly RHD | D?, C⁺ |
| | | | | | RHD*weakDtype51/Possibly RHD | D?, C⁺ |
| | | | | | RHD*DAR/Possibly RHD | D?, C⁺ |
| | | | | | RHD*DAR-E/Possibly RHD | D?, C⁺ |
| Present | Absent | Present | C/G | G/C | RHD*weakDtype4.0/RHD*DIVb-4 | Weak or Partial D, C⁺ |
| | | | | | RHD*weakDtype4.0/RHD*DIVb-4 | Weak or Partial D, C⁺ |
| | | | | | RHD*weakDtype4.1/RHD*DIVb-4 | Weak or Partial D, C⁺ |
| | | | | | RHD*weakDtype14/RHD*DIVb-4 | Weak or Partial D, C⁺ |
| | | | | | RHD*weakDtype51/RHD*DIVb-4 | Weak or Partial D, C⁺ |
| | | | | | RHD*DAR/RHD*DIVb-4 | Partial D, C⁺ |
| | | | | | RHD*DAR-E/RHD*DIVb-4 | Partial D, C⁺ |
| Present | Absent | Present | C/G | C | Not Reported | |
| Present | Absent | Present | C/G | Absent | Not Reported | |
| Present | Absent | Present | G | G | RHD*weakDtype4.0/RHD*weakDtype4.0 | Weak D, C⁺ |
| | | | | | RHD*weakDtype4.1/RHD*weakDtype4.0 | Weak D, C⁺ |
| | | | | | RHD*weakDtype14/RHD*weakDtype4.0 | Weak D, C⁺ |
| | | | | | RHD*weakDtype51/RHD*weakDtype4.0 | Weak D, C⁺ |
| | | | | | RHD*DAR/RHD*weakDtype4.0 | Weak or Partial D, C⁺ |
| | | | | | RHD*DAR-E/RHD*weakDtype4.0 | Weak or Partial D, C⁺ |
| | | | | | RHD*weakDtype4.0/RHD*weakDtype4.1 | Weak D, C⁺ |
| | | | | | RHD*weakDtype4.1/RHD*weakDtype4.1 | Weak D, C⁺ |
| | | | | | RHD*weakDtype14/RHD*weakDtype4.1 | Weak D, C⁺ |
| | | | | | RHD*weakDtype51/RHD*weakDtype4.1 | Weak D, C⁺ |
| | | | | | RHD*DAR/RHD*weakDtype4.1 | Weak or Partial D, C⁺ |
| | | | | | RHD*DAR-E/RHD*weakDtype4.1 | Weak or Partial D, C⁺ |
| | | | | | RHD*weakDtype4.0/RHD*weakDtype14 | Weak D, C⁺ |
| | | | | | RHD*weakDtype4.1/RHD*weakDtype14 | Weak D, C⁺ |
| | | | | | RHD*weakDtype14/RHD*weakDtype14 | Weak D, C⁺ |
| | | | | | RHD*weakDtype51/RHD*weakDtype14 | Weak D, C⁺ |
| | | | | | RHD*DAR/RHD*weakDtype14 | Weak or Partial D, C⁺ |
| | | | | | RHD*DAR-E/RHD*weakDtype14 | Weak or Partial D, C⁺ |
| | | | | | RHD*weakDtype4.0/RHD*weakDtype51 | Weak D, C⁺ |
| | | | | | RHD*weakDtype4.1/RHD*weakDtype51 | Weak D, C⁺ |
| | | | | | RHD*weakDtype14/RHD*weakDtype51 | Weak D, C⁺ |
| | | | | | RHD*weakDtype51/RHD*weakDtype51 | Weak D, C⁺ |
| | | | | | RHD*DAR/RHD*weakDtype51 | Weak or Partial D, C⁺ |
| | | | | | RHD*DAR-E/RHD*weakDtype51 | Weak or Partial D, C⁺ |
| | | | | | RHD*weakDtype4.0/RHD*DAR | Weak or Partial D, C⁺ |
| | | | | | RHD*weakDtype4.1/RHD*DAR | Weak or Partial D, C⁺ |
| | | | | | RHD*weakDtype14/RHD*DAR | Weak or Partial D, C⁺ |
| | | | | | RHD*weakDtype51/RHD*DAR | Weak or Partial D, C⁺ |
| | | | | | RHD*DAR/RHD*DAR | Partial D, C⁺ |
| | | | | | RHD*DAR-E/RHD*DAR | Partial D, C⁺ |
| | | | | | RHD*weakDtype4.0/RHD*DAR-E | Weak or Partial D, C⁺ |
| | | | | | RHD*weakDtype4.1/RHD*DAR-E | Weak or Partial D, C⁺ |
| | | | | | RHD*weakDtype14/RHD*DAR-E | Weak or Partial D, C⁺ |
| | | | | | RHD*weakDtype51/RHD*DAR-E | Weak or Partial D, C⁺ |
| | | | | | RHD*DAR/RHD*DAR-E | Partial D, C⁺ |
| | | | | | RHD*DAR-E/RHD*DAR-E | Partial D, C⁺ |
| Present | Absent | Present | G | G/C | Not Reported | |
| Present | Absent | Present | G | C | Not Reported | |
| Present | Absent | Present | G | Absent | Not Reported | |
| Present | Absent | Present | Absent | G | Not Reported | |
| Present | Absent | Present | Absent | G/C | Not Reported | |
| Present | Absent | Present | Absent | C | Not Reported | |
| Present | Absent | Present | Absent | Absent | RHD*ex04-ex07del/RHD*ex04-ex07del | D⁻, C⁺ |
| | | | | | RHD*ex04-ex07del/No RHD | D⁻, C⁺ |
| Present | Absent | Absent | C | G | RHD*ex03del/RHD*ex03del | D⁻, C⁺ |
| | | | | | RHD*ex03del/No RHD | D⁻, C⁺ |
| Present | Absent | Absent | C | G/C | Not Reported | |
| Present | Absent | Absent | C | C | Not Reported | |
| Present | Absent | Absent | C | Absent | Not Reported | |
| Present | Absent | Absent | C/G | G | Not Reported | |
| Present | Absent | Absent | C/G | G/C | Not Reported | |
| Present | Absent | Absent | C/G | C | Not Reported | |
| Present | Absent | Absent | C/G | Absent | Not Reported | |
| Present | Absent | Absent | G | G | Not Reported | |
| Present | Absent | Absent | G | G/C | Not Reported | |
| Present | Absent | Absent | G | C | Not Reported | |
| Present | Absent | Absent | G | Absent | Not Reported | |
| Present | Absent | Absent | Absent | G | RHD*ex03-ex04del/RHD*ex03-ex04del | D⁻, C⁺ |
| | | | | | RHD*ex03-ex04del/No RHD | D⁻, C⁺ |
| Present | Absent | Absent | Absent | G/C | Not Reported | |
| Present | Absent | Absent | Absent | C | Not Reported | |
| Present | Absent | Absent | Absent | Absent | No RHD/No RHD | D⁻, C⁺ |

TABLE 2

| Antigen | Phenotype 1 | Phenotype 2 | Sample Phenotype |
|---|---|---|---|
| RhD | D⁻ | D⁻ | D⁻ |
| | D⁻ | Weak D | Weak D |
| | D⁻ | Partial D | Partial D |
| | D⁻ | D⁺ | D⁺ |
| | Weak D | Weak D | Weak D |
| | Weak D | Partial D | Weak or Partial D |
| | Weak D | D⁺ | D⁺ |
| | Partial D | Partial D | Partial D |
| | Partial D | D⁺ | D⁺ |
| | D⁺ | D⁺ | D⁺ |
| RhC | C⁻ | C⁻ | C⁻ |
| | C⁻ | C^{+W} | C^{+W} |
| | C⁻ | C⁺ | C⁺ |
| | C^{+W} | C^{+W} | C^{+W} |
| | C^{+W} | C⁺ | C⁺ |
| | C⁺ | C⁺ | C⁺ |

### EXAMPLES

### Identification of Genetic Variants that Encode no D Antigen (D) and Altered C Antigen (C^{+W})

The following example relates to a method of identifying RHD*DIIIa-CE(4-7)-D or RHD*DIIIa-CE(4-7)-D)-like variants. The method described herein has been applied to 58 samples previously known to contain RHD/RHCE hybrid exon 3. The process described below proceeds from the genotyping of said samples and the subsequent analysis of said samples grouped by genotype and/or predicted phenotype. The serotype assigned to a group corresponds to analysis performed only on a subset of the samples in said group.

### Materials & Methods

Genomic DNA was extracted from nucleated cells in a blood sample by cell lysis. Extracted DNA was purified on an affinity column. Both cell lysis and DNA purification were performed with a QIAamp Blood kit (Qiagen, Germany) by following manufacturer protocols and recommendations. Purity of DNA was determined by spectrophotometry on a Nanodrop instrument (Nanodrop, DE). Only DNA solutions with an OD_{260/280} 1.8±0.2 proceeded to subsequent analysis.

Purified DNA was used as a template for multiplexed Polymerase Chain Reaction (PCR) amplification of the gene segments of interest in a GeneAmp 9700 thermal cycler (Perkin-Elmer, CA). Primer sequences for the different segments are listed in the Technical Description section below. Cycling conditions consisted of a denaturation/polymerase activation step at 95° C. for 15 min, followed by 38 cycles of denaturation at 95° C. for 45 sec, annealing at 60° C. for 60 sec, extension at 72° C. for 90 sec, and a final extension step at 72° C. for 10 min.

Amplified DNA was enzymatically fragmented by incubation with DNase I (Promega, WI) and alkaline phosphatase (Roche, Germany) at 37° C. for 30 min, followed by enzyme inactivation at 95° C. for 10 min.

Fragmented DNA was labeled by incubation with TdT enzyme (Roche, Germany) and biotin-ddUTP (Perkin-Elmer, CA) or Cy5-dCTP (Perkin-Elmer, CA) at 37° C. for 60 min.

Labelled DNA was placed on a Progenika proprietary microarray. The microarray comprised a modified crystal surface to which allele-specific oligonucleotide probes were covalently attached. Probes were designed to interrogate multiple allelic variant positions (i.e. markers) in the amplified genomic segments. Each allelic variant was interrogated by 2 probes, for a total of 4 probes per marker. Each probe was printed 10 times on the microarray, for a total of 40 features (spots) per SNP. Probe sequences are listed in the Technical Description section below. The labelled DNA/microarray interface was placed in an incubation chamber of a HS 4800 Pro station (Tecan, Switzerland) and incubated at 47° C. for 30 min and at 45° C. for 60 min in buffer containing SSPE, dextran, and deionized formamide to allow for probes to hybridize (bind) to their cognate sequences, when present. Unbound DNA was washed off by incubation at 23° C. for various times with buffer containing SSC with or without SDS. A streptavidin-Cy3 conjugate (Invitrogen, CA) diluted in buffer containing PBS and Tween-20 was added to the microarray surface and further incubated at 37° C. for 10 min. Unbound conjugate was washed off as before. The microarray was dried by flushing high-pressure liquid nitrogen through the incubation chamber.

Hybridized DNA was fluorescently labelled, either directly with Cy5 (by the transferase reaction above) or via the interaction between streptavidin-Cy3 conjugate and biotin (last steps of the hybridization), and immobilized on the microarray by sequence specific base pairing to their respective probe. Microarray-bound Cy3 and Cy5 fluorescence was detected on an InnoScan 710 confocal scanner (Innopsys, France). This scanner uses two laser beams with appropriate wavelengths for excitation of the Cy3 and Cy5 fluorophores and PMT sensors for the detection of the fluorescence signals generated. The fluorescence signal of each feature was subsequently quantified by ad hoc software.

Progenika proprietary software was used to transform fluorescence intensity values for the particular allelic variants detected, singly or in combination, into blood group genotypes, and from genotypes into predicted blood group phenotypes.

Serology analysis may be performed using methods well known to the skilled person. Suitable protocols may be found in, for example, The Blood Group Antigen FactsBook, Second edition. 2004. M. E. Reid and C. Lomas-Francis, Elsevier Ltd., and references cited therein to serology technical manuals.

### Technical Description

According to the present example, amplifications and hybridizations for determination of the five genetic sequences were performed as follows:

### Amplification of RHCE*C Intron 2 by PCR.

The following primers were used, which yielded a PCR product with a size of 357 base

| (SEQ ID NO: 3) | |
|---|---|
| Forward primer: | 5′-GGCCACCACCATTTGAA-3′ |
| (SEQ ID NO: 4) | |
| Reverse primer: | 5′-CCATGAACATGCCACTTCAC-3′ |

In boldface, RHCE*C-specific nucleotides
(forward primer).

### Amplification of RHD/RHCE Hybrid Exon 3 by PCR.

The following primers were used, which yielded a PCR product with a size of 256 base pairs:

(SEQ ID NO: 9)
Forward primer: 5′-TCCTGGCTCTCCCTCTCT-3′
(SEQ ID NO: 10)
Reverse primer: 5′-TTTTCAAAACCCCGGAAG-3′
In boldface, RHD-specific nucleotides (forward
primer) and RHCE-specific nucleotides (reverse
primer).

### Amplification of RHD Exon 3 by PCR.

The following primers were used, which yielded a PCR product with a size of 268 base pairs:

(SEQ ID NO: 15)
Forward primer: 5′-TCCTGGCTCTCCCTCTCT-3′
(SEQ ID NO: 16)
Reverse primer: 5′-GTTGTCTTTATTTTTCAAAACCCT-3′
In boldface, RHD-specific nucleotides.

### Amplification of RHD Exon 4 by PCR.

The following primers were used, which yielded a PCR product with a size of 281 base pairs:

(SEQ ID NO: 17)
Forward primer: 5′-GCTCTGAACTTTCTCCAAGGACT-3′
(SEQ ID NO: 18)
Reverse primer: 5′-ATTCTGCTCAGCCCAAGTAG-3′
In boldface, RHD-specific nucleotides.

### Amplification of RHD Exon 7 by PCR.

The following primers were used, which yielded a PCR product with a size of 695 base pairs:

(SEQ ID NO: 35)
Forward primer: 5′-ACAAACTCCCCGATGATGTGAGTG-3′
(SEQ ID NO: 36)
Reverse primer: 5′-GAGGCTGAGAAAGGTTAAGCCA-3′
In boldface, RHD-specific nucleotides.

### Hybridization of RHCE*C Intron 2 Amplicon to Oligonucleotide Probes.

The following probe sequences were used to determine the presence or absence of this amplicon:

(SEQ ID NO: 5)
RHCE*C-specific perfect match probe #1:
5′-TTTTACAGACGCCTGCTACCATG-3′
(SEQ ID NO: 6)
RHCE*C-specific perfect match probe #2:
5′-CATGGTAGCAGGCGTCTGTAAAA-3′
(SEQ ID NO: 7)
Mismatch probe #1: 5′-TTTTACAGACGTCTGCTACCATG-3′
(SEQ ID NO: 8)
Mismatch probe #2: 5′-CATGGTAGCAGACGTCTGTAAAA-3′
In boldface, the central position mismatch.

### Hybridization of RHD Exon 3 Amplicon or RHD/RHCE Hybrid Exon 3 Amplicon to Oligonucleotide Probes.

The following probe sequences were used to determine the presence or absence of both amplicons, using the SNP located at position 410 of both RHD and RHD/RHCE exon 3. The two amplicons were distinguished by using different label molecules (Cy5-dCTP or biotin-ddUTP) in the terminal transferase reaction described above.

RHD, RHCE wild-type probe #1:

| | |
|---|---|
| 5′-GGTCAACTTGGCGCAGTTGGTGG-3′ | (SEQ ID NO: 11) |

RHD, RHCE wild-type probe #2:

| | |
|---|---|
| 5′-GTCAACTTGGCGCAGTTGGTG-3′ | (SEQ ID NO: 12) |

Hybrid exon 3 variant probe #1:

| | |
|---|---|
| 5′-GGTCAACTTGGTGCAGTTGGTGG-3′ | (SEQ ID NO: 13) |

Hybrid exon 3 variant probe #2:

| | |
|---|---|
| 5′-GTCAACTTGGTGCAGTTGGTG-3′ | (SEQ ID NO: 14) |

In boldface, the SNP.

### Hybridization of RHD Exon 4 Amplicon to Oligonucleotide Probes

The following probe sequences were used to determine the presence, absence and SNP variant (either C or G) for the SNP located at position 602 of the coding sequence:

RHD wild-type probe #1:
(SEQ ID NO: 23)
5′-ATAAAGATCAGACAGCAACGATACC-3′
RHD wild-type probe #2:
(SEQ ID NO: 24)
5′-TAAAGATCAGACAGCAACGATAC-3′
RHD*DIIIα variant probe #1:
(SEQ ID NO: 25)
5′-ATAAAGATCAGAGAGCAACGATACC-3′
RHD*DIIIα variant probe #2:
(SEQ ID NO: 26)
5′-TAAAGATCAGAGAGCAACGATAC-3′
In boldface, the SNP.

### Hybridization of RHD Exon 7 Amplicon to Oligonucleotide Probes.

The following probe sequences were used to determine the presence, absence and SNP variant (either G or C) for the SNP located at position 1048 of the coding sequence:

| | |
|---|---|
| RHD wild-type probe #1: | |
| 5′-TGCTGGTGCTTGATACCGTCGGA-3′ | (SEQ ID NO: 37) |
| RHD wild-type probe #2: | |
| 5′-GCTGGTGCTTGATACCGTCGG-3′ | (SEQ ID NO: 38) |
| RHD*DIVα-2 variant probe #1: | |
| 5′-TGCTGGTGCTTCATACCGTCGGA-3′ | (SEQ ID NO: 39) |
| RHD*DIVα-2 variant probe #2: | |
| 5′-GCTGGTGCTTCATACCGTCGG-3′ | (SEQ ID NO: 40) |
| In boldface, the SNP. | |

### Grouping of Samples by Genotype Combination

Analysis by the method described above of 146 samples previously known to contain RHD/RHCE hybrid exon 3 yielded the results shown below. Samples were grouped by genotype and/or predicted phenotype. Serotype analysis was also performed on a subset of the samples in each group. Serotype analysis is unable to distinguish between C⁺ and C^{+W} antigen phenotypes, demonstrating the inaccurate results that can be obtained using this method. There are also some types of partial, weak and D− phenotypes that cannot be distinguished by serology.

### Group 1

### Number of Samples: 11

### Sample IDs: 09-0084, 10-0210, 10-0380, 10-0635, 10-0972, 10-2366, 10-2367, 10-3113, 10-3649, 10-3664, 10-3809

Genotyping data: RHCE*C intron 2 present, RHD/CE Hex03 present, RHD exon 3 present, RHD exon 4 602C, RHD exon 7 1048G.
RHD haplotype 1: RHD*DIIIa-CE(4-7)-D
   RHD haplotype 2: Possibly RHD*D
   Predicted RHD phenotype: D?
   Serotype: Not available
RHCE haplotype 1: RHCE*c
   RHCE haplotype 2: RHCE*C
   Predicted RHCE C phenotype: C⁺
   Serotype: C⁺

Determining the markers described herein correctly predicted that the clinical phenotype was not RHD*DIIIa-CE(4-7)-D, RHD*DIIIa or RHD*DIVa-2.

### Group 2

### Number of Samples: 49

Sample IDs: 09-0216, 09-0294, 10-0056, 10-0097, 10-0118, 10-0280, 10-0367, 10-0371, 10-0373, 10-0376, 10-0389, 10-0396, 10-0428, 10-0461, 10-0476, 10-0575, 10-0598, 10-0654, 10-0752, 10-0773, 10-0790, 10-0849, 10-0867, 10-0933, 10-1391, 10-1423, 10-1500, 10-1591, 10-1599, 10-1643, 10-1653, 10-2038, 10-2153, 10-2155, 10-2212, 10-2321, 10-2347, 10-2758, 10-3387, 10-3400, 10-3417, 10-3426, 10-3486, 10-3528, 10-3545, 10-3625, 10-3635, 10-3684, 10-3694
Genotyping data: RHCE*C intron 2 absent, RHD/CE Hex03 present, RHD exon 3 present, RHD exon 4 602C, RHD exon 7 1048G.
RHD haplotype 1: RHD*DIIIa-CE(4-7)-D
   RHD haplotype 2: Possibly RHD*D
   Predicted RHD phenotype: D?
   Serotype: D⁺
RHCE haplotype 1: RHCE*c
   RHCE haplotype 2: RHCE*c
   Predicted RHCE C phenotype: C^{+W}
   Serotype: C⁺

The method determining the markers described herein predicted that the clinical phenotype could be RHD*DIIIa-CE(4-7)-D, but could not be RHD*DIIIa or RHD*DIVa-2. For the patients actually tested, the D antigen was present, and therefore the phenotype was not RHD*DIIIa-CE(4-7)-D. These data also show the serotype analysis incorrectly reported a C⁺ phenotype instead of C^{+W}; in the absence of RHCE*C intron 2, the actual phenotype could not be C⁺.

### Group 3

### Number of Samples: 24

### Sample IDs: 10-0085, 10-0177, 10-0443, 10-0656, 10-0715, 10-0847, 10-0853, 10-0900, 10-1374, 10-1455, 10-1532, 10-1577, 10-1588, 10-1649, 10-1661, 10-2220, 10-2238, 10-2335, 10-3392, 10-3427, 10-3461, 10-3561, 10-3718, 10-4060

Genotyping data: RHCE*C intron 2 absent, RHD/CE Hex03 present, RHD exon 3 absent, RHD exon 4 602 absent, RHD exon 7 1048 absent.
RHD haplotype 1: RHD*DIIIa-CE(4-7)-D
   RHD haplotype 2: RHD*DIIIa-CE(4-7)-D or RHD*Ø
   Predicted RHD phenotype: D⁻
   Serotype: D⁻
RHCE haplotype 1: RHCE*c
   RHCE haplotype 2: RHCE*c
   Predicted RHCE C phenotype: C^{+W}
   Serotype: C⁺

The method described herein predicted a RHD*DIIIa-CE(4-7)-D phenotype; however, the serotype analysis incorrectly reported a C⁺ antigen phenotype, due to the inability to distinguish C⁺ from C^{+W}.

### Group 4

### Number of Samples: 32

Sample IDs: 09-0275, 09-0300, 10-0041, 10-0074, 10-0107, 10-0425, 10-0481, 10-0523, 10-0579, 10-0590, 10-0628, 10-0642, 10-0669, 10-0717, 10-0770, 10-0842, 10-0942, 10-1233, 10-1413, 10-1458, 10-1468, 10-1574, 10-1658, 10-1683, 10-2215, 10-2391, 10-2433, 10-2435, 10-2456, 10-3546, 10-3574, 10-4080
Genotyping data: RHCE*C intron 2 absent, RHD/CE Hex03 present, RHD exon 3 present, RHD exon 4 602C/G (heterozygous), RHD exon 7 1048G.
RHD haplotype 1: RHD*DIIIa
   RHD haplotype 2: Possibly RHD*D
   Predicted RHD phenotype: D?
   Serotype: D⁺
RHCE haplotype 1: RHCE*c
   RHCE haplotype 2: RHCE*c
   Predicted RHCE C phenotype: C⁻
   Serotype: C⁻

The method predicted that the phenotype was not due to a RHD*DIIIa-CE(4-7)-D haplotype. Serotype data agreed with these results.

### Group 5

### Number of Samples: 8

Sample IDs: 10-0420, 10-0512, 10-0543, 10-0735, 10-1634, 10-2379, 10-2470, 10-3409 Genotyping data: RHCE*C intron 2 present, RHD/CE Hex03 present, RHD exon 3 present, RHD exon 4 602C/G (heterozygous), RHD exon 7 1048G.
RHD haplotype 1: RHD*DIIIa
   RHD haplotype 2: Possibly RHD*D
   Predicted RHD phenotype: D?
   Serotype: D⁺
RHCE haplotype 1: RHCE*c
   RHCE haplotype 2: RHCE*C
   Predicted RHCE C phenotype: C⁺
   Serotype: C⁺

The method correctly predicted that the clinical phenotype was not RHD*DIIIa-CE(4-7)-D, RHD*DIIIa or RHD*DIVa-2.

### Group 6

### Number of Samples: 10

### Sample ID: 09-0032, 10-0187, 10-0281, 10-1379, 10-1621, 10-1628, 10-2142, 10-2506, 10-3051, 10-3097

Genotyping data: RHCE*C intron 2 absent, RHD/CE Hex03 present, RHD exon 3 absent, RHD exon 4 602G, RHD exon 7 1048G.
RHD haplotype 1: RHD*DIIIa
   RHD haplotype 2: RHD*DIIIa or RHD*DIIIa-CE(4-7)-D or RHD*Ø⁽¹⁾
   Predicted RHD phenotype: Partial D
RHCE haplotype 1: RHCE*c
   RHCE haplotype 2: RHCE*c
   Predicted RHCE C phenotype: C⁻ or C^{+W}

No serotype data available for these samples.

### Group 7

### Number of Samples: 4

### Sample IDs: 09-0287, 09-0333, 10-0075, 10-0284

Genotyping data: RHCE*C intron 2 absent, RHD/CE Hex03 present, RHD exon 3 present, RHD exon 4 602G, RHD exon 71048G.
RHD haplotype 1: RHD*DIIIa or RHD*DIIIa-CE(4-7)-D
   RHD haplotype 2: RHD*weakD
   Predicted RHD phenotype: Weak D or Partial D
   Serotype: D⁺
RHCE haplotype 1: RHCE*c
   RHCE haplotype 2: RHCE*c
   Predicted RHCE C phenotype: C⁻ or C^{+W}
   Serotype: Data not available

The method correctly predicted that the clinical phenotype was not RHD*DIIIa-CE(4-7)-D.

### Group 8

### Number of Samples: 5

### Sample IDs: 10-0052, 10-0638, 10-0723, 10-0876, 10-2144

Genotyping data: RHCE*C intron 2 absent, RHD/CE Hex03 present, RHD exon 3 present, RHD exon 4 602C, RHD exon 7 1048G/C (heterozygous).
RHD haplotype 1: RHD*DIVa-2
   RHD haplotype 2: Possibly RHD*D
   Predicted RHD phenotype: D?
RHCE haplotype 1: RHCE*c
   RHCE haplotype 2: RHCE*c
   Predicted RHCE C phenotype: C⁻

No serotype data available for these samples.

### Group 9

### Number of Samples: 3

### Sample IDs: 10-0081, 10-0387, 10-0400

Genotyping data: RHCE*C intron 2 present, RHD/CE Hex03 present, RHD exon 3 present,
RHD exon 4 602C, RHD exon 7 1048G/C.
   RHD haplotype 1: RHD*DIVa-2
   RHD haplotype 2: Possibly RHD*D
   Predicted RHD phenotype: D?
   Serotype: D⁺
RHCE haplotype 1: RHCE*c
   RHCE haplotype 2: RHCE*C
   Predicted RHCE C phenotype: C⁺
   Serotype: C⁺

The method correctly predicted that the clinical phenotype was not RHD*DIIIa-CE(4-7)-D, RHD*DIIIa or RHD*DIVa-2.

⁽¹⁾RHD*Ø: No RHD gene.

The above results are summarized in Table 3.

TABLE 3

| | | No. Haplotypes | % Haplotypes |
|---|---|---|---|
| Hybrid Exon | RHD*DIIIa- | 84 | 28.8 |
| 3 Variant | CE(4-7)-D | | |
| | RHD*DIIIa | 50 | 17.1 |
| | RHD*DIVa-2 | 8 | 2.7 |
| | Uncertain | 38 | 13.0 |
| Other | | 112 | 38.4 |
| Total | | 292 | 100.0 |
| | | No. Samples | % Samples |
| RHD*DIIIa- | Present | 84 | 57.5 |
| CE(4-7)-D | Absent | 48 | 32.9 |
| Call | Uncertain | 14 | 9.6 |
| | Total | 146 | 100.0 |
| | | No. Samples | % Samples |
| Predicted RhD | D? | 108 | 74.0 |
| Phenotype | Partial D | 10 | 6.9 |
| | D⁻ | 24 | 16.4 |
| | Uncertain | 4 | 2.7 |
| | Total | 146 | 100.0 |
| | | No. Samples | % Samples |
| Predicted RhC | C⁺ | 22 | 15.1 |
| Phenotype | C^{+W} | 73 | 50.0 |
| | C⁻ | 37 | 25.3 |
| | Uncertain | 14 | 9.6 |
| | Total | 146 | 100.0 |

These data show that using the markers described herein, it is possible to distinguish RHD*DIIIa-CE(4-7)-D from RHD*DIIIa or RHD*DIVa-2. Further, these data show that relying on serotype analysis can erroneously lead to the incorrect diagnosis of a C^{+W} antigen phenotype as C.

### REFERENCES

1. DIIIa and DIII Type 5 are encoded by the same allele and are associated with altered RHCE*ce alleles: clinical implications. Connie M. Westhoff, Sunitha Vege, Christine Halter-Hipsky, Trina Whorley, Kim Hue-Roye, Christine Lomas-Francis, and Marion E. Reid. Transfusion (2010) 50, pp. 1303-1311.
2. Heterogeneous molecular background of the weak C, VS+, hrB−, HrB− phenotype in black persons. Bach-Nga Pham, Thierry Peyrard, Genevieve Juszczak, Isabelle Dubeaux, Dominique Gien, Antoine Blancher, Jean-Pierre Cartron, Philippe Rouger, and Pierre-Yves Le Pennec. Transfusion (2009) 49, pp. 495-504.
3. RHC and RHc genotyping in different ethnic groups. Martine G. H. M. Tax, C. Ellen van der Schoot, René van Doom, Lotte Douglas-Berger, Dick J. van Rhenen, and Petra A. Maaskant-van Wijk. Transfusion (2002) 42, pp. 6234-644.
4. The Blood group antigen FactsBook, Second edition. 2004. M. E. Reid and C. Lomas-Francis. Elsevier Ltd.

We claim:

1. A method of discriminating the RHD*DIIIa-CE(4-7)-D or RHD*DIIIa-CE(4-7)-D)-like blood type variants, which express the C^{+W} antigen and lack a D antigen, from RHD*DIIIa, RHD*DIVa-2 and other blood type variants, the method comprising:
determining at least 4 markers in a sample that has been obtained from the subject, wherein the markers comprise:
(i) the presence or absence of an RHCE*C allele;
(ii) the presence or absence of an RHD/RHCE hybrid exon 3 (RHD/CE Hex03) allele;
(iii) the absence of, or a single nucleotide polymorphism (SNP) variant within, any one of RHD exon 4, RHD exon 5, or RHD exon 6; and
(iv) the absence of, or SNP variant within, RHD exon 7.

2. The method according to claim 1, wherein:
a) the SNP variant within RHD exon 4 is at position 602 of the RHD coding sequence (rs1053355),
b) the SNP variant within RHD exon 5 is at position 667 of the RHD coding sequence (rs1053356),
c) the SNP variant within RHD exon 6 is at position 819 of the RHD coding sequence, as set forth in SEQ ID NO: 1; and/or
d) the SNP variant within RHD exon 7 is at position 1048 of the RHD coding sequence (rs41307826).

3. The method according to claim 1, wherein the markers further comprise:
(v) the presence or absence of an RHD exon 3 allele.

4. The method according to claim 1, wherein the method further comprises determining the RHD and RHC antigen phenotypes of the subject.

5. The method according to claim 1, wherein the method comprises detecting the presence or absence of a blood type variant selected from the group consisting of: RHD*DIIIa; RHD*DIVa-2; RHD*DIIIa-CE(4-7)-D; and RHD*DIIIa-CE(4-7)-D)-like blood type variants.

6. The method according to claim 1, wherein said marker (iii) is the SNP within RHD exon 4 at position 602 of the RHD coding sequence (rs1053355).

7. The method according to claim 1, wherein the RHCE*C allele is determined by determining:
(i) the presence or absence of RHCE*C intron 2; or
(ii) a nucleotide position in the RHCE coding sequence selected from the group of RHCE coding sequence nucleotide positions consisting of: position 307 in exon 2; position 48 in exon 1; position 150 in exon 2, position 178 in exon 2; position 201 in exon 2; and position 203 in exon 2.

8. The method according to claim 1, wherein the sample comprises nucleic acid and the method comprises amplifying the nucleic acid or a portion thereof by PCR using primers.

9. The method according to claim 8, wherein the PCR primers for determining the RHCE*C allele comprise:

| (SEQ ID NO: 3) | |
|---|---|
| Forward: | 5′-GGCCACCACCATTTGAA-3′ |
| (SEQ ID NO: 4) | |
| Reverse: | 5′-CCATGAACATGCCACTTCAC-3′, |

or a variant thereof having up to 4 nucleotide alterations.

10. The method according to claim 8, wherein the PCR primers for determining the RHD/CE Hex03 allele comprise:

| (SEQ ID NO: 9) | |
|---|---|
| Forward primer: | 5′-TCCTGGCTCTCCCTCTCT-3′ |
| (SEQ ID NO: 10) | |
| Reverse primer: | 5′-TTTTCAAAACCCCGGAAG-3 |

or a variant thereof having up to 4 nucleotide alterations.

11. The method according to claim 8, wherein the PCR primers for determining the SNP within RHD exon 4 at position 602 of the RHD coding sequence (rs1053355) comprise:
(SEQ ID NO: 17)

| | |
|---|---|
| Forward primer: | 5′-GCTCTGAACTTTCTCCAAGGACT-3′ |

(SEQ ID NO: 18)

| | |
|---|---|
| Reverse primer: | 5′-ATTCTGCTCAGCCCAAGTAG-3′ |

or a variant thereof having up to 4 nucleotide alterations.

12. The method according to claim 8, wherein the PCR primers for determining the SNP within RHD exon 5 at position 667 of the RHD coding sequence (rs1053356) comprise:

| (SEQ ID NO: 19) | |
|---|---|
| Forward primer: | 5′-TTGAATTAAGCACTTCACAGAGCA-3′ |
| (SEQ ID NO: 20) | |
| Reverse primer: | 5′-CACCTTGCTGATCTTCCC-3′ |

or a variant thereof having up to 4 nucleotide alterations.

13. The method according to claim 8, wherein the PCR primers for determining the SNP within RHD exon 6 at position 819 of the RHD coding sequence comprise:

| (SEQ ID NO: 21) | |
|---|---|
| Forward primer: | 5′-AGTAGTGAGCTGGCCCATCA-3′ |
| (SEQ ID NO: 22) | |
| Reverse primer: | 5′-CTTCAGCCAAAGCAGAGGAG-3′ |

or a variant thereof having up to 4 nucleotide alterations.

14. The method according to claim 8, wherein the PCR primers for determining the SNP within RHD exon 7 at position 1048 of the RHD coding sequence (rs41307826) comprise:

| (SEQ ID NO: 35) | |
|---|---|
| Forward primer: | 5′-ACAAACTCCCCGATGATGTGAGTG-3′ |
| (SEQ ID NO: 36) | |
| Reverse primer: | 5′-GAGGCTGAGAAAGGTTAAGCCA-3′ |

or a variant thereof having up to 4 nucleotide alterations.

15. The method according to claim 8, wherein said markers further comprise the presence or absence of an RHD exon 3 allele, and wherein the PCR primers for determining the RHD exon 3 allele comprise:

| (SEQ ID NO: 15) | |
|---|---|
| Forward primer: | 5′-TCCTGGCTCTCCCTCTCT-3′ |
| (SEQ ID NO: 16) | |
| Reverse primer: | 5′-GTTGTCTTTATTTTTCAAAACCCT-3′ |

or a variant thereof having up to 4 nucleotide alterations.

16. The method according to claim 8, wherein the amplified nucleic acid comprises a label.

17. The method according to claim 16, wherein the label comprises a biotinylated nucleotide.

18. The method according to claim 16, wherein the label comprises a fluorescent moiety.

19. The method according to claim 1, wherein the sample comprises nucleic acid, and the method comprises amplifying the nucleic acid or a portion thereof by PCR using primers, fragmenting the amplified nucleic acid, and labelling the fragmented nucleic acid with biotinylated ddNTPS using a terminal deoxynucleotidyl transferase (TdT) enzyme.

20. The method according to claim 1, wherein determining the presence, absence or SNP variant of a marker comprises contacting nucleic acid containing each marker with one or more probes.

21. The method according to claim 20, wherein the one or more probes comprise one or more probes selected from the group consisting of:

| | |
|---|---|
| 5′-TTTTACAGACGCCTGCTACCATG-3′, | (SEQ ID NO: 5) |
| 5′-CATGGTAGCAGGCGTCTGTAAAA-3′, | (SEQ ID NO: 6) |
| 5′-TTTTACAGACGTCTGCTACCATG-3′, | (SEQ ID NO: 7) |
| 5′-CATGGTAGCAGACGTCTGTAAAA-3′, | (SEQ ID NO: 8) |
| 5′-ATAAAGATCAGACAGCAACGATACC-3′ | (SEQ ID NO: 23) |
| 5′-TAAAGATCAGACAGCAACGATAC-3′ | (SEQ ID NO: 24) |
| 5′-ATAAAGATCAGAGAGCAACGATACC-3′ | (SEQ ID NO: 25) |
| 5′-TAAAGATCAGAGAGCAACGATAC-3′ | (SEQ ID NO: 26) |
| 5′-CTGGCCAAGTTTCAACTCTGC-3′ | (SEQ ID NO: 27) |
| 5′-TGGCCAAGTTTCAACTCTG-3′ | (SEQ ID NO: 28) |
| 5′-CTGGCCAAGTGTCAACTCTGC-3′ | (SEQ ID NO: 29) |
| 5′-TGGCCAAGTGTCAACTCTG-3′ | (SEQ ID NO: 30) |
| 5′-GTGCACAGTGCGGTGTTGGCAGG-3′ | (SEQ ID NO: 31) |
| 5′-TGCACAGTGCGGTGTTGGCAG-3′ | (SEQ ID NO: 32) |
| 5′-GTGCACAGTGCAGTGTTGGCAGG-3′ | (SEQ ID NO: 33) |
| 5′-TGCACAGTGCAGTGTTGGCAG-3′ | (SEQ ID NO: 34) |
| 5′-TGCTGGTGCTTGATACCGTCGGA-3′ | (SEQ ID NO: 37) |
| 5′-GCTGGTGCTTGATACCGTCGG-3′ | (SEQ ID NO: 38) |
| 5′-TGCTGGTGCTTCATACCGTCGGA-3′; | (SEQ ID NO: 39) |
| and | |
| 5′-GCTGGTGCTTCATACCGTCGG-3′, | (SEQ ID NO: 40) |

or a variant of any one of said probes 1 to 4 having up to 4 nucleotide alterations.

22. The method according to claim 20, wherein one or more of the probes comprise a label.

23. The method according to claim 20, wherein one or more of the probes is attached to a solid support or conjugated to one or more particles.

24. A set of primers for amplifying nucleic acid comprising at least two primers selected from the group consisting of:

| | |
|---|---|
| Forward: | |
| 5′-GGCCACCACCATTTGAA-3′; | (SEQ ID NO: 3) |
| Reverse: | |
| 5′-CCATGAACATGCCACTTCAC-3′,; | (SEQ ID NO: 4) |
| Forward: | |
| 5′-TCCTGGCTCTCCCTCTCT-3′; | (SEQ ID NO: 9) |
| Reverse: | |
| 5′-TTTTCAAAACCCCGGAAG-3; | (SEQ ID NO: 10) |
| Forward: | |
| 5′-GCTCTGAACTTTCTCCAAGGACT-3′; | (SEQ ID NO: 17) |
| Reverse: | |
| 5′-ATTCTGCTCAGCCCAAGTAG-3′; | (SEQ ID NO: 18) |
| Forward: | |
| 5′-TTGAATTAAGCACTTCACAGAGCA-3′; | (SEQ ID NO: 19) |
| Reverse: | |
| 5′-CACCTTGCTGATCTTCCC-3′; | (SEQ ID NO: 20) |
| Forward: | |
| 5′-AGTAGTGAGCTGGCCCATCA-3′; | (SEQ ID NO: 21) |
| Reverse: | |
| 5′-CTTCAGCCAAAGCAGAGGAG-3′; | (SEQ ID NO: 22) |
| Forward: | |
| 5′-ACAAACTCCCCGATGATGTGAGTG-3′; | (SEQ ID NO: 35) |
| Reverse: | |
| 5′-GAGGCTGAGAAAGGTTAAGCCA-3′; | (SEQ ID NO: 36) |
| Forward: | |
| 5′-TCCTGGCTCTCCCTCTCT-3′; | (SEQ ID NO: 15) |
| and | |
| Reverse: | |
| 5′-GTTGTCTTTATTTTTCAAAACCCT-3′. | (SEQ ID NO: 16) |

25. A set of probes for determining the presence, absence or single nucleotide polymorphism (SNP) the set of probes comprising at least three probes selected from the group consisting of:

| | |
|---|---|
| 5′-TTTTACAGACGCCTGCTACCATG-3′, | (SEQ ID NO: 5) |
| 5′-CATGGTAGCAGGCGTCTGTAAAA-3′, | (SEQ ID NO: 6) |
| 5′-TTTTACAGACGTCTGCTACCATG-3′, | (SEQ ID NO: 7) |
| 5′-CATGGTAGCAGACGTCTGTAAAA-3′, | (SEQ ID NO: 8) |
| 5′-ATAAAGATCAGACAGCAACGATACC-3′ | (SEQ ID NO: 23) |
| 5′-TAAAGATCAGACAGCAACGATAC-3′ | (SEQ ID NO: 24) |
| 5′-ATAAAGATCAGAGAGCAACGATACC-3′ | (SEQ ID NO: 25) |
| 5′-TAAAGATCAGAGAGCAACGATAC-3′ | (SEQ ID NO: 26) |
| 5′-CTGGCCAAGTTTCAACTCTGC-3′ | (SEQ ID NO: 27) |
| 5′-TGGCCAAGTTTCAACTCTG-3′ | (SEQ ID NO: 28) |
| 5′-CTGGCCAAGTGTCAACTCTGC-3′ | (SEQ ID NO: 29) |
| 5′-TGGCCAAGTGTCAACTCTG-3′ | (SEQ ID NO: 30) |
| 5′-GTGCACAGTGCGGTGTTGGCAGG-3′ | (SEQ ID NO: 31) |
| 5′-TGCACAGTGCGGTGTTGGCAG-3′ | (SEQ ID NO: 32) |
| 5′-GTGCACAGTGCAGTGTTGGCAGG-3′ | (SEQ ID NO: 33) |
| 5′-TGCACAGTGCAGTGTTGGCAG-3′ | (SEQ ID NO: 34) |
| 5′-TGCTGGTGCTTGATACCGTCGGA-3′ | (SEQ ID NO: 37) |
| 5′-GCTGGTGCTTGATACCGTCGG-3′ | (SEQ ID NO: 38) |
| 5′-TGCTGGTGCTTCATACCGTCGGA-3′; | (SEQ ID NO: 39) |
| and | |
| 5′-GCTGGTGCTTCATACCGTCGG-3′. | (SEQ ID NO: 40) |

26. The set of probes according to claim 25, wherein the probes are immobilized on a solid support or conjugated to one or more particles.

27. The set of probes according to claim 25, wherein one or more of the probes comprises a label.

28. A kit for genotyping a subject, the kit comprising a set of PCR primers comprising at least two primers selected from the group consisting of SEQ ID NOs: 3, 4, 9, 10, 15-22, 35 and 36, and a set of probes comprising at least three probes selected from the group consisting of SEQ ID NOs: 5-8, 23-34 and 37-40.
